(19) United States
(12) Patent Application Publication
(10) Pub. No.: US 2009/0208951 A1
(43) Pub. Date: Aug. 20, 2009

(54) DETECTION OF T-DNA

(75) Inventor: Perikles Simon, Tubingen (DE)

Correspondence Address: KLARQUIST SPARKMAN, LLP, 121 SW SALMON STREET, SUITE 1600, PORTLAND, OR, 97204, US

(73) Assignee: Eberhard-Karls-Universitat Tubingen Universitatsklinikum

(21) Appl. No.: 12/259,810

(22) Filed: Oct. 28, 2008

Related U.S. Application Data
(63) Continuation of application No. PCT/EP2007/003385, filed on Apr. 18, 2007.

(30) Foreign Application Priority Data
Apr. 28, 2006 (DE) 10 2006 021 257.6

(51) Int. Cl. C12Q 1/68

(52) U.S. Cl. 435/6

(57) ABSTRACT

The present invention relates to a method for the detection of transgenic DNA (tDNA) in a living being and to a kit for performing such a method.

### CROSS REFERENCE TO RELATED APPLICATIONS

This application is a continuation of co-pending International Patent Application PCT/EP2007/003385 filed on Apr. 18, 2007 and designating the United States, which was published under PCT Article 21(2) in English, and claims priority of German Patent Application DE 10 2006 021 257 filed on Apr. 28, 2006, which is incorporated herein by reference.

### FIELD OF THE INVENTION

The present invention relates to a method for the detection of transgenic DNA (tDNA) in a living being and to a kit for performing such a method.

### BACKGROUND

So far a genetic manipulation of organisms can especially be detected if it occurs in terms of an alteration of the genome of the germ line, for example by a genetic manipulation of embryonic stem cells (ESC) or of such progenitor cells of a whole organism, which belong to the germ line. The consequence of the manipulation of germ line cells is that, depending on the used technology, the genetic modification is more or less reflected in each progeny cell of the progenitor cell and consequently in each cell of the growing-up and adult living being.

The conventional field of application for a manipulation of the germ line relates to the generation of so-called transgenic living beings by a genetic manipulation of ESC by means of gene transfer. The gene transmitted to the ESC is also referred to as a transgene and the transferred DNA as a transgenic DNA (tDNA). The tDNA typically originates from an organism different from the target organism and is, therefore, to be referred to as non-species homologous tDNA. In the conventional terminology of gene therapy tDNA refers to a DNA which might also be species homologous and which is introduced from the outside into a target cell of an organism. The resulting living being is referred to as a transgenic living being. An example relates to the creation of a transgenic “giant mouse” into which the tDNA of the growth hormone of the rat has been integrated; cf. Brinster and Palmiter (1986), Introduction of Genes into the Germ Lines of Animals, in Harvey Lectures 80, pages 1-38. This mouse attained twice the size of a normal mouse. In such a case, the detection of a technically successful genetic manipulation is simple since it can be directly and unambiguously concluded from the phenotype of the transgenic mouse which is larger than each of its litter-mates.

The genetic manipulation of a living being can however also result in subtle or merely gradual alterations which do not allow an immediate distinction between the genetically non-manipulated wild-type on the basis of the phenotype. Therefore, several detection methods have been developed by which it can be identified whether a successful gene transfer has been performed or not. Usually such a detection is carried out by means of biological material taken from the manipulated animal; cf. Schneider and Wolf (2005), Genotyping of transgenic mice: Old principles and recent developments, Analytical Biochemistry 344, pages 1-7, as the most current review about possible methods for the genotyping of transgenic animals. By means of PCR it can be succeeded in the detection of a successful manipulation of the germ line, performed on saliva, excrements or hairs, wherein only few cells or its fragments could be sufficient for a successful detection.

However, the prior art regarding the detection of a genetic manipulation of the germ line of a living being is fundamentally different to the prior art regarding the detection of a genetic manipulation of so-called somatic cells which do not belong to the germ line and consequently do not have the endogenous capacity to develop into a complete living being. Genetic manipulations of somatic cells are referred to as somatic gene therapy or gene doping. It is referred to a somatic gene therapy if tDNA is introduced into a living being with the object of the curing of a disease in a living being. It is referred to gene doping if in principle a method based on the same technology is used with the object of a performance enhancement in the living being.

So far, in relation to the somatic gene therapy the need for a detection method has not become an issue. This is based on the fact that 1. the patient and therapist are precisely informed about the performed genetic intervention and its technical modalities and under certain legal conditions third parties have to be informed about such intervention, 2. not the genetic manipulation is the object but the curing of the underlying disease, and as a result 3. so far not the detection of a genetic manipulation as such has been focused but merely the detection of a functionally effective modification. These circumstances may however change if the number of diseases will increase towards more harmless and asymptomatic diseases, which should be cured by somatic gene therapy. Here, different safety regulations for the treatment to be performed will apply, which require a detection of tDNA in a highly sensitive manner in each body's excrement, juice and each body's tissue.

The situation is different with gene doping. Here, the same technology and to a large degree also the same candidate genes like in the somatic gene therapy are used, whereas already these days there is a high interest in a method that enables the detection of an occurred genetic manipulation. It has to be emphasized that such a manipulation takes place by a species homologous tDNA like in the somatic gene therapy, which is hard to distinguish from the corresponding genomic DNA (gDNA) which is present in each body cell.

In the following, the technical problem of the detection of gene doping or also of somatic gene therapy will be set out in more detail and the prior art relating to such detection will be explained.

In sports, doping refers to the use of performance-enhancing methods. Usually such methods comprise the intake of certain substances by the athlete, which are derived from endogenic substances such as testosterone or growth factors, and which e.g. promote an increased muscle growth or the maturation of red blood cells. At international competitions the use of such performance-enhancing substances is strictly forbidden.

Several methods are used with the objective to ensure doping-free competitions, by which an athlete should be tested for the intake of such substances. It is preferred to detect the performance-enhancing substance directly in a biological sample originating from the athlete. This is, for example, possible if the substance differs from the endogenic substance due to its chemical structure. In this way, it can be succeeded in the detection of testosterone derivatives which have been modified over natural testosterone. In an equal manner it is frequently succeeded in the detection of exogenously administrated peptide hormones such as erythropoietin (EPO) since the latter differs in relation of its glycosylation pattern from endogenous EPO. The detection of the before-mentioned performance-enhancing substances within the scope of standard doping tests is primarily possible in principle since such substances can be found in the urine or blood of the doped athlete and such a sample can be taken from the athlete without any serious intervention in the physical integrity.

In the recent years a new kind of doping came into the spotlight, namely the so-called gene-doping. Gene-doping refers to a targeted transfer of selected genes or gene fragments into specific tissues or cells by means of several methods of the somatic gene therapy. These methods can be of biological nature, wherein the gene or gene fragment is introduced into the target tissue, for example into the musculature, via a viral or non-viral vector. Further methods are of physical nature and comprise the direct injection of the gene or gene fragment into the tissue or the cell by means of an ultrathin cannula or a so-called “gene cannon”. Methods of biochemical nature comprise the use of phospholipid vesicles or liposomes which contain the gene or gene fragments and are introduced into the organism. The introduction of the gene can occur directly in the body (in vivo), or it can be performed in a cell which was previously taken from the body, which after the genetic modification has taken place will be returned to the body (ex vivo), or a modification of non-endogenous cells is performed in a test tube, which after the modification are then re-introduced into the body.

It is expected that the most effective method for gene doping is realized by the use of genetically modified viral vectors which are derived from retroviruses, adenoviruses or lentiviruses, which are deficient in replication and contain the so-called “transgene”, i.e. the coding sequence of the gene product of interest. The genetically altered viruses are then introduced into the body where they infect cells and recruit the biochemical machinery of the cell in order to express the introduced transgene. With a suitable design of these vectors a long-lasting expression, a low anti-vector immunity, a cell-specific tropism and a high packaging capacity can be achieved.

An overview of the current state of the art in the field of gene doping can for example be found in H. Lee Sweeney (2004), Gene Doping, Scientific American, pages 37 to 43, and in Azzazy et al. (2005), Doping in the recombinant era: Strategies and counterstrategies, Clinical Biochemistry 38, pages 959-965.

H. Lee Sweeney and colleagues succeeded in the creation of a so-called “super mouse” by means of gene doping. For this, the gene for the insulin-like growth factor (IGF1) was directly introduced into the muscle via an adeno-associated virus (AAV). These mice presented a muscle mass that was increased about 30-40%, had a longer life span and recovered faster from injuries in comparison with the control mice. According to the information of the experimentalists, the transgene IGF1 which was expressed in the musculature and was only found in the muscle but not in blood or in urine. Additionally, the transgene IGF1 is identical with the endogenous IGF1 variant.

A method was recently published in a scientific journal which allegedly detects in the serum EPO which has been introduced into the body by means of gene doping; however such method has turned out as not being practicable. Lasne et al. (2004), Genetic Doping with erythropoietin cDNA in primate muscle is detectable, Molecular Therapy 10, Nr. 3, pages 109 und 110, assert that in Macaques which were injected with cDNA encoding EPO via a recombinant AAV into the skeletal muscles, such an EPO variant can be detected in blood, which comprises a different isoelectric pattern from physiological EPO. However, these results base on the use of a standard method for the detection of recombinant erythropoietin in urine. It was just recently shown that this method is useless since physical exercise can result in false-positive findings; cf. Beullens et al. (2006), False-positive detection of recombinant human erythropoietin in urine following strenuous physical exercise, Blood First Edition Paper, online-prepublication. The antibody for the detection of erythropoietin that was used by Lasne et al. (cit. loc.) has turned out as being non-monospecific and may therefore, under the burden of a gene therapy, lead to a false-positive result due to cross-reaction with an unknown stress-induced peptide.

Consequently, at present the experts are of the opinion that an athlete can only be found guilty of being gene doped in an indirect way on account of physiological alterations in the body which result from the expression of the transgene. Such an indirect detection of occurred gene doping has, however, the disadvantage that also such athletes would be identified as allegedly being doped which show an enhanced expression of doping-relevant proteins due to a natural genetic polymorphism. This would result in an accusation against non-doped athletes. Furthermore, athletes who have been found guilty in this manner in an appropriate good defense could refer to an alleged genetic favorism from birth, that such an indirect detection of gene doping would in many cases be unenforceable. Another problem with this approach is that the reactions of a body on heavy exercise in competitive sports but also reactions on ordinary diseases could be complex and extreme, so that an indirect detection of gene doping would always result in the question whether the observed alterations could not be explained by any other reason but a supposed gene doping.

The direct detection of gene doping is therefore according to the experts' opinion currently only possible by means of a well-directed biopsy of exactly that part of tissue which was genetically modified, for example the muscle. The muscle or the suspected tissue is then directly analyzed for the presence of the vector or the transgene. In the case of AAV it is frequently found that athletes have been infected with such harmless virus by a natural way that a conclusion to gene doping would be difficult. Furthermore, most of the athletes especially close to competition would not be willing to endure an invasive biopsy since for example muscle tissue will then be injured. In view of the somatic gene therapy it has to be considered that treated tissue may only comprise a transgene or vector at specific locations which might not be known to the controller. It has already been shown in an animal experiment that the transfer of a tDNA for the erythropoietin gene only into restricted parts of tissue of the body can result in a doping effect. Therefore, controllers do often not know which tissues are to be subjected to a biopsy.

Document WO 98/50580 describes a conventional PCR-based method for the detection of a neomycin-resistance gene, which has been introduced into the cells of a biological sample by means of a retroviral vector. This method, however, does not enable a differentiation between exogenously supplied and the homologous endogenous gene sequences.

Ayesh et al. (2006), A non-invasive QPCR method monitoring DNA based therapy of bladder cancer patients, Vaccine 24, pages 3420-3425, disclose a method for the detection of an expression vector which encodes the diphtheria toxin A in samples of blood and urine of a gene-therapeutically treated patient. However, this method also does not enable a differentiation between exogenously supplied and homologous endogenous gene sequences.

Schneider and Wolf (2005), Genotyping of transgenic mice: old principles and recent developments, Analytical Biochemistry 344, pages 1-7, describe a PCR-based method to detect tDNA in several biological samples. The authors propose to use such PCR primers for the PCR, which hybridize to several exons of the genes to be detected. The differentiation between the tDNA and the genomic DNA is then realized on account of the different sizes of the obtained amplificates. However, this method has turned out as being complex and unreliable.

### SUMMARY OF THE INVENTION

The foregoing and other objects, features, and advantages of the invention will become more apparent from the following detailed description, which proceeds with reference to the accompanying figures.

Against this background the object underlying the invention is to provide a reliable method for the detection of transgenic DNA (tDNA) in a living being, which is devoid of the disadvantages of the prior art. Especially such a method should be provided which enables a direct detection of the transgene by a justifiable invasive or also a non-invasive intervention in the living being, and by means of which false-positive results are largely excluded.

This object is achieved by the provision of a method for the detection of transgenic DNA (tDNA) in a living being, which comprises the following steps (1) provision of a biological sample originating from said living being, (2) analysis of said biological sample for the presence of tDNA, and (3) correlation of a positive finding in step (2) with a positive detection of tDNA in said living being, wherein said biological sample is a non-bioptic sample.

According to the invention transgenic DNA (tDNA) refers to such a nucleic acid molecule which encodes a transgene, where the transgene comprises the coding sequence for such a protein or a peptide which exhibits the wanted physiological, preferably performance-enhancing effect in an organism into which the tDNA has been introduced. According to the invention the tDNA relates to such a nucleic acid molecule which can be transferred into the living being to be analyzed in a targeted, preferably organ- or tissue-type specific and species homologous manner by means of the gene therapy. A transgene can be identical with a cDNA which derives from a natural gene or a so-called candidate gene, respectively, like in the case of erythropoietin (EPO), human growth hormone (hGH), insulin-like growth factor 1 (IGF1) etc. The tDNA can also comprise a coding sequence which differs from the cDNA of the underlying candidate gene, as this for example applies for myostatin. Whereas myostatin inhibits muscle growth in the organism, a tDNA derived from myostatin would be altered in its sequence in such a way that the inhibiting effect would be abolished.

Surprisingly, the inventor has found out that the tDNA can be detected in non-bioptic samples of the transfected living being. According to the invention, a non-bioptic sample refers to such a sample which comprises biological material and which can be obtained by avoidance of a biopsy from the living being by means of largely non-invasive methods from the living being. Non-bioptic samples encompass blood samples, saliva samples, urine samples, hair samples, excrement samples, as well as smear and liquid samples from the mouth, eyes, nose, rectal and genital area.

This finding was especially interesting since so far experts were of the opinion that tDNA can be exclusively detected in the transfected tissue or the transfected cell, respectively, however not in the before-mentioned samples, especially not in blood; cf. vgl. Sweeney (cit. loc.), page 43, left column, 3rd paragraph; S. Pincock (2005), Feature Gene doping, Lancet 366, page 18, right column, first paragraph; Azzazy et al. (cit. loc.) page 963, left column, last paragraph; L. DeFrancesco (2004), The faking of champions, Nature Biotechnology Vol. 22, Nr. 9, page 1070, right column, first paragraph.

The results from the somatic gene therapy do also point into this direction. Raper et al. (2003), Fatal Systemic inflammatory response syndrome in a ornithine transcarbamylase deficient patient following adenoviral gene transfer, Molecular Fenetics and Metabolism 80, pages 148-158, report on the first death of a patient who received a tDNA encoding the human omithine transcarbamylase (OTC) via the human adenovirus type 5, by means of infusions via the hepatic artery using a femoral catheter. Since this case relates to a death of a patient which could for the first time not clearly be attributed to the basic disease but to the performed somatic gene therapy, a huge number of control experiments have been performed perimortally and postmortally. In the course of the infection phase, i.e. within the first 8 hours following the infusion the vector could be detected in the peripheral blood of the patient, whereas the authors could neither find such vector in the blood nor in the urine, stool nor in the nasal liquid already one day after the infusion, but only postmortal tDNA could be detected in the different organs; cf. page 155, left column, first and second paragraph.

Against this background it could not be expected that tDNA could in fact be found in non-bioptic samples of a living being transfected with said tDNA after the infection phase has been completed. This however could be surprisingly shown by the inventor, where it was found that in such non-bioptic samples the tDNA is highly diluted in comparison to genomic DNA (gDNA). It is therefore assumed that the tDNA has so far not been found in non-bioptic samples due to its low concentration.

The tDNA is detected either via viral segments of the vector or segments of the coding sequence.

The withdrawal of small amounts of non-bioptic samples is absolutely sufficient to perform the method according to the invention in order to enable a reliable detection. For this reason, the method is also suitable for the detection of gene doping, where an analysis is performed for the detection of such tDNA which encodes doping-relevant genes, but also for a detection of such a tDNA which has been introduced into a living being within the scope of a gene therapy.

The object underlying the invention is herewith fully achieved. Especially such a method is provided which avoids an unacceptable invasive intervention in the integrity of the living being to be analyzed.

According to the invention it is preferred if the non-bioptic sample is a blood sample.

The inventor has realized that the tDNA can be found in blood in sufficient amounts and therefore blood is an especially appropriate non-bioptic sample. The exact causes for the presence of tDNA in blood are not known in detail. It is assumed that the transformed cells partially undergo cellular death resulting in a release of intact or fragmented tDNA of preferably >100 and <1000 bp into the peripheral blood. On that occasion it was found out that the tDNA can be present in soluble form but also in the interior of blood cells and possibly packaged into lysosomes.

It is preferred if step (2) comprises the following steps: (2.1) isolation of genetic material contained in the biological sample, and (2.2) performance of a polymerase chain reaction (PCR) with the isolated genetic material, and if step (3) comprises the following step: (3.1) correlation of the obtainment of an amplificate in step (2.2) with a positive detection of tDNA in the living being.

By means of a selective PCR which either amplifies viral segments or the coding sequence of the tDNA itself, the latter can be strongly amplified and, in spite of its high dilution in relation to gDNA, it can be detected in a reliable manner by methods for the visualization of nucleic acids which are well-known in the art, such as electrophoresis or staining with ethidium bromide. In doing so the inventor succeeded in increasing the sensitivity of the method according to the invention resulting in a detection of one tDNA molecule in the background of up to 5 millions of gDNA molecules.

It is preferred if in step (2.2) such a PCR primer pair is used where the first PCR primer can hybridize at stringent conditions to a first exon on the first strand of the tDNA, and the second PCR primer can hybridize at stringent conditions to a second exon on the strand of the tDNA which is complementary to the first strand, which second exon is positioned downstream of the first exon.

This method takes advantage of the finding of the inventor that tDNA in contrast to gDNA is largely or preferably completely intron-free. If for example the sense PCR primer is selected that it can bind to the first exon (E1) of the first strand of the tDNA, the antisense PCR primer is selected in such a manner that it binds 3′-wards to the second strand of the tDNA in the subsequent second exon (E2), which results on the level of the tDNA in an amplification of a relatively short segment of preferably 50 to 400 bp, whereas on the level of gDNA which encodes the corresponding gene, a distinctly longer segment of for example about 1.000 to >10.000 bp is amplified since on the gDNA between E1 and E2 an intron is located which is co-amplified. The presence of tDNA in the blood sample can then be detected on account of the reduced length of the tDNA amplificate over the length of the gDNA amplificate.

According to the invention stringent conditions refer to such reaction conditions where only nucleic acids can hybridize with each other which comprise high complementarity or preferably perfect complementarity on the basis of the nucleotides.

For the method according to the invention it is preferred if in step (2.2) at least one of the two PCR primers is designed like that it is capable to hybridize with a first segment to a first exon of the tDNA and simultaneously with a second segment to a second exon of the tDNA (intron-spanning PCR primer).

This measure has the advantage that the sensitivity of the method according to the invention is once more increased. Intron-spanning PCR primers refer to such primers which can only bind to a segment of the tDNA which contains at least two adjacent exons, i.e. the interface of at least two exons. Such PCR primers are “intron-spanning” since they are not able to hybridize to intron sequences which are located between two exons like e.g. on the gDNA. For example, a 5′-wards located sequence segment of the intron-spanning PCR primer hybridizes to more or less complementary sequence segments which belong to a first exon (for example E1) on the tDNA, a 3′-wards located sequence segment of the intron-spanning primer hybridizes to more or less complementary segments which belong to a second exon (for example E2) on the tDNA. Such an intron-spanning PCR primer can, therefore, only bind under stringent conditions to the intron-free tDNA in a stable manner, however not to gDNA, since this is prevented by the intron sequences which are located between the exons on the gDNA. According to this embodiment it is sufficient if one of the two primers is designed as an intron-spanning primer, whereas the sensitivity is further increased if both primers are intron-spanning primers.

According to a preferred embodiment of the method according to the invention the at least one intron-spanning PCR primer is designed in such a manner that it can hybridize to such regions of said first and said second exons on said tDNA, which are conserved among splice variants of such genes from which the coding sequence of the tDNA derives.

This method has the particular advantage that with such a primer various splice variants of the transgene in question can be detected which results in a further increase of the method. It is known that e.g. the human growth hormone (hGH) has several splice variants which in part differ from each other in their sequences, however are comparably functional and therefore could be used within the scope of a somatic gene therapy or gene doping, respectively. However, such splice variants comprise conserved segments with largely identical nucleotides in corresponding positions. In this connection the identity among these splice variants is preferably 90%, further preferred 95% and highly preferred 100%. It is also known that for an achievement of a doping-relevant effect very often only a specific segment of the peptide hormone is required but not necessarily the peptide hormone in total, consequently, according to the invention, regions of the exons on said tDNA which are conserved among splice variants, i.e. conserved regions, refer to such regions which are compellingly necessary to obtain the wanted effect and, therefore, have to be present in the transgene. Such conserved regions can also be found in the transition area of two adjacent exons, wherein a first part of the conserved region is located 5′-wards in a first exon (e.g. E1) of the tDNA and a second part of said conserved segment is located 3′-wards in an adjacent second exon (e.g. E2). This measure enables the detection of almost all theoretically possible splice variants encoded by the tDNA by only one or a few number of PCRs.

According to a preferred embodiment of the invention in step (2.2) the PCR is performed as so-called “nested” PCR comprising a pre-PCR and a subsequent secondary PCR.

This measure has the particular advantage that the specificity and efficiency of the performed PCR is further increased. In a first PCR round by a so-called pre-PCR a first template is amplified in few cycles. The primers are selected in such a way that the latter are spaced by a comparable large distance, i.e. the sense PCR primer hybridizes for example in the transition area of exon 1 (E1) and exon 2 (E2), the antisense PCR primer, however, hybridizes to the transition area of exon 5 (E5) and exon 6 (E6). The amplificate of this pre-PCR is then amplified by a further PCR round, the so-called secondary PCR or post-PCR, by use of a new PCR primer pair. The new PCR primers are located inwards in relation to the first PCR primers so that in this second step only the specific tDNA segments of the pre-PCR are amplified. The sense PCR primer of the secondary PCR now binds to the transition area of exon 2 (E2) to exon 3 (E3) and the antisense PCR primer binds to the transition area of exon 3 (E3) to exon 4 (E4). The secondary PCR can also be performed if just one primer is located inwards in relation to the first PCR primers, for example only the antisense PCR primer, which binds to the transition area of exon 4 (E4) to exon 5 (E5). By doing so, the efficiency of the method according to the invention is remarkably increased.

It is preferred if the method according to the invention is applied to the detection of such a tDNA which encodes doping-relevant proteins or such proteins which are of relevance for a somatic gene therapy.

By this measure, a reliable and easy-to-handle and little invasive method is provided which is suitable as a highly sensitive standard method for the detection of gene doping or a performed somatic gene therapy. The relevant proteins are preferably selected from the group consisting of erythropoietin (EPO), growth hormone 1 (GH1), growth hormone 2 (GH2), insulin-like growth factor-1 (IGF1), insulin-like growth factor-2 (IGF2), myogenin, peroxisome proliferator-activated receptor delta (PPARd), calcineurin A alpha, vascular-endothelial growth factor (VEGF), chorionic somatomammo-tropin hormone 1 (CSH1), chorionic somatomammo-tropin hormone 1/2 (CSH1/CSH2), chorionic somatomammo-tropin hormone 2 (CSH2), chorionic somatomammo-tropin hormone-like 1 (CSHL1), and myostatin inhibitor. It is further preferred if each of such proteins are of human origin.

This measure has the advantage that the method according to the invention is now suitable for the detection of the most important gene therapy- and doping-relevant proteins. The human variants of all of these proteins are sequenced and the amino acid and nucleotide sequences can be obtained from public databases, such as the NCBI database. Accordingly, the corresponding preferred intron-spanning primers can be easily designed by a skilled person.

A further subject-matter of the present invention relates to a kit which comprises a manual for performing the method according to the invention and, if applicable, reagents, solutions, reaction vials and further beneficial substances and objects.

This measure has the advantage that all information, reagents and reaction vials are prepackaged, what enables the performance of a test for genetic manipulation also outside of a clinical laboratory by semi-skilled staff. Such a test kit for gene modification can, for example, contain a set of different PCR primers for different tDNAs, sufficient amounts of taq-DNA polymerase, nucleotide triphosphates, salts like magnesium chloride, reaction buffer, pure water, etc. The kit may further contain syringes, cannulas and other objects for taking of blood sample, pipettes, reaction vials, coolants and, if applicable, also a device for performing a PCR such as a thermocycler. This assembly of a manual for performing the method according to the invention as well as the required utensils ensures the proper performance of the method and prevents false-negative and false-positive results.

It goes without saying that the before-mentioned features and the features to be described in the following cannot only be used in the identified combinations but also in different combinations or in isolated form, without departing the scope of the present invention.

The present invention is now described in more detail by means of embodiments which are of pure illustrative character and do not limit the scope of the invention. Reference is made to the enclosed figures:

### BRIEF DESCRIPTION OF THE DRAWINGS

FIG. 1 shows (A) the problem of the detection of gene doping or of a performed somatic gene therapy from withdrawn non-bioptic material, (B) the principle of the intron-spanning PCR primers and the principle of the nested PCR;

FIG. 2 shows the principle of the selection of suitable PCR primers for the detection of tDNA encoding several growth hormones;

FIG. 3 shows the principle of the selection of suitable PCR primers for the detection of tDNA encoding erythropoietin (EPO);

FIG. 4 shows the principle of the selection of suitable PCR primers for the detection of tDNA encoding myostatin inhibitor (GDF8 inhibitor);

FIG. 5 shows the principle of the selection of suitable PCR primers for the detection of tDNA encoding insulin-like growth factor 1 (IGF1);

FIG. 6 shows the principle of the selection of suitable PCR primers for the detection of tDNA encoding insulin-like growth factor 2 (IGF2);

FIG. 7 shows the principle of the selection of suitable PCR primers for the detection of tDNA encoding myogenin (MYOG);

FIG. 8 shows the principle of the selection of suitable PCR primers for the detection of tDNA encoding peroxisome proliferator activated receptor delta (PPARd);

FIG. 9 shows the principle of the selection of suitable PCR primers for the detection of tDNA encoding calcineurin A alpha (PP3CA);

FIG. 10 shows the principle of the selection of suitable PCR primers for the detection of tDNA encoding vascular-endothelial growth factor (VEGF);

FIG. 11 shows the result of the separation of an EPO-tDNA PCR product resulting from the pre-PCR by gel electrophoresis (A) and resulting from the corresponding secondary PCR (B and C) on a 1.5% agarose gel.

### DETAILED DESCRIPTION OF EMBODIMENTS

### Example 1

### Gene Therapeutically and Doping-Relevant Genes

In Table 1 below the most important candidate genes are listed, the gene products of which have already been proven for their doping-relevant or gene therapeutic functionality in animal experiments. Indicated are the name of the gene, the official abbreviation, the chromosomal localization, and in the column NBCI Gene ID the NCBI reference number for the gene. In the column UniProtKB the protein variants and the reference accession numbers of the Swiss Prot Protein database are listed. In the next column the accession number for the NCBI database for each known splice variant is identified, by which the corresponding mRNA sequence can be obtained. On the basis of the mRNA sequence and the corresponding gene sequence which can be obtained via the NCBI reference number of the gene suitable PCR primers for the amplification of the corresponding tDNA can be derived.

In the case of genes which have many alternative splice variants, such as VEGF, or in the case where, beside the alternative splice variants, species-related genes do exist having high conservation between each other and which encode a similar protein, such as GH1, GH2, CSH1, CSH2, CSHL1, the PCR primer has to be designed in such a manner that it hybridizes to the transition areas of two adjacent exons, which are highly conserved among the splice variants (see example 2 below). In such cases for a detection by means of intron-spanning primers frequently only few sequence segments can be used which can be found in all or in many variants.

TABLE 1
List of the most important gene therapeutically- and doping-relevant genes

| | | | | | | | treatment/ |
|---|---|---|---|---|---|---|---|
| | | | | | | | performance |
| | Official | Chromosomal | NCBI Gene | UniProtKB | NCBI mRNA | | enhancing: |
| Gene | abbreviation | localisation | ID | access. No. | access. No. | Remarks | effect |
| Chorionic somato- | CSH1 | 17q23.3 | 1442 | P01243 | NM_001317 | | hyposomia/ |
| mammo-tropin | | | | Q7KZ35 | NM_022641 | | strength |
| hormone 1 | | | | | | | and speed |
| Chorionic somato- | CSH1/CSH2 | 17q23.3 | 1442/1443 | P01243 | CR620501 | This variant uses | hyposomia/ |
| mammo-tropin | | | | | | promoter and first exon | strength |
| hormone 1/2 | | | | | | of CSH1, the other 4 | and speed |
| | | | | | | exons belong to CSH2 | |
| Chorionic somato- | CSH2 | 17q23.3 | 1443 | P01243 | NM_020991 | | hyposomia/ |
| mammo-tropin | | | | Q7KZ35 | NM_022645 | | strength |
| hormone 2 | | | | | | | and speed |
| Chorionic somato- | CSHL1 | 17q23.3 | 1444 | Q14406-1 | NM_022579 | | hyposomia/ |
| mammo-tropin- | | | | Q14406-2 | NM_022581 | | strength |
| hormone-like 1 | | | | | | | and speed |
| Erythropoietin | EPO | 7q22 | 2056 | P01588 | NM_000799 | | anemia/ |
| | | | | | | | endurance |
| Growth differ- | GDF8 | 2q32.2 | 2660 | O14793 | NM_005259 | The bases 956-end of | degenerative |
| entiation factor | | | | | | NM_005259 may be | diseases of the |
| 8 (Myostatin) | | | | | | deleted or modified for | musculature/ |
| | | | | | | gene doping | strength |
| | | | | | | | and speed |
| Growth hormone 1 | GH1 | 17q24.2 | 2688 | P01241 | NM_000515 | | hyposomia/ |
| | | | | P01241-2 | NM_022559 | | strength |
| | | | | P01241-3 | AF185611 | | and speed |
| | | | | P01241-4 | AF110644 | | |
| Growth hormone 2 | GH2 | 17q24.2 | 2689 | P01242 | NM_002059 | | hyposomia/ |
| | | | | O14643 | NM_022556 | | strength |
| | | | | P01242-2 | NM_022557 | | and speed |
| | | | | O14644 | NM_022558 | | |
| Insulin-like | IGF1 | 12q22-q23 | 3479 | P01343 | NM_000618 | | degenerative |
| growth factor 1 | | | | P01343 | M29644 | | diseases of the |
| (Somatomedin C) | | | | Q14620 | M37484 | | musculature/ |
| | | | | P05019 | M11568 | | strength |
| | | | | | | | and speed |
| Insulin-like | IGF2 | 11p15.5 | 3481 | P01344 | NM_000612 | | degenerative |
| growth factor 2 | | | | | | | diseases of the |
| | | | | | | | musculature/ |
| | | | | | | | strength |
| | | | | | | | and speed |
| Myogenin | MYOG | 1q31-q41 | 4656 | P15173 | NM_002479 | | degenerative |
| | | | | | | | diseases of the |
| | | | | | | | musculature/ |
| | | | | | | | strength |
| | | | | | | | speed and |
| | | | | | | | endurance |
| Peroxisome | PPARd | 6p21.2-p21.1 | 5467 | Q03181 | NM_006238 | | degenerative |
| proliferator- | | | | Q03181-2 | NM_177435 | | diseases of the |
| activated | | | | | | | musculature/ |
| receptor delta | | | | | | | strength, |
| | | | | | | | speed and |
| | | | | | | | endurance |
| Protein | PPP3CA | | 5530 | Q08209 | NM_000944 | The bases 1802-1868 of | degenerative |
| phosphatase 3, | | | | Q8TAW9 | BC025714 | NM_00944 (inhibitory | diseases of the |
| catalytic subunit, | | | | | | domain) may be deleted | musculature/ |
| alpha isoform | | | | | | for gene doping | strength |
| (calcineurin A | | | | | | | and speed |
| alpha) | | | | | | | |
| Vascular | VEGF | 6p12 | 7422 | Q96FD9 | NM_001025370 | | cardiac infarction |
| endothelial | | | | Q96FD9 | NM_001033756 | | and coronary heart |
| growth factor | | | | P15692-2 | NM_003376 | | diseases/strength, |
| | | | | P15692 | NM_001025366 | | speed and endurance |
| | | | | P15692-3 | NM_001025367 | | |
| | | | | P15692-4 | NM_001025368 | | |
| | | | | Q96FD9 | NM_001025369 | | |
| | | | | P15692-2 | M27281 | | |
| | | | | P15692 | S85192 | | |

### Example 2

### The Principle of the Intron-Spanning PCR Primers

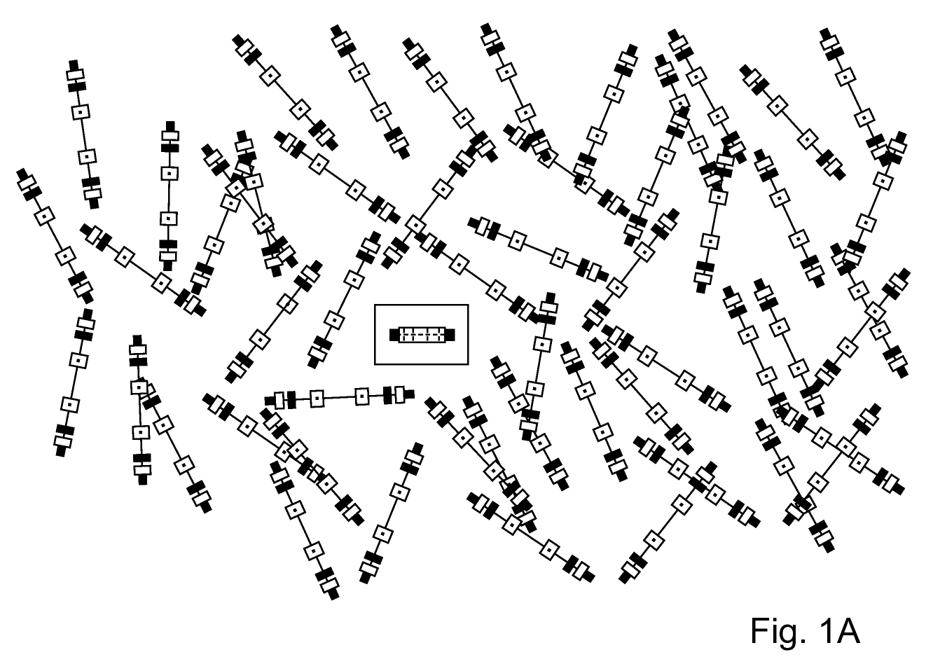

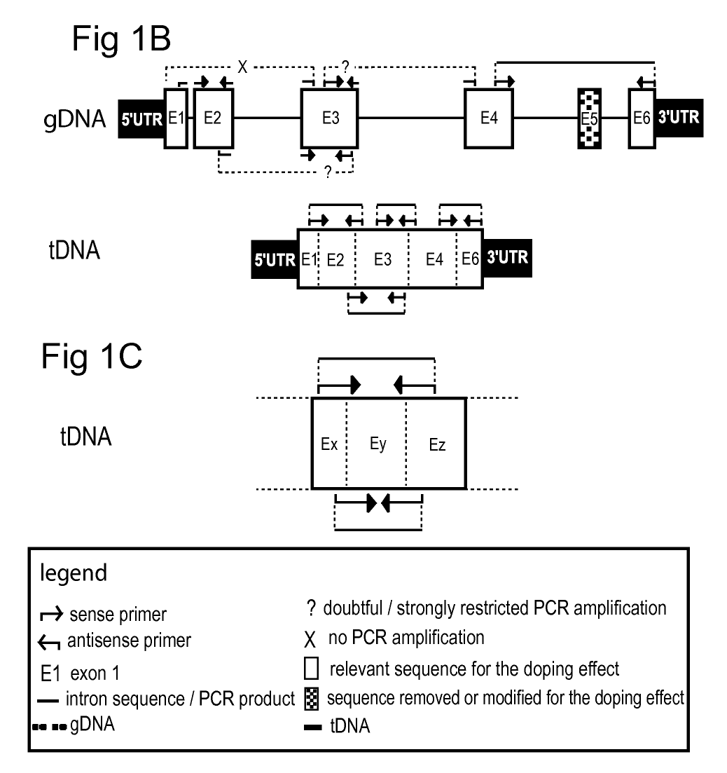

FIG. 1A shows schematically the problem of the detection of gene doping or gene therapy in non-bioptic material. The transgenic DNA (tDNA) prevails in a highly diluted manner in relation to the genomic DNA (gDNA), what basically makes the detection of a performed genetic modification difficult. In 50 μg of isolated total DNA from non-bioptic material, such as blood, stool or urine, on the average about 10⁷ copies of gDNA can be found. To detect one single copy of tDNA in 50 μg of isolated total DNA the tDNA is preferably to be amplified by the factor 10¹¹.

FIG. 1B shows the principle of the intron-spanning PCR primers. The gDNA comprises 6 exons (E1 to E6) with inter-adjacent introns, whereas the tDNA is intron-free and does also not contain E5 which is not required in the organism for the desired doping effect. The black primer pair enables the highest specificity for the PCR amplification of the tDNA also at a high dilution by gDNA, since both primers are primer-internal intron-spanning primers. The black sense-PCR primer hybridizes to the transition area of exon 1 (E1) and exon 2 (E2), whereas the antisense PCR primers hybridizes to the transition area of exon 2 (E2) and exon 3 (E3). In the following, such a primer pair is referred to as “bilateral intron-spanning primer pair”. Bilateral intron-spanning primer pairs can span two introns or more than two introns.

Both of the dark grey primers are “unilateral intron-spanning primer pairs”. In the upper dark grey primer pair only the antisense PCR primer is designed as an intron-spanning primer which hybridizes to the transition area of E3 and E4. In the lower dark grey primer pair only the sense PCR primer is designed as an intron-spanning primer and hybridizes to the transition area of E2 and E3. In both cases for the unilateral intron-spanning primer pairs the sensitivity and specificity of the tDNA amplification is slightly worse than for the black bilateral intron-spanning primer pair since at least one primer [dark grey (above): sense PCR primer; dark grey (below): antisense PCR primer] exhibits full affinity to the excess gDNA.

The dark grey primer pair is a so-called “primer external intron-spanning primer pair”. Each of both primers hybridizes exclusively to one exon but not simultaneously to two exons, i.e. not to transition areas of two different exons. The sense PCR primer hybridizes exclusively to E4 and the antisense PCR primer hybridizes exclusively to E6. Also by this measure, tDNA can be detected since the products or amplificates, respectively, of gDNA and tDNA differ in their sizes. With this approach using a primer external intron-spanning primer pair, the sensitivity and the specificity are however worse than the approach using unilateral or bilateral intron-spanning primer pairs due to the high dilution of the tDNA and due to the PCR product which results from the gDNA.

The highest sensitivity is obtained by performing a pre-PCR with the sense primer of the primer pair 1 and the antisense primer of the primer pair 3. After this pre-PCR a secondary PCR is performed with the diluted pre-PCR amplificate and the use of more inwardly located primer pairs, i.e. the primer pair 1, primer pair 2 and primer pair 3. This measure is also referred to as “nested” PCR.

FIG. 1C shows the principle of a nested PCR. As mentioned above, for an optimum detection the tDNA of 50 μg preferably isolated total DNA is amplified by the factor 10¹¹, since 10¹¹ copies of a 400 bp DNA have a weight of about 50 ng which, inter alia, are sufficient for the sequencing of a PCR product. In order to obtain a 10¹¹-fold amplification, consequently about 37 optimum PCR cycles are required. Since the PCR is an enzymatic reaction which naturally is subject to saturation, 37 optimum PCR cycles cannot be reached in one PCR operation. In order to reach a maximum sensitivity two consecutive PCR operations are performed, wherein the first operation is referred to as pre-PCR and the second operation as secondary PCR. By doing so the diluted PCR amplificate resulting from the pre-PCR is used as a template in the secondary PCR. The number of cycles in the pre- and secondary PCR is between 20 and 35 cycles and varies in dependence of the used primers.

In order to test for tDNA of as much different candidate genes as possible for genetic modifications, in some cases the pre-PCR is performed as a so-called multiplex PCR. In this case in the pre-PCR several primer pairs are used simultaneously to start with a pre-amplification of a broad range of tDNAs. In the secondary PCR gene-specific primers are used to specifically amplify individual tDNA candidates out of the pre-PCR.

To obtain the highest sensitivity in the example shown in FIG. 1C a pre-PCR is performed by using the upper black primer pair. After this pre-PCR the diluted pre-PCR amplificate is subjected to a secondary PCR either again with the upper black primer pair or with the below black primer pair (so-called “nested” PCR). The secondary PCR in form of a nested secondary PCR results therefore in a smaller amplificate in comparison to the pre-PCR. In this case, the primers of this secondary PCR could also span a small number of introns.

### Example 3

### Primer Design

3.1 Primers for the Detection of Gene Therapy or Doping by Means of a tDNA Encoding the Growth Hormone (GH), Chorionic Somatomammo-Tropin Hormone (CSH) and Chorionic Somatomammo-Tropin Hormone-Like (CSHL) Genes

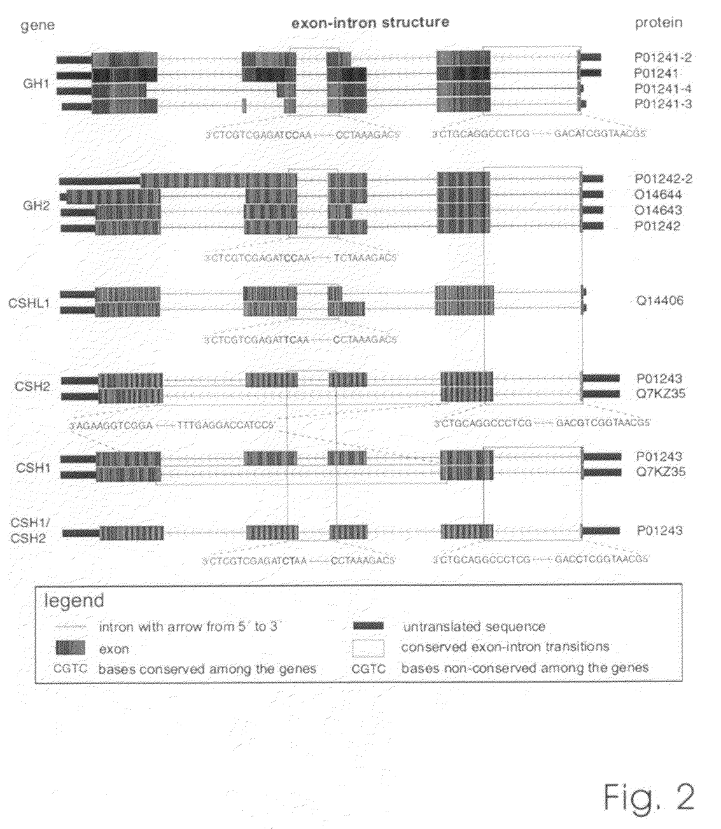

FIG. 2 illustrates in a diagram the protein-encoding reference sequences of the five growth hormone sequences which are located in the so-called growth hormone locus 17q23.3. The exon-intron structure is shown for all 15 reference mRNA sequences of the growth hormone. All five genes share 90% sequence homology. By multiple sequence alignments three exon-intron transitions (boxes) have been determined which comprise a sufficient homology to detect all candidates in a sensitive manner and by means of a manageable number of PCRs. In this case, three sequence segments have been chosen for the design of sense primers and five sequence segments have been selected for the design of antisense primers. The primers can be used altogether at 0.2 μM each in a multiplex pre-PCR. Subsequently, seven PCRs can be performed for the different gene-specific detection. The CSH1/CSH2 hybrid consists of the exon 1 of the CSH1 locus and the exons 2-4 of the CSH2 locus.

Different mRNAs splice variants of the growth hormone locus were compared with each other to determine highly conserved parts in the transition areas of two exons to design corresponding PCR primers. In each case, the total mRNA is shown, the selected sense primer is shown in bold letters and the antisense primer is underlined.

a) GH1 primer:
>gi|20809248|ref|NM_000515.3| Homo sapiens growth
hormone 1 (GH1), transcript variant 1, mRNA
encoding P01241
AGGATCCCAAGGCCCAACTCCCCGAACCACTCAGGGTCCTGTGGACAGCT
CACCTAGCTGCAATGGCTACAGGCTCCCGGACGTCCCTGCTCCTGGCTTT
TGGCCTGCTCTGCCTGCCCTGGCTTCAAGAGGGCAGTGCCTTCCCAACCA
TTCCCTTATCCAGGCTTTTTGACAACGCTATGCTCCGCGCCCATCGTCTG
CACCAGCTGGCCTTTGACACCTACCAGGAGTTTGAAGAAGCCTATATCCC
AAAGGAACAGAAGTATTCATTCCTGCAGAACCCCCAGACCTCCCTCTGTT
TCTCAGAGTCTATTCCGACACCCTCCAACAGGGAGGAAACACAACAGAAA
TCCAACCTAGAGCTGCTCCGCATCTCCCTGCTGCTCATCCAGTCGTGGCT
GGAGCCCGTGCAGTTCCTCAGGAGTGTCTTCGCCAACAGCCTGGTGTACG
GCGCCTCTGACAGCAACGTCTATGACCTCCTAAAGGACCTAGAGGAAGGC
ATCCAAACGCTGATGGGGAGGCTGGAAGATGGCAGCCCCCGGACTGGGCA
GATCTTCAAGCAGACCTACAGCAAGTTCGACACAAACTCACACAACGATG
ACGCACTACTCAAGAACTACGGGCTGCTCTACTGCTTCAGGAAGGACATG
GACAAGGTCGAGACATTCCTGCGCATCGTGCAGTGCCGCTCTGTGGAGGG
CAGCTGTGGCTTCTAGCTGCCCGGGTGGCATCCCTGTGACCCCTCCCCAG
TGCCTCTCCTGGCCCTGGAAGTTGCCACTCCAGTGCCCACCAGCCTTGTC
CTAATAAAATTAAGTTGCATCA
>gi|20809249|ref|NM_022559.2| Homo sapiens growth
hormone 1 (GH1), transcript variant 2, mRNA
encoding P01241-2
AGGATCCCAAGGCCCAACTCCCCGAACCACTCAGGGTCCTGTGGACAGCT
CACCTAGCTGCAATGGCTACAGGCTCCCGGACGTCCCTGCTCCTGGCTTT
TGGCCTGCTCTGCCTGCCCTGGCTTCAAGAGGGCAGTGCCTTCCCAACCA
TTCCCTTATCCAGGCTTTTTGACAACGCTATGCTCCGCGCCCATCGTCTG
CACCAGCTGGCCTTTGACACCTACCAGGAGTTTAACCCCCAGACCTCCCT
CTGTTTCTCAGAGTCTATTCCGACACCCTCCAACAGGGAGGAAACACAAC
AGAAATCCAACCTAGAGCTGCTCCGCATCTCCCTGCTGCTCATCCAGTCG
TGGCTGGAGCCCGTGCAGTTCCTCAGGAGTGTCTTCGCCAACAGCCTGGT
GTACGGCGCCTCTGACAGCAACGTCTATGACCTCCTAAAGGACCTAGAGG
AAGGCATCCAAACGCTGATGGGGAGGCTGGAAGATGGCAGCCCCCGGACT
GGGCAGATCTTCAAGCAGACCTACAGCAAGTTCGACACAAACTCACACAA
CGATGACGCACTACTCAAGAACTACGGGCTGCTCTACTGCTTCAGGAAGG
ACATGGACAAGGTCGAGACATTCCTGCGCATCGTGCAGTGCCGCTCTGTG
GAGGGCAGCTGTGGCTTCTAGCTGCCCGGGTGGCATCCCTGTGACCCCTC
CCCAGTGCCTCTCCTGGCCCTGGAAGTTGCCACTCCAGTGCCCACCAGCC
TTGTCCTAATAAAATTAAGTTGCATCA
>gi|9963798|gb|AF185611.1|AF185611 Homo sapiens
growth hormone variant (GHV) mRNA, complete coding
sequence (cds) encoding P01241-3
ACAGCTCACCTAGCTGCAATGGCTACAGGCTCCCGGACGTCCCTGCTCCT
GGCTTTTGGCCTGCTCTGCCTGCCCTGGCTTCAAGAGGGCAGTGCCTTCC
CAACCATTCCCTTATCCAGGCTTTTTGACAACGCTATGCTCCGCGCCCAT
CGTCTGCACCAGCTGGCCTTTGACACCTACCAGGAGTTTGAAGAAGCCTA
TATCCCAAAGGAACAGAAGTATTCATTCCTGCAGAACCCCCAGACCTCCC
TCTGTTTCTCAGAGTCTATTCCGACACCCTCCAACAGGGAGGAAACACAA
CAGAAATCCAACCTAGAGCTGCTCCGCATCTCCCTGCTGCTCATCCAAAC
GCTGATGGGGAGGCTGGAAGATGGCAGCCCCCGGACTGGGCAGATCTTCA
AGCAGACCTACAGCAAGTTCGACACAAACTCACACAACGATGACGCACTA
CTCAAGAACTACGGGCTGCTCTACTGCTTCAGGAAGGACATGGACAAGGT
CGAGACATTCCTGCGCATCGTGCAGTGCCGCTCTGTGGAGGGCAGCTGTG
GCTTCTAGCTGCCCGGGTGGCATCCCTGTGACCCCTCCCCAGTGCCTCTC
CTGGCCCTGGAAGTTGCCACTCCAGTGCCCACCAGCCTTGTCCTAATA
>gi|5730477|gb|AF110644.1|AF110644 Homo sapiens
growth hormone splice variant mRNA, complete cds
encoding P01241-4
CCTAGCTGCAATGGCTACAGGCTCCCGGACGTCCCTGCTCCTGGCTTTTG
GCCTGCTCTGCCTGCCCTGGCTTCAAGAGGGCAGTGCCTTCCCAACCATT
CCCTTATCCAGGCTTTTTGACAACGCTATGCTCCGCGCCCATCGTCTGCA
CCAGCTGGCCTTTGACACCTACCAGGAGTTTGAAGAAGCCTATATCCCAA
AGGAACAGAAGTATTCATTCCTGCAGAACCCCCAGACCTCCCTCTGTTTC
TCAGAGTCTATTCCGACACCCTCCAACAGGGAGGAAACACAACAGAAATC
CAACCTAGAGCTGCTCCGCATCTCCCTGCTGCTCATCCAGTCGTGGCTGG
AGCCCGTGCAGATCTTCAAGCAGACCTACAGCAAGTTCGACACAAACTCA
CACAACGATGACGCACTACTCAAGAACTACGGGCTGCTCTACTGCTTCAG
GAAGGACATGGACAAGGTCGAGACATTCCTGCGCATCGTGCAGTGCCGCT
CTGTGGAGGGCAGCTGTGGCTTCTAGCTGCCCGGGTGGCATCCCTGTGAC
CCCTCCCCAGTGCCTCTCCTGGCCCTGGAAGTTGCCACTCCAGTGCCCAC
CAGCCTTGTCCTAATAAAATTAAGTTGCATCAAAAAAAAAAA
b) GH2 primer
>gi|20809256|ref|NM_002059.3| Homo sapiens growth
hormone 2 (GH2), transcript variant 1, mRNA
encoding P01242
AGGATCCCAAGGCCCAACTCCCCGAACCACTCAGGGTCCTGTGGACAGCT
CACCTAGCGGCAATGGCTGCAGGCTCCCGGACGTCCCTGCTCCTGGCTTT
TGGCCTGCTCTGCCTGTCCTGGCTTCAAGAGGGCAGTGCCTTCCCAACCA
TTCCCTTATCCAGGCTTTTTGACAACGCTATGCTCCGCGCCCGTCGCCTG
TACCAGCTGGCATATGACACCTATCAGGAGTTTGAAGAAGCCTATATCCT
GAAGGAGCAGAAGTATTCATTCCTGCAGAACCCCCAGACCTCCCTCTGCT
TCTCAGAGTCTATTCCAACACCTTCCAACAGGGTGAAAACGCAGCAGAAA
TCTAACCTAGAGCTGCTCCGCATCTCCCTGCTGCTCATCCAGTCATGGCT
GGAGCCCGTGCAGCTCCTCAGGAGCGTCTTCGCCAACAGCCTGGTGTATG
GCGCCTCGGACAGCAACGTCTATCGCCACCTGAAGGACCTAGAGGAAGGC
ATCCAAACGCTGATGTGGAGGCTGGAAGATGGCAGCCCCCGGACTGGGCA
GATCTTCAATCAGTCCTACAGCAAGTTTGACACAAAATCGCACAACGATG
ACGCACTGCTCAAGAACTACGGGCTGCTCTACTGCTTCAGGAAGGACATG
GACAAGGTCGAGACATTCCTGCGCATCGTGCAGTGCCGCTCTGTGGAGGG
CAGCTGTGGCTTCTAGCTGCCCGGGTGGCATCCCTGTGACCCCTCCCCAG
TGCCTCTCCTGGTCGTGGAAGGTGCTACTCCAGTGCCCACCAGCCTTGTC
CTAATAAAATTAAGTTGCATC
>gi|20809254|ref|NM_022557.2| Homo sapiens growth
hormone 2 (GH2), transcript variant 2, mRNA
encoding P01242-2
AGGATCCCAAGGCCCAACTCCCCGAACCACTCAGGGTCCTGTGGACAGCT
CACCTAGCGGCAATGGCTGCAGGCTCCCGGACGTCCCTGCTCCTGGCTTT
TGGCCTGCTCTGCCTGTCCTGGCTTCAAGAGGGCAGTGCCTTCCCAACCA
TTCCCTTATCCAGGCTTTTTGACAACGCTATGCTCCGCGCCCGTCGCCTG
TACCAGCTGGCATATGACACCTATCAGGAGTTTGAAGAAGCCTATATCCT
GAAGGAGCAGAAGTATTCATTCCTGCAGAACCCCCAGACCTCCCTCTGCT
TCTCAGAGTCTATTCCAACACCTTCCAACAGGGTGAAAACGCAGCAGAAA
TCTAACCTAGAGCTGCTCCGCATCTCCCTGCTGCTCATCCAGTCATGGCT
GGAGCCCGTGCAGCTCCTCAGGAGCGTCTTCGCCAACAGCCTGGTGTATG
GCGCCTCGGACAGCAACGTCTATCGCCACCTGAAGGACCTAGAGGAAGGC
ATCCAAACGCTGATGTGGGTGAGGGTGGCACCAGGGATCCCCAATCCTGG
GGCCCCACTGGCTTCCAGGGACTGGGGAGAGAAACACTGCTGCCCTCTTT
TTAGCAGTCAGGCGCTGACCCAAGAGAACTCACCGTATTCTTCATTTCCC
CTCGTGAATCCTCCAGGCCTTTCTCTACAACCTGGAGGGGAGGGAGGAAA
ATGGATGAATGAGAGAGGGAGGGAACAGTGCCCAAGCGCTTGGCCTCTCC
TTCTCTTCCTTCACTTTGCAGAGGCTGGAAGATGGCAGCCCCCGGACTGG
GCAGATCTTCAATCAGTCCTACAGCAAGTTTGACACAAAATCGCACAACG
ATGACGCACTGCTCAAGAACTACGGGCTGCTCTACTGCTTCAGGAAGGAC
ATGGACAAGGTCGAGACATTCCTGCGCATCGTGCAGTGCCGCTCTGTGGA
GGGCAGCTGTGGCTTCTAGCTGCCCGGGTGGCATCCCTGTGACCCCTCCC
CAGTGCCTCTCCTGGTCGTGGAAGGTGCTACTCCAGTGCCCACCAGCCTT
GTCCTAATAAAATTAAGTTGCATCATTT
>gi|20809255|ref|NM_022558.2| Homo sapiens growth
hormone 2 (GH2), transcript variant 3, mRNA
encoding O14644
AGGATCCCAAGGCCCAACTCCCCGAACCACTCAGGGTCCTGTGGACAGCT
CACCTAGCGGCAATGGCTGCAGGCTCCCGGACGTCCCTGCTCCTGGCTTT
TGGCCTGCTCTGCCTGTCCTGGCTTCAAGAGGGCAGTGCCTTCCCAACCA
TTCCCTTATCCAGGCTTTTTGACAACGCTATGCTCCGCGCCCGTCGCCTG
TACCAGCTGGCATATGACACCTATCAGGAGTTTGAAGAAGCCTATATCCT
GAAGGAGCAGAAGTATTCATTCCTGCAGAACCCCCAGACCTCCCTCTGCT
TCTCAGAGTCTATTCCAACACCTTCCAACAGGGTGAAAACGCAGCAGAAA
TCTAACCTAGAGCTGCTCCGCATCTCCCTGCTGCTCATCCAGTCATGGCT
GGAGCCCGTGCAGCTCCTCAGGAGCGTCTTCGCCAACAGCCTGGTGTATG
GCGCCTCGGACAGCAACGTCTATCGCCACCTGAAGGACCTAGAGGAAGGC
ATCCAAACGCTGATAGGCTGGAAGATGGCAGCCCCCGGACTGGGCAGATC
TTCAATCAGTCCTACAGCAAGTTTGACACAAAATCGCACAACGATGACGC
ACTGCTCAAGAACTACGGGCTGCTCTACTGCTTCAGGAAGGACATGGACA
AGGTCGAGACATTCCTGCGCATCGTGCAGTGCCGCTCTGTGGAGGGCAGC
TGTGGCTTCTAGCTGCCCGGGTGGCATCCCTGTGACCCCTCCCCAGTGCC
TCTCCTGGTCGTGGAAGGTGCTACTCCAGTGCCCACCAGCCTTGTCCTAA
TAAAATTAAGTTGCATCAT
>gi|20809253|ref|NM_022556.2| Homo sapiens growth
hormone 2 (GH2), transcript variant 4, mRNA
encoding O14643
AGGATCCCAAGGCCCAACTCCCCGAACCACTCAGGGTCCTGTGGACAGCT
CACCTAGCGGCAATGGCTGCAGGCTCCCGGACGTCCCTGCTCCTGGCTTT
TGGCCTGCTCTGCCTGTCCTGGCTTCAAGAGGGCAGTGCCTTCCCAACCA
TTCCCTTATCCAGGCTTTTTGACAACGCTATGCTCCGCGCCCGTCGCCTG
TACCAGCTGGCATATGACACCTATCAGGAGTTTAACCCCCAGACCTCCCT
CTGCTTCTCAGAGTCTATTCCAACACCTTCCAACAGGGTGAAAACGCAGC
AGAAATCTAACCTAGAGCTGCTCCGCATCTCCCTGCTGCTCATCCAGTCA
TGGCTGGAGCCCGTGCAGCTCCTCAGGAGCGTCTTCGCCAACAGCCTGGT
GTATGGCGCCTCGGACAGCAACGTCTATCGCCACCTGAAGGACCTAGAGG
AAGGCATCCAAACGCTGATGTGGAGGCTGGAAGATGGCAGCCCCCGGACT
GGGCAGATCTTCAATCAGTCCTACAGCAAGTTTGACACAAAATCGCACAA
CGATGACGCACTGCTCAAGAACTACGGGCTGCTCTACTGCTTCAGGAAGG
ACATGGACAAGGTCGAGACATTCCTGCGCATCGTGCAGTGCCGCTCTGTG
GAGGGCAGCTGTGGCTTCTAGCTGCCCGGGTGGCATCCCTGTGACCCCTC
CCCAGTGCCTCTCCTGGTCGTGGAAGGTGCTACTCCAGTGCCCACCAGCC
TTGTCCTAATAAAATTAAGTTGCATC
c) CSH1-Primer
>gi|20819954|ref|NM_001317.3| Homo sapiens
chorionic somatomammo-tropin hormone 1 (placenta
lactogen) (CSH1), transcript variant 1, mRNA
encoding P01243
GCAGGGAGAGAGAACTGGCCAGGGTATAAAAAGGGCCCACAAGAGACCGG
CTCTAGGATCCCAAGGCCCAACTCCCCGAACCACTCAGGGTCCTGTGGAC
AGCTCACCTAGTGGCAATGGCTCCAGGCTCCCGGACGTCCCTGCTCCTGG
CTTTTGCCCTGCTCTGCCTGCCCTGGCTTCAAGAGGCTGGTGCCGTCCAA
ACCGTTCCGTTATCCAGGCTTTTTGACCACGCTATGCTCCAAGCCCATCG
CGCGCACCAGCTGGCCATTGACACCTACCAGGAGTTTGAAGAAACCTATA
TCCCAAAGGACCAGAAGTATTCATTCCTGCATGACTCCCAGACCTCCTTC
TGCTTCTCAGACTCTATTCCGACACCCTCCAACATGGAGGAAACGCAACA
GAAATCCAATCTAGAGCTGCTCCGCATCTCCCTGCTGCTCATCGAGTCGT
GGCTGGAGCCCGTGCGGTTCCTCAGGAGTATGTTCGCCAACAACCTGGTG
TATGACACCTCGGACAGCGATGACTATCACCTCCTAAAGGACCTAGAGGA
AGGCATCCAAACGCTGATGGGGAGGCTGGAAGACGGCAGCCGCCGGACTG
GGCAGATCCTCAAGCAGACCTACAGCAAGTTTGACACAAACTCGCACAAC
CATGACGCACTGCTCAAGAACTACGGGCTGCTCTACTGCTTCAGGAAGGA
CATGGACAAGGTCGAGACATTCCTGCGCATGGTGCAGTGCCGCTCTGTGG
AGGGCAGCTGTGGCTTCTAGGTGCCCGAGTAGCATCCTGTGACCCCTCCC
CAGTGCCTCTCCTGGCCCTGAAGGTGCCACTCCAGTGCCCACCAGCCTTG
TCCTAATAAAATTAAGTTGTATCATTTCA
>gi|20819966|ref|NM_022641.2| Home sapiens
chorionic somatomammo-tropin hormone 1 (placenta
lactogen) (CSH1), transcript variant 3, mRNA for
Q7KZ35
GCAGGGAGAGAGAACTGGCCAGGGTATAAAAAGGGCCCACAAGAGACCGG
CTCTAGGATCCCAAGGCCCAACTCCCCGAACCACTCAGGGTCCTGTGGAC
AGCTCACCTAGTGGCAATGGCTCCAGGCTCCCGGACGTCCCTGCTCCTGG
CTTTTGCCCTGCTCTGCCTGCCCTGGCTTCAAGAGGCTGGTGCCGTCCAA
ACCGTTCCGTTATCCAGGCTTTTTGACCACGCTATGCTCCAAGCCCATCG
CGCGCACCAGCTGGCCATTGACACCTACCAGGAGTTTAGGCTGGAAGACG
GCAGCCGCCGGACTGGGCAGATCCTCAAGCAGACCTACAGCAAGTTTGAC
ACAAACTCGCACAACCATGACGCACTGCTCAAGAACTACGGGCTGCTCTA
CTGCTTCAGGAAGGACATGGACAAGGTCGAGACATTCCTGCGCATGGTGC
AGTGCCGCTCTGTGGAGGGCAGCTGTGGCTTCTAGGTGCCCGAGTAGCAT
CCTGTGACCCCTCCCCAGTGCCTCTCCTGGCCCTGAAGGTGCCACTCCAG
TGCCCACCAGCCTTGTCCTAATAAAATTAAGTTGTATCATTTCA
>gi|50501308|emb|CR620501.1| Full length cDNA
clone CS0DI037YC04 of placenta Cot 25-normalized
of homo sapiens (human) encoding P01243
TCAGGGTCCTGTGGACAGCTCACCTAGTGGCAATGGCTCCAGGCTCCCGG
ACGTCCCTGCTCCTGGCTTTTGCCCTGCTCTGCCTGCCCTGGCTTCAAGA
GGCTGGTGCCGTCCAAACCGTTCCGTTATCCAGGCTTTTTGACCACGCTA
TGCTCCAAGCCCATCGCGCGCACCAGCTGGCCATTGACACCTACCAGGAG
TTTGAAGAAACCTATATCCCAAAGGACCAGAAGTATTCATTCCTGCATGA
CTCCCAGACCTCCTTCTGCTTCTCAGACTCTATTCCGACACCCTCCAACA
TGGAGGAAACGCAACAGAAATCCAATCTAGAGCTGCTCCGCATCTCCCTG
CTGCTCATCGAGTCGTGGCTGGAGCCCGTGCGGTTCCTCAGGAGTATGTT
CGCCAACAACCTGGTGTATGACACCTCGGACAGCGATGACTATCACCTCC
TAAAGGACCTAGAGGAAGGCATCCAAACGCTGATGGGGAGGCTGGAAGAC
GGCAGCCGCCGGACTGGGCAGATCCTCAAGCAGACCTACAGCAAGTTTGA
CACAAACTCACACAACCATGACGCACTGCTCAAGAACTACGGGCTGCTCT
ACTGCTTCAGGAAGGACATGGACAAGGTCGAGACATTCCTGCGCATGGTG
CAGTGCCGCTCTGTAGAGGGTAGCTGTGGCTTCTAGGTGCCCGCGTGGCA
TCCTGTGACCGACCCCTCCCCAGTGCCTCTCCTGGCCCTGGAAGGTGCCA
CTCCAGTGCCCATCAGCCTTGTCCTAATAAAATTAAGTTGTATCATTT
d) CSH2 primer
>gi|20819978|ref|NM_020991.3| Homo sapiens
chorionic somatomammo-tropin hormone 2 (CSH2) mRNA
encoding P01243
CAGGGAGAGAGAACTGGCCAGGGTATAAAAAGGGCCCACAAGAGACCGGC
TCTAGGATCCCAAGGCCCAACTCCCCGAACCACTCAGGGTCCTGTGGACA
GCTCACCTAGCGGCAATGGCTGCAGGCTCCCGGACGTCCCTGCTCCTGGC
TTTTGCCCTGCTCTGCCTGCCCTGGCTTCAAGAGGCTGGTGCCGTCCAAA
CCGTTCCGTTATCCAGGCTTTTTGACCACGCTATGCTCCAAGCCCATCGC
GCGCACCAGCTGGCCATTGACACCTACCAGGAGTTTGAAGAAACCTATAT
CCCAAAGGACCAGAAGTATTCATTCCTGCATGACTCCCAGACCTCCTTCT
GCTTCTCAGACTCTATTCCGACACCCTCCAACATGGAGGAAACGCAACAG
AAATCCAATCTAGAGCTGCTCCGCATCTCCCTGCTGCTCATCGAGTCGTG
GCTGGAGCCCGTGCGGTTCCTCAGGAGTATGTTCGCCAACAACCTGGTGT
ATGACACCTCGGACAGCGATGACTATCACCTCCTAAAGGACCTAGAGGAA
GGCATCCAAACGCTGATGGGGAGGCTGGAAGACGGCAGCCGCCGGACTGG
GCAGATCCTCAAGCAGACCTACAGCAAGTTTGACACAAACTCACACAACC
ATGACGCACTGCTCAAGAACTACGGGCTGCTCTACTGCTTCAGGAAGGAC
ATGGACAAGGTCGAGACATTCCTGCGCATGGTGCAGTGCCGCTCTGTAGA
GGGTAGCTGTGGCTTCTAGGTGCCCGCGTGGCATCCTGTGACCGACCCCT
CCCCAGTGCCTCTCCTGGCCCTGGAAGGTGCCACTCCAGTGCCCATCAGC
CTTGTCCTAATAAAATTAAGTTGTATCATTTCA
>gi|20819991|ref|NM_022645.2| Homo sapiens
chorionic somatomammo-tropin hormone 2 (CSH2) mRNA
encoding Q7KZ35
CAGGGAGAGAGAACTGGCCAGGGTATAAAAAGGGCCCACAAGAGACCGGC
TCTAGGATCCCAAGGCCCAACTCCCCGAACCACTCAGGGTCCTGTGGACA
GCTCACCTAGCGGCAATGGCTGCAGGCTCCCGGACGTCCCTGCTCCTGGC
TTTTGCCCTGCTCTGCCTGCCCTGGCTTCAAGAGGCTGGTGCCGTCCAAA
CCGTTCCGTTATCCAGGCTTTTTGACCACGCTATGCTCCAAGCCCATCGC
GCGCACCAGCTGGCCATTGACACCTACCAGGAGTTTAGGCTGGAAGACGG
CAGCCGCCGGACTGGGCAGATCCTCAAGCAGACCTACAGCAAGTTTGACA
CAAACTCACACAACCATGACGCACTGCTCAAGAACTACGGGCTGCTCTAC
TGCTTCAGGAAGGACATGGACAAGGTCGAGACATTCCTGCGCATGGTGCA
GTGCCGCTCTGTAGAGGGTAGCTGTGGCTTCTAGGTGCCCGCGTGGCATC
CTGTGACCGACCCCTCCCCAGTGCCTCTCCTGGCCCTGGAAGGTGCCACT
CCAGTGCCCATCAGCCTTGTCCTAATAAAATTAAGTTGTATCATTTCA
e) CSHL1 primer
>gi|12545375|ref|NM_022579.1| Homo sapiens
chorionic somatomammo-tropin hormone-like 1
(CSHL1), transcript variant 1, mRNA encoding
Q14406-1
AGCATCCCAAGGCCCGACTCCCCGCACCACTCAGGGTCCTGTGGACAGCT
CACCTAGCGGCAATGGCTGCAGGCTCCCGGACGTCCCTGCTCCTGGCTTT
TGCCCTGCTCTGCCTGCCCTGGCTTCAAGAGGCTGGTGCCGTCCAAACCG
TTCCCTTATCCAGGCTTTTTAAAGAGGCTATGCTCCAAGCCCATCGCGCA
CACCAGCTGGCCATTGACACCTACCAGGAGTTTATAAGCTCTTGGGGAAT
GGAAGCCTATATCACAAAGGAACAGAAGTATTCATTCCTGCATGACTCCC
AGACCTCCTTCTGCTTCTCAGACTCTATTCCGACATCCTCCAACATGGAG
GAAACGCAGCAGAAATCCAACTTAGAGCTGCTCCACATCTCCCTGCTGCT
CATCGAGTCGCGGCTGGAGCCCGTGCGGTTCCTCAGGAGTACCTTCACCA
ACAACCTGGTGTATGACACCTCGGACAGCGATGACTATCACCTCCTAAAG
GACCTAGAGGAAGGCATCCAAATGCTGATGGGGAGGCTGGAAGACGGCAG
CCACCTGACTGGGCAGACCCTCAAGCAGACCTACAGCAAGTTTGACACAA
ACTCGCACAACCATGACGCACTGCTCAAGAACTACGGGCTGCTCCACTGC
TTCAGGAAGGACATGGACAAGGTCGAGACATTCCTGCGCATGGTGCAGTG
CCGCTCTGTGGAGGGCAGCTGTGGCTTCTAGGGGCCCGCGTGGCATCCTG
TGACCCCTCCCCAGTGCCTCTCCTGGCCCTGAAGGTGCCACTCCAGTGCC
CACCAGCCTTGTCTTAATAAAATTAAGTTGTATTGTT
>gi|12545380|ref|NM_022581.1| Homo sapiens
chorionic somatomammo-tropin hormone-like 1
(CSHL1), transcript variant 2, mRNA encoding
Q14406-2
AGCATCCCAAGGCCCGACTCCCCGCACCACTCAGGGTCCTGTGGACAGCT
CACCTAGCGGCAATGGCTGCAGGCTCCCGGACGTCCCTGCTCCTGGCTTT
TGCCCTGCTCTGCCTGCCCTGGCTTCAAGAGGCTGGTGCCGTCCAAACCG
TTCCCTTATCCAGGCTTTTTAAAGAGGCTATGCTCCAAGCCCATCGCGCA
CACCAGCTGGCCATTGACACCTACCAGGAGTTTATAAGCTCTTGGGGAAT
GGACTCTATTCCGACATCCTCCAACATGGAGGAAACGCAGCAGAAATCCA
ACTTAGAGCTGCTCCACATCTCCCTGCTGCTCATCGAGTCGCGGCTGGAG
CCCGTGCGGTTCCTCAGGAGTACCTTCACCAACAACCTGGTGTATGACAC
CTCGGACAGCGATGACTATCACCTCCTAAAGGACCTAGAGGAAGGCATCC
AAATGCTGATGGGGAGGCTGGAAGACGGCAGCCACCTGACTGGGCAGACC
CTCAAGCAGACCTACAGCAAGTTTGACACAAACTCGCACAACCATGACGC
ACTGCTCAAGAACTACGGGCTGCTCCACTGCTTCAGGAAGGACATGGACA
AGGTCGAGACATTCCTGCGCATGGTGCAGTGCCGCTCTGTGGAGGGCAGC
TGTGGCTTCTAGGGGCCCGCGTGGCATCCTGTGACCCCTCCCCAGTGCCT
CTCCTGGCCCTGAAGGTGCCACTCCAGTGCCCACCAGCCTTGTCTTAATA
AAATTAAGTTGTATTGTT

The derived PCR primers for the amplification of the growth hormone tDNAs are shown in the following table 2. These PCR primers are only examples.

Further suitable PCR primers for the detection of transgenic DNA which encodes the growth hormone, could be, in relation to the shown example, shortened or extended, or shifted towards the 5′- or 3′-ends, respectively, as long as such primers are located within the highly conserved transition area of two adjacent exons.

TABLE 2
Examples of growth hormone PCR primers.

| | | | | GC |
|---|---|---|---|---|
| SEQ | | | Tm | content |
| ID No. | Primer name | Primer sequence | [° C.] | [%] |
| 1 | GH1s | CAATGGCTACAGGCTCCCG | 61.2 | 63.2 |
| 2 | GH2-CSHL1-CSH2s | GCAATGGCTGCAGGCTCC | 62.0 | 66.7 |
| 3 | CSH1s | GCAATGGCTCCAGGCTCC | 60.9 | 66.7 |
| 4 | GH1as | AGCAGCTCTAGGTTGGATTTCTG | 59.9 | 47.8 |
| 5 | GH2as | GAGCAGCTCTAGGTTAGATTTCTGC | 60.7 | 48.0 |
| 6 | CSHL1as | GAGCAGCTCTAAGTTGGATTTCTG | 59.8 | 45.8 |
| 7 | CSH1-CSH2as1 | GAGCAGCTCTAGATTGGATTTCTG | 59.7 | 45.8 |
| 8 | CSH1-CSH2as2 | CTTCCAGCCTAAACTCCTGGTAG | 59.6 | 52.2 |

,,s“ means sense primer, ,,as“ means antisense primer; GH1 means growth hormone 1, GH2 means growth hormone 2, CSH1 means chorionic somatomammo-tropin hormone 1, CSH2 means chorionic somatomammo-tropin hormone 2, CSHL1 means chorionic somatomammo-tropin hormone-like 1.

### 3.1.1 Pre-PCR

For all growth hormone genes the pre-PCR is designed as a multiplex PCR. For this a mixture is used comprising the following primers which in each case are used at 0.1 μM: GH1s (sense primer), GH2-CSHL1-CSH2s (sense primer), CSH1s (sense primer), GH1as (antisense primer), GH2 as (antisense primer), CSHL1as (antisense primer), CSH1-CSH2 as1 (antisense primer), CSH1-CSH2 as (antisense primer). In the pre-PCR the PCR amplificate GH-Pre is obtained.

### 3.1.2 Secondary PCR

The secondary PCR is performed in a gene-specific manner.

a) for GH1:

The gene-specific secondary PCR is performed with the primer pair GH1s (sense primer) and GH1as1 (antisense prime), each of which is used at a concentration of 0.3 μM. The GH1 PCR amplificate has a length of 307 bp for P01241 and P01241-3±4, and 262 bp for P01241-2.

### b) for GH2

The gene-specific secondary PCR is performed with GH2-CSHL1-CSH2s (sense primer) and GH2 as (antisense primer), each of which is used at a concentration of 0.3 μM. The GH2 PCR product for the variants 1 to 3 has a length of 309 base pairs and for the variant 4 a length of 264 base pairs.

### c) for CSH1

In the gene-specific secondary PCR for the amplification of the coding sequence for the protein P01243 (CSH1-I-PCR amplificate) the following primers are used, each of which are used at a concentration of 0.3 μM: CSH1s (sense primer) and CSH1-CSH2as1 (antisense primer). The resulting amplificate has a length of 309 base pairs.

For the amplification of the coding sequence for the protein Q7KZ35 (CSH1-II-PCR amplificate) the following primer pairs are used, each of which is used at a concentration of 0.3 μM: CSH1s (sense primer) and CSH1-CSH2 as2 (antisense primer). The CSH1-II PCR amplificate has a length of 184 base pairs.

### d) for CSH2

In the gene-specific secondary PCR for the amplification of the coding sequence for the protein P01243 (CSH2-I-PCR amplificate) the following primers are used, each of which are used at a concentration of 0.3 μM: GH2-CSHL1-CSH2s (sense primer) and CSH1-CSH2 as1 (antisense primer). The resulting amplificate has a length of 309 base pairs.

For the amplification of the coding sequence for the protein Q7KZ35 (CSH2-II-PCR amplificate) the following primer pairs are used, each of which is used at a concentration of 0.3 μM: GH2-CSHL1-CSH2s (sense primer) and CSH1-CSH2 as2 (antisense primer). The CSH2-II PCR amplificate has a length of 184 base pairs.

### e) for CSHL1

The gene-specific secondary PCR is performed with G2-GSHL1-CSH2s (sense primer) and CSHL1as (antisense primer). The CSHL1 PCR amplificate for the protein Q14406-1 has a length of 324 base pairs and for Q14406-1 has a length of 255 base pairs.

3.2 Primers for the Detection of Gene Therapy or Doping with tDNA Encoding Erythropoietin

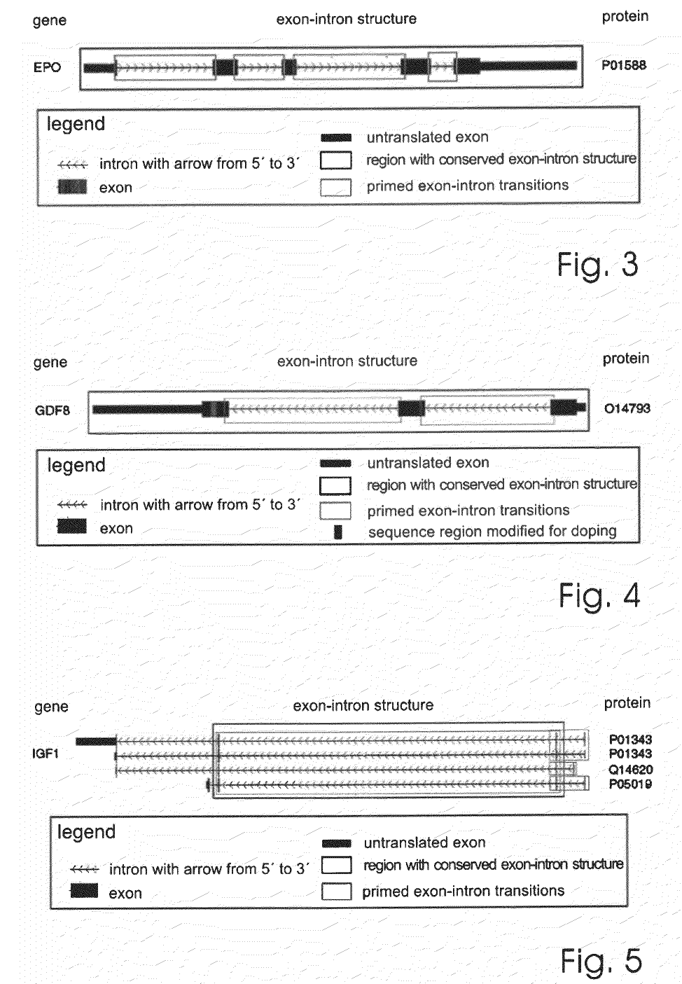

FIG. 3 shows the structure of the exons and introns of the reference mRNA sequence for the only known protein variant of erythropoietin (EPO). Therefore, the complete mRNA reference sequence is in principle suitable for the construction of intron-spanning primers. The light boxes show the areas by the way of example, which can be used for the construction of primers.

In the following for the genomic DNA sequence for EPO, it is exemplarily shown how the PCR primers for the amplification of EPO tDNA can be designed. The intron sequences are dark grey in color, the coding sequence (cds) for the gene therapy- or doping-relevant protein is black in color, the sense primer is bold, the antisense primer is underlined and not bold, and the segments which could be sense as well as antisense primers are bold and underlined.¹ stands for the beginning or the end of primer 1,² stands for the beginning or the end of primer 2.

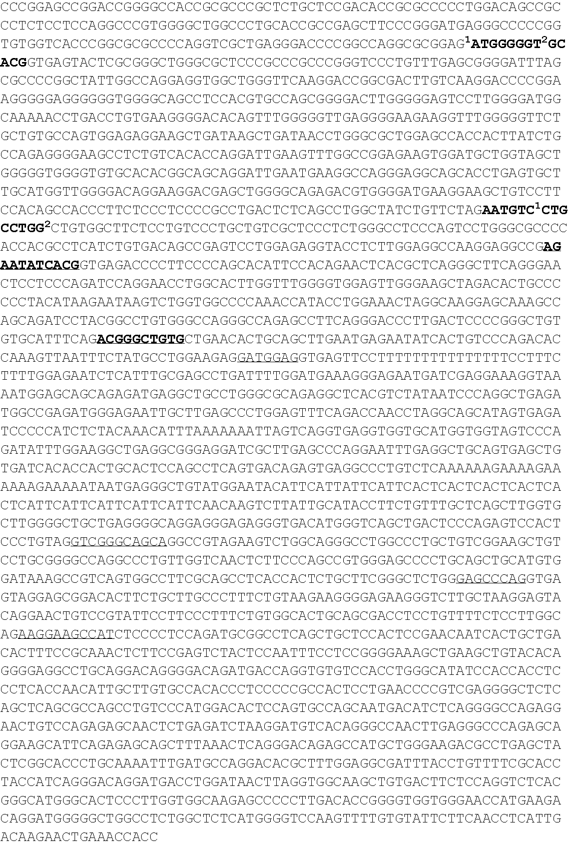

In the following the corresponding EPO mRNA is shown.

>gi|62240996|ref|NM_000799.2| Homo sapiens
erythropoietin (EPO), mRNA
CCCGGAGCCGGACCGGGGCCACCGCGCCCGCTCTGCTCCGACACCGCGCC
CCCTGGACAGCCGCCCTCTCCTCCAGGCCCGTGGGGCTGGCCCTGCACCG
CCGAGCTTCCCGGGATGAGGGCCCCCGGTGTGGTCACCCGGCGCGCCCCA
GGTCGCTGAGGGACCCCGGCCAGGCGCGGAG¹ATGGGGGT²GCACGAATG
TC¹b CTGCC²TGGCTGTGGCTTCTCCTGTCCCTGCTGTCGCTCCCTCTGGG
CCTCCCAGTCCTGGGCGCCCCACCACGCCTCATCTGTGACAGCCGAGTCC
TGGAGAGGTACCTCTTGGAGGCCAAGGAGGCCGAGAATATCACGACGGGC
TGTGCTGAACACTGCAGCTTGAATGAGAATATCACTGTCCCAGACACCAA
AGTTAATTTCTATGCCTGGAAGAGGATGGAGGTCGGGCAGCAGGCCGTAG
AAGTCTGGCAGGGCCTGGCCCTGCTGTCGGAAGCTGTCCTGCGGGGCCAG
GCCCTGTTGGTCAACTCTTCCCAGCCGTGGGAGCCCCTGCAGCTGCATGT
GGATAAAGCCGTCAGTGGCCTTCGCAGCCTCACCACTCTGCTTCGGG²CT
CTGG¹GAGCCCAGAAGGA²AGCCAT¹CTCCCCTCCAGATGCGGCCTCAGC
TGCTCCACTCCGAACAATCACTGCTGACACTTTCCGCAAACTCTTCCGAG
TCTACTCCAATTTCCTCCGGGGAAAGCTGAAGCTGTACACAGGGGAGGCC
TGCAGGACAGGGGACAGATGACCAGGTGTGTCCACCTGGGCATATCCACC
ACCTCCCTCACCAACATTGCTTGTGCCACACCCTCCCCCGCCACTCCTGA
ACCCCGTCGAGGGGCTCTCAGCTCAGCGCCAGCCTGTCCCATGGACACTC
CAGTGCCAGCAATGACATCTCAGGGGCCAGAGGAACTGTCCAGAGAGCAA
CTCTGAGATCTAAGGATGTCACAGGGCCAACTTGAGGGCCCAGAGCAGGA
AGCATTCAGAGAGCAGCTTTAAACTCAGGGACAGAGCCATGCTGGGAAGA
CGCCTGAGCTCACTCGGCACCCTGCAAAATTTGATGCCAGGACACGCTTT
GGAGGCGATTTACCTGTTTTCGCACCTACCATCAGGGACAGGATGACCTG
GAGAACTTAGGTGGCAAGCTGTGACTTCTCCAGGTCTCACGGGCATGGGC
ACTCCCTTGGTGGCAAGAGCCCCCTTGACACCGGGGTGGTGGGAACCATG
AAGACAGGATGGGGGCTGGCCTCTGGCTCTCATGGGGTCCAAGTTTTGTG
TATTCTTCAACCTCATTGACAAGAACTGAAACCACCAAAAAAAAAAAA

The derived PCR primers for the amplification of the EPO tDNA are shown in the following Table 3. Also these PCR primers are only examples.

TABLE 3
Examples for erythropoietin primer.

| SEQ | | | Tm | GC content |
|---|---|---|---|---|
| ID No. | Primer name | Primer sequence | [° C.] | [%] |
| 9 | EPOs1 | ATGGGGGTGCACGAATGTC | 60.8 | 57.9 |
| 10 | EPOas3 | ATGGCTTCCTTCTGGGCTC | 58.9 | 57.9 |
| 11 | EPOs1 − 11 | GGTGCACGAATGTCCTGCC | 61.6 | 63.3 |
| 12 | EPOas1 | CACAGCCCGTCGTGATATTCT | 60.0 | 52.4 |
| 13 | EPOs2 + 3 | AGAATATCACGACGGGCTGTG | 60.0 | 52.4 |
| 14 | EPOas2 | TGCTGCCCGACCTCCATC | 62.1 | 66.7 |
| 15 | EPOs2 + 3 | AGAATATCACGACGGGCTGTG | 60.0 | 52.4 |
| 16 | EPOas3 − II | TCCTTCTGGGCTCCCAGAG | 59.7 | 63.2 |
| 17 | EPOs1 − II | GGTGCACGAATGTCCTGCC | 61.6 | 63.3 |
| 18 | EPOas3 − II | TCCTTCTGGGCTCCCAGAG | 59.7 | 62.2 |

,,s“ means Sense primer, ,,as“ means antisense primer; EPO means erythropoietin.

3.2.1 Pre-PCR

The PCR amplificate EPO 1-3, obtainable with the primer pair EPOs1 (sense primer) and EPOas3 (antisense primer), each of which is used at a concentration of 0.3 μM, has a length of 437 bp.

### 3.2.2 Secondary PCR

The PCR product EPO1, obtainable with the primer pair EPOs1-II (sense primer) and EPOas1 (antisense primer), has a length of 169 base pairs, the PCR product EPO2, obtainable with the primer pair EPOs2+3 (sense primer) and EPOas2 (antisense primer), has a length of 109 base pairs, the PCR product EP03, obtainable with the primer pair EPOs2+3 (sense primer) and EPOas3-II (antisense primer), has a length of 289 base pairs, and the PCR product EPO1-3-II, obtainable with the primer pairs EPOs1-II (sense primer) and EPOas3-2 (antisense primer), has a length of 423 base pairs. Each of the primers for the secondary PCR is used at a concentration of 0.3 μM.

3.3 Primer for the Detection of Gene Therapy or Doping with a tDNA Encoding the Myostatin Inhibitor

FIG. 4 shows the structure of the exons and introns of the reference mRNA sequence for the gene therapy- or doping-relevant protein regions of myostatin (GDF8). In principle the complete mRNA reference sequence is suitable for the construction of intron-spanning primers. The light boxes show exemplarily the regions which were used for the construction of primers.

In the selection of the primers it has been taken care that in exon 3 a dark gray accentuated sequence area is located which is modified or deleted with the object of a performance enhancement. This results in a dominant negative myostatin inhibitor (GDF8 inhibitor) which is not able to inhibit the muscle growth like the natural myostatin (GDF8). This area is left out in the construction of the primers.

In the following, on the basis of the genomic DNA sequence for myostatin it is exemplarily shown how the PCR Primers for the amplification of myostatin inhibitor tDNA can be designed. The intron sequences are dark grey in color, the coding sequence (cds) for the doping-relevant protein is black in color, the sense primer is bold and the antisense primer is underlined and not bold.¹ stands for the beginning or the end of primer 1,² stands for the beginning or the end of primer 2.

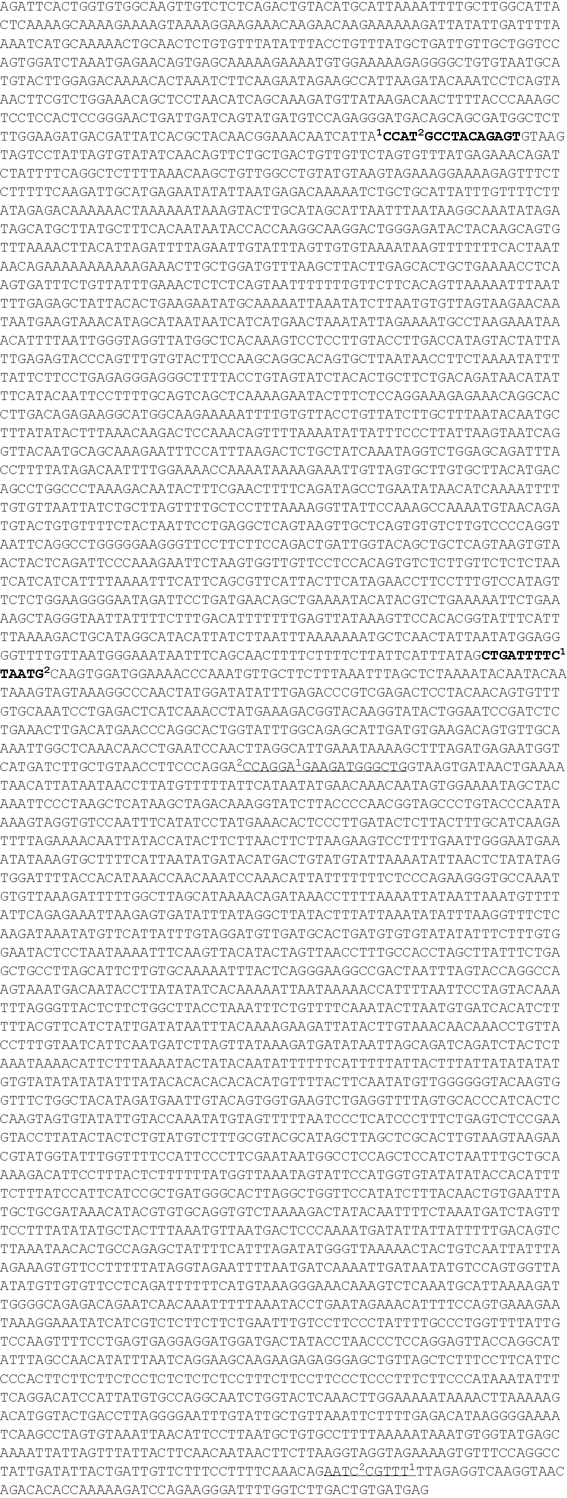

Beginning of the sequence part which is modified in an efficient gene therapy or doping:

CACTCAACAGAATCACGATGCTGTCGTTACCCTCTAACTGTGGATTTTGA
AGCTTTTGGATGGGATTGGATTATCGCTCCTAAAAGATATAAGGCCAATT
ACTGCTCTGGAGAGTGTGAATTTGTATTTTTACAAAAATATCCTCATACT
CATCTGGTACACCAAGCAAACCCCAGAGGTTCAGCAGGCCCTTGCTGTAC
TCCCACAAAGATGTCTCCAATTAATATGCTATATTTTAATGGCAAAGAAC
AAATAATATATGGGAAAATTCCAGCGATGGTAGTAGACCGCTGTGGGTGC
TCATGAGATTTATATTAAGCGTTCATAACTTCCTAAAACATGGAAGGTTT
TCCCCTCAACAATTTTGAAGCTGTGAAATTAAGTACCACAGGCTATAGGC
CTAGAGTATGCTACAGTCACTTAAGCATAAGCTACAGTATGTAAACTAAA
AGGGGGAATATATGCAATGGTTGGCATTTAACCATCCAAACAAATCATAC
AAGAAAGTTTTATGATTTCCAGAGTTTTTGAGCTAGAAGGAGATCAAATT
ACATTTATGTTCCTATATATTACAACATCGGCGAGGAAATGAAAGCGATT
CTCCTTGAGTTCTGATGAATTAAAGGAGTATGCTTTAAAGTCTATTTCTT
TAAAGTTTTGTTTAATATTTACAGAAAAATCCACATACAGTATTGGTAAA
ATGCAGGATTGTTATATACCATCATTCGAATCATCCTTAAACACTTGAAT
TTATATTGTATGGTAGTATACTTGGTAAGATAAAATTCCACAAAAATAGG
GATGGTGCAGCATATGCAATTTCCATTCCTATTATAATTGACACAGTACA
TTAACAATCCATGCCAACGGTGCTAATACGATAGGCTGAATGTCTGAGGC
TACCAGGTTTATCACATAAAAAACATTCAGTAAAATAGTAAGTTTCTCTT
TTCTTCAGGTGCATTTTCCTACACCTCCAAATGAGGAATGGATTTTCTTT
AATGTAAGAAGAATCATTTTTCTAGAGGTTGGCTTTCAATTCTGTAGCAT
ACTTGGAGAAACTGCATTATCTTAAAAGGCAGTCAAATGGTGTTTGTTTT
TATCAAAATGTCAAAATAACATACTTGGAGAAGTATGTAATTTTGTCTTT
GGAAAATTACAACACTGCCTTTGCAACACTGCAGTTTTTATGGTAAAATA
ATAGAAATGATCGACTCTATCAATATTGTATAAAAAGACTGAAACAATGC
ATTTATATAATATGTATACAATATTGTTTTGTAAATAAGTGTCTCCTTTT
TTATTTACTTTGGTATATTTTTACACTAAGGACATTTCAAATTAAGTACT
AAGGCACAAAGACATGTCATGCATCACAGAAAAGCAACTACTTATATTTC
AGAGCAAATTAGCAGATTAAATAGTGGTCTTAAAACTCCATATGTTAATG
ATTAGATGGTTATATTACAATCATTTTATATTTTTTTACATGATTAACAT
TCACTTATGGATTCATGATGGCTGTATAAAGTGAATTTGAAATTTCAATG
GTTTACTGTCATTGTGTTTAAATCTCAACGTTCCATTATTTTAATACTTG
CAAAAACATTACTAAGTATACCAAAATAATTGACTCTATTATCTGAAATG
AAGAATAAACTGATGCTATCTCAACAATAACTGTTACTTTTATTTTATAA
TTTGATAATGAATATATTTCTGCATTTATTTACTTCTGTTTTGTAAATTG
GGATTTTGTTAATCAAATTTATTGTACTATGACTAAATGAAATTATTTCT
TACATCTAATTTGTAGAAACAGTATAAGTTATATTAAAGTGTTTTCACAT
TTTTTTGAAAGAC

In the following, the corresponding mRNA encoding myostatin is shown:

>gi|4885258|ref|NM_005259.1| Homo sapiens
myostatin (GDF8), mRNA
AGATTCACTGGTGTGGCAAGTTGTCTCTCAGACTGTACATGCATTAAAAT
TTTGCTTGGCATTACTCAAAAGCAAAAGAAAAGTAAAAGGAAGAAACAAG
AACAAGAAAAAAGATTATATTGATTTTAAAATCATGCAAAAACTGCAACT
CTGTGTTTATATTTACCTGTTTATGCTGATTGTTGCTGGTCCAGTGGATC
TAAATGAGAACAGTGAGCAAAAAGAAAATGTGGAAAAAGAGGGGCTGTGT
AATGCATGTACTTGGAGACAAAACACTAAATCTTCAAGAATAGAAGCCAT
TAAGATACAAATCCTCAGTAAACTTCGTCTGGAAACAGCTCCTAACATCA
GCAAAGATGTTATAAGACAACTTTTACCCAAAGCTCCTCCACTCCGGGAA
CTGATTGATCAGTATGATGTCCAGAGGGATGACAGCAGCGATGGCTCTTT
GGAAGATGACGATTATCACGCTACAACGGAAACAATCATTA¹CCAT²GCC
TACAGAGTCTGATTTTC¹TAATGC²AAGTGGATGGAAAACCCAAATGTTG
CTTCTTTAAATTTAGCTCTAAAATACAATGTTTGTGCAAATCCTGAGACT
CATCAAACCTATGAAAGACGGTACAAGGTATACTGGAATCCGATCTCTGA
AACTTGACATGAACCCAGGCACTGGTATTTGGCAGAGCATTGATGTGAAG
ACAGTGTTGCAAAATTGGCTCAAACAACCTGAATCCAACTTAGGCATTGA
AATAAAAGCTTTAGATGAGAATGGTCATGATCTTGCTGTAACCTTCCCAG
AA²CCAG¹GAGAAGATGGGCTGAATC²CGTTT¹TTAGAGGTCAAGGTAAC
AGACACACCAAAAAGATCCAGAAGGGATTTTGGTCTTGACTGTGATGAG

The beginning of the sequence part which is modified for an efficient gene therapy or doping purposes:

CACTCAACAGAATCACGATGCTGTCGTTACCCTCTAACTGTGGATTTTGA
AGCTTTTGGATGGGATTGGATTATCGCTCCTAAAAGATATAAGGCCAATT
ACTGCTCTGGAGAGTGTGAATTTGTATTTTTACAAAAATATCCTCATACT
CATCTGGTACACCAAGCAAACCCCAGAGGTTCAGCAGGCCCTTGCTGTAC
TCCCACAAAGATGTCTCCAATTAATATGCTATATTTTAATGGCAAAGAAC
AAATAATATATGGGAAAATTCCAGCGATGGTAGTAGACCGCTGTGGGTGC
TCATGAGATTTATATTAAGCGTTCATAACTTCCTAAAACATGGAAGGTTT
TCCCCTCAACAATTTTGAAGCTGTGAAATTAAGTACCACAGGCTATAGGC
CTAGAGTATGCTACAGTCACTTAAGCATAAGCTACAGTATGTAAACTAAA
AGGGGGAATATATGCAATGGTTGGCATTTAACCATCCAAACAAATCATAC
AAGAAAGTTTTATGATTTCCAGAGTTTTTGAGCTAGAAGGAGATCAAATT
ACATTTATGTTCCTATATATTACAACATCGGCGAGGAAATGAAAGCGATT
CTCCTTGAGTTCTGATGAATTAAAGGAGTATGCTTTAAAGTCTATTTCTT
TAAAGTTTTGTTTAATATTTACAGAAAAATCCACATACAGTATTGGTAAA
ATGCAGGATTGTTATATACCATCATTCGAATCATCCTTAAACACTTGAAT
TTATATTGTATGGTAGTATACTTGGTAAGATAAAATTCCACAAAAATAGG
GATGGTGCAGCATATGCAATTTCCATTCCTATTATAATTGACACAGTACA
TTAACAATCCATGCCAACGGTGCTAATACGATAGGCTGAATGTCTGAGGC
TACCAGGTTTATCACATAAAAAACATTCAGTAAAATAGTAAGTTTCTCTT
TTCTTCAGGGGCATTTTCCTACACCTCCAAATGAGGAATGGATTTTCTTT
AATGTAAGAAGAATCATTTTTCTAGAGGTTGGCTTTCAATTCTGTAGCAT
ACTTGGAGAAACTGCATTATCTTAAAAGGCAGTCAAATGGTGTTTGTTTT
TATCAAAATGTCAAAATAACATACTTGGAGAAGTATGTAATTTTGTCTTT
GGAAAATTACAACACTGCCTTTGCAACACTGCAGTTTTTATGGTAAAATA
ATAGAAATGATCGACTCTATCAATATTGTATAAAAAGACTGAAACAATGC
ATTTATATAATATGTATACAATATTGTTTTGTAAATAAGTGTCTCCTTTT
TTATTTACTTTGGTATATTTTTACACTAAGGACATTTCAAATTAAGTACT
AAGGCACAAAGACATGTCATGCATCACAGAAAAGCAACTACTTATATTTC
AGAGCAAATTAGCAGATTAAATAGTGGTCTTAAAACTCCATATGTTAATG
ATTAGATGGTTATATTACAATCATTTTATATTTTTTTACATGATTAACAT
TCACTTATGGATTCATGATGGCTGTATAAAGTGAATTTGAAATTTCAATG
GTTTACTGTCATTGTGTTTAAATCTCAACGTTCCATTATTTTAATACTTG
CAAAAACATTACTAAGTATACCAAAATAATTGACTCTATTATCTGAAATG
AAGAATAAACTGATGCTATCTCAACAATAACTGTTACTTTTATTTTATAA
TTTGATAATGAATATATTTCTGCATTTATTTACTTCTGTTTTGTAAATTG
GGATTTTGTTAATCAAATTTATTGTACTATGACTAAATGAAATTATTTCT
TACATCTAATTTGTAGAAACAGTATAAGTTATATTAAAGTGTTTTCACAT
TTTTTTGAAAGACAAAAA

The derived exemplary PCR primers for the amplification of the myostatin inhibitor tDNA are summarized in the following Table 4.

TABLE 4
examples for GDF8 primer.

| SEQ | | | Tm | GC content |
|---|---|---|---|---|
| ID No. | Primer name | Primer sequence | [° C.] | [%] |
| 19 | GDF8s1 | CCATGCCTACAGAGTCTGATTTTC | 60.0 | 45.8 |
| 20 | GDF8as1 | AAACGGATTCAGCCCATCTTC | 60.7 | 47.6 |
| 21 | GDF8s2 | GCCTACAGAGTCTGATTTTCTAATGC | 60.6 | 42.3 |
| 22 | GDF8as2 | GATTCAGCCCATCTTCTCCTGG | 62.5 | 54.5 |

,,s“ means sense primer, ,,as“ means antisense primer; GDF8 means myostatin (,,growth factor differentiation factor“)/myostatin inhibitor.

### 3.3.1 Pre-PCR

The PCR product GDF8-1, which is obtainable by the use of the primers GDF8s1 (sense primer) and GDF8 as1 (antisense primer), each of which is used at a concentration of 0.3 μM, has a length of 398 bp.

### 3.3.2 Secondary PCR

The PCR product GDF8-2, which is obtainable by the use of the primers GDFs2 (sense primer) and GDF8 as2 (antisense primer), each of which is used at a concentration of 0.3 μM, has a length of 389 bp.

3.4 Primers for the Detection of Gene Therapy or Doping with IGF1 tDNA

FIG. 5 shows the structure of the exons and introns of the four reference mRNA sequences for the known doping-relevant protein variants of IGF1. Only the exon-intron transition of exon 2 and 3 is completely conserved. The light boxes show exemplarily which region was used for the construction of primers. The variant which encodes the protein Q1462 is the only variant which does not comprise any conservation between exon 1 and 2 and does therefore not require an individual primer.
(a) Primers for the PCR for the variants M11568, M29644 and NM_{—}000618 (P01343 and P05019):

The genomic DNA sequence of IGF1 is shown in the region of the mRNA>chr12:101314008-101376808 (reverse complement). The intron sequences are dark grey in color, the coding sequence (cds) for the gene therapy- or doping-relevant protein is black in color, the sense primer is bold and the antisense primer is underlined and not bold.¹ stands for the beginning or the end of primer 1,² stands for the beginning or the end of primer 2.

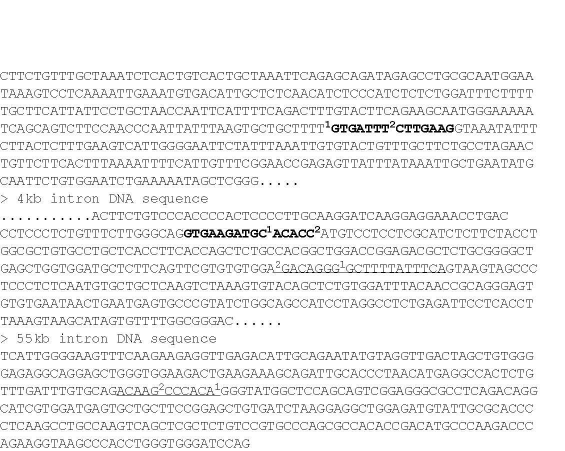

(b) Primers for the PCR for the variant M37484

The genomic DNA of IGF1 is shown in the region of the mRNA>chr12:101315008-101376808 (reverse complement). The intron sequences are dark grey in color, the coding sequence (cds) for the gene therapy- or doping-relevant protein is black in color, the sense primer is bold and the antisense primer is underlined and not bold.¹ stands for the beginning or the end of primer 1,² stands for the beginning or the end of primer 2.

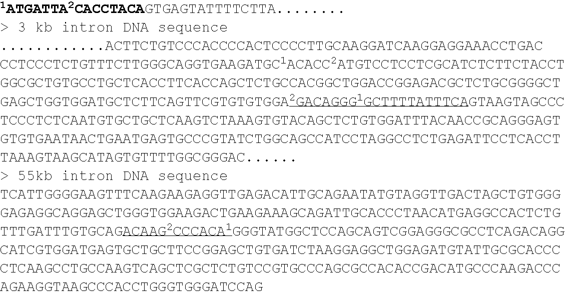

The corresponding mRNAs for the different IGF1 variants are as follows:

>gi|19923111|ref|NM_000618.2| Homo sapiens
insulin-like growth factor 1 (somatomedin C)
(IGF1), mRNA for protein P01343
TCACTGTCACTGCTAAATTCAGAGCAGATTAGAGCCTGCGCAATGGAATA
AAGTCCTCAAAATTGAAATGTGACATTGCTCTCAACATCTCCCATCTCTC
TGGATTTCCTTTTGCTTCATTATTCCTGCTAACCAATTCATTTTCAGACT
TTGTACTTCAGAAGCAATGGGAAAAATCAGCAGTCTTCCAACCCAATTAT
TTAAGTGCTGCTTTT¹GTGATTT²CTTGAAGGTGAAGATGC¹ACACC²AT
GTCCTCCTCGCATCTCTTCTACCTGGCGCTGTGCCTGCTCACCTTCACCA
GCTCTGCCACGGCTGGACCGGAGACGCTCTGCGGGGCTGAGCTGGTGGAT
GCTCTTCAGTTCGTGTGTGGA²GTACAGGG¹GCTTTTATTTCAACAAG²C
CCACA¹GGGTATGGCTCCAGCAGTCGGAGGGCGCCTCAGACAGGCATCGT
GGATGAGTGCTGCTTCCGGAGCTGTGATCTAAGGAGGCTGGAGATGTATT
GCGCACCCCTCAAGCCTGCCAAGTCAGCTCGCTCTGTCCGTGCCCAGCGC
CACACCGACATGCCCAAGACCCAGAAGGAAGTACATTTGAAGAACGCAAG
TAGAGGGAGTGCAGGAAACAAGAACTACAGGATGTAGGAAGACCCTCCTG
AGGAGTGAAGAGTGACATGCCACCGCAGGATCCTTTGCTCTGCACGAGTT
ACCTGTTAAACTTTGGAACACCTACCAAAAAATAAGTTTGATAACATTTA
AAAGATGGGCGTTTCCCCCAATGAAATACACAAGTAAACATTCCAACATT
GTCTTTAGGAGTGATTTGCACCTTGCAAAAATGGTCCTGGAGTTGGTAGA
TTGCTGTTGATCTTTTATCAATAATGTTCTATAGAAAAGAAAAAAAAATA
TATATATATATATATCTTAGTCCCTGCCTCTCAAGAGCCACAAATGCATG
GGTGTTGTATAGATCCAGTTGCACTAAATTCCTCTCTGAATCTTGGCTGC
TGGAGCCATTCATTCAGCAACCTTGTCTAAGTGGTTTATGAATTGTTTCC
TTATTTGCACTTCTTTCTACACAACTCGGGCTGTTTGTTTTACAGTGTCT
GATAATCTTGTTAGTCTATACCCACCACCTCCCTTCATAACCTTTATATT
TGCCGAATTTGGCCTCCTCAAAAGCAGCAGCAAGTCGTCAAGAAGCACAC
CAATTCTAACCCACAAGATTCCATCTGTGGCATTTGTACCAAATATAAGT
TGGATGCATTTTATTTTAGACACAAAGCTTTATTTTTCCACATCATGCTT
ACAAAAAAGAATAATGCAAATAGTTGCAACTTTGAGGCCAATCATTTTTA
GGCATATGTTTTAAACATAGAAAGTTTCTTCAACTCAAAAGAGTTCCTTC
AAATGATGAGTTAATGTGCAACCTAATTAGTAACTTTCCTCTTTTTATTT
TTTCCATATAGAGCACTATGTAAATTTAGCATATCAATTATACAGGATAT
ATCAAACAGTATGTAAAACTCTGTTTTTTAGTATAATGGTGCTATTTTGT
AGTTTGTTATATGAAAGAGTCTGGCCAAAACGGTAATACGTGAAAGCAAA
ACAATAGGGGAAGCCTGGAGCCAAAGATGACACAAGGGGAAGGGTACTGA
AAACACCATCCATTTGGGAAAGAAGGCAAAGTCCCCCCAGTTATGCCTTC
CAAGAGGAACTTCAGACACAAAAGTCCACTGATGCAAATTGGACTGGCGA
GTCCAGAGAGGAAACTGTGGAATGGAAAAAGCAGAAGGCTAGGAATTTTA
GCAGTCCTGGTTTCTTTTTCTCATGGAAGAAATGAACATCTGCCAGCTGT
GTCATGGACTCACCACTGTGTGACCTTGGGCAAGTCACTTCACCTCTCTG
TGCCTCAGTTTCCTCATCTGCAAAATGGGGGCAATATGTCATCTACCTAC
CTCAAAGGGGTGGTATAAGGTTTAAAAAGATAAAGATTCAGATTTTTTTA
CCCTGGGTTGCTGTAAGGGTGCAACATCAGGGCGCTTGAGTTGCTGAGAT
GCAAGGAATTCTATAAATAACCCATTCATAGCATAGCTAGAGATTGGTGA
ATTGAATGCTCCTGACATCTCAGTTCTTGTCAGTGAAGCTATCCAAATAA
CTGGCCAACTAGTTGTTAAAAGCTAACAGCTCAATCTCTTAAAACACTTT
TCAAAATATGTGGGAAGCATTTGATTTTCAATTTGATTTTGAATTCTGCA
TTTGGTTTTATGAATACAAAGATAAGTGAAAAGAGAGAAAGGAAAAGAAA
AAGGAGAAAAACAAAGAGATTTCTACCAGTGAAAGGGGAATTAATTACTC
TTTGTTAGCACTCACTGACTCTTCTATGCAGTTACTACATATCTAGTAAA
ACCTTGTTTAATACTATAAATAATATTCTATTCATTTTGAAAAACACAAT
GATTCCTTCTTTTCTAGGCAATATAAGGAAAGTGATCCAAAATTTGAAAT
ATTAAAATAATATCTAATAAAAAGTCACAAAGTTATCTTCTTTAACAAAC
TTTACTCTTATTCTTAGCTGTATATACATTTTTTTAAAAAGTTTGTTAAA
ATATGCTTGACTAGAGTTTCAGTTGAAAGGCAAAAACTTCCATCACAACA
AGAAATTTCCCATGCCTGCTCAGAAGGGTAGCCCCTAGCTCTCTGTGAAT
GTGTTTTATCCATTCAACTGAAAATTGGTATCAAGAAAGTCCACTGGTTA
GTGTACTAGTCCATCATAGCCTAGAAAATGATCCCTATCTGCAGATCAAG
ATTTTCTCATTAGAACAATGAATTATCCAGCATTCAGATCTTTCTAGTCA
CCTTAGAACTTTTTGGTTAAAAGTACCCAGGCTTGATTATTTCATGCAAA
TTCTATATTTTACATTCTTGGAAAGTCTATATGAAAAACAAAAATAACAT
CTTCAGTTTTTCTCCCACTGGGTCACCTCAAGGATCAGAGGCCAGGAAAA
AAAAAAAAGACTCCCTGGATCTCTGAATATATGCAAAAAGAAGGCCCCAT
TTAGTGGAGCCAGCAATCCTGTTCAGTCAACAAGTATTTTAACTCTCAGT
CCAACATTATTTGAATTGAGCACCTCAAGCATGCTTAGCAATGTTCTAAT
CACTATGGACAGATGTAAAAGAAACTATACATCATTTTTGCCCTCTGCCT
GTTTTCCAGACATACAGGTTCTGTGGAATAAGATACTGGACTCCTCTTCC
CAAGATGGCACTTCTTTTTATTTCTTGTCCCCAGTGTGTACCTTTTAAAA
TTATTCCCTCTCAACAAAACTTTATAGGCAGTCTTCTGCAGACTTAACAT
GTTTTCTGTCATAGTTAGATGTGATAATTCTAAGAGTGTCTATGACTTAT
TTCCTTCACTTAATTCTATCCACAGTCAAAAATCCCCCAAGGAGGAAAGC
TGAAAGATGCAACTGCCAATATTATCTTTCTTAACTTTTTCCAACACATA
ATCCTCTCCAACTGGATTATAAATAAATTGAAAATAACTCATTATACCAA
TTCACTATTTTATTTTTTAATGAATTAAAACTAGAAAACAAATTGATGCA
AACCCTGGAAGTCAGTTGATTACTATATACTACAGCAGAATGACTCAGAT
TTCATAGAAAGGAGCAACCAAAATGTCACAACCAAAACTTTACAAGCTTT
GCTTCAGAATTAGATTGCTTTATAATTCTTGAATGAGGCAATTTCAAGAT
ATTTGTAAAAGAACAGTAAACATTGGTAAGAATGAGCTTTCAACTCATAG
GCTTATTTCCAATTTAATTGACCATACTGGATACTTAGGTCAAATTTCTG
TTCTCTCTTGCCCAAATAATATTAAAGTATTATTTGAACTTTTTAAGATG
AGGCAGTTCCCCTGAAAAAGTTAATGCAGCTCTCCATCAGAATCCACTCT
TCTAGGGATATGAAAATCTCTTAACACCCACCCTACATACACAGACACAC
ACACACACACACACACACACACACACACACACACATTCACCCTAAGGATC
CAATGGAATACTGAAAAGAAATCACTTCCTTGAAAATTTTATTAAAAAAC
AAACAAACAAACAAAAAGCCTGTCCACCCTTGAGAATCCTTCCTCTCCTT
GGAACGTCAATGTTTGTGTAGATGAAACCATCTCATGCTCTGTGGCTCCA
GGGTTTCTGTTACTATTTTATGCACTTGGGAGAAGGCTTAGAATAAAAGA
TGTAGCACATTTTGCTTTCCCATTTATTGTTTGGCCAGCTATGCCAATGT
GGTGCTATTGTTTCTTTAAGAAAGTACTTGACTAAAAAAAAAAGAAAAAA
AGAAAAAAAAGAAAGCATAGACATATTTTTTTAAAGTATAAAAACAACAA
TTCTATAGATAGATGGCTTAATAAAATAGCATTAGGTCTATCTAGCCACC
ACCACCTTTCAACTTTTTATCACTCACAAGTAGTGTACTGTTCACCAAAT
TGTGAATTTGGGGGTGCAGGGGCAGGAGTTGGAAATTTTTTAAAGTTAGA
AGGCTCCATTGTTTTGTTGGCTCTCAAACTTAGCAAAATTAGCAATATAT
TATCCAATCTTCTGAACTTGATCAAGAGCATGGAGAATAAACGCGGGAAA
AAAGATCTTATAGGCAAATAGAAGAATTTAAAAGATAAGTAAGTTCCTTA
TTGATTTTTGTGCACTCTGCTCTAAAACAGATATTCAGCAAGTGGAGAAA
ATAAGAACAAAGAGAAAAAATACATAGATTTACCTGCAAAAAATAGCTTC
TGCCAAATCCCCCTTGGGTATTCTTTGGCATTTACTGGTTTATAGAAGAC
ATTCTCCCTTCACCCAGACATCTCAAAGAGCAGTAGCTCTCATGAAAAGC
AATCACTGATCTCATTTGGGAAATGTTGGAAAGTATTTCCTTATGAGATG
GGGGTTATCTACTGATAAAGAAAGAATTTATGAGAAATTGTTGAAAGAGA
TGGCTAACAATCTGTGAAGATTTTTTGTTTCTTGGTTTTGTTTTTTTTTT
TTTTTTTACTTTATACAGTCTTTATGAATTTCTTAATGTTCAAAATGACT
TGGTTCTTTTCTTCTTTTTTTTATATCAGAATGAGGAATAATAAGTTAAA
CCCACATAGACTCTTTAAAACTATAGGCTAGATAGAAATGTATGTTTGAC
TTGTTGAAGCTATAATCAGACTATTTAAAATGTTTTGCTATTTTTAATCT
TAAAAGATTGTGCTAATTTATTAGAGCAGAACCTGTTTGGCTCTCCTCAG
AAGAAAGAATCTTTCCATTCAAATCACATGGCTTTCCACCAATATTTTCA
AAAGATAAATCTGATTTATGCAATGGCATCATTTATTTTAAAACAGAAGA
ATTGTGAAAGTTTATGCCCCTCCCTTGCAAAGACCATAAAGTCCAGATCT
GGTAGGGGGGCAACAACAAAAGGAAAATGTTGTTGATTCTTGGTTTTGGA
TTTTGTTTTGTTTTCAATGCTAGTGTTTAATCCTGTAGTACATATTTGCT
TATTGCTATTTTAATATTTTATAAGACCTTCCTGTTAGGTATTAGAAAGT
GATACATAGATATCTTTTTTGTGTAATTTCTATTTAAAAAAGAGAGAAGA
CTGTCAGAAGCTTTAAGTGCATATGGTACAGGATAAAGATATCAATTTAA
ATAACCAATTCCTATCTGGAACAATGCTTTTGTTTTTTAAAGAAACCTCT
CACAGATAAGACAGAGGCCCAGGGGATTTTTGAAGCTGTCTTTATTCTGC
CCCCATCCCAACCCAGCCCTTATTATTTTAGTATCTGCCTCAGAATTTTA
TAGAGGGCTGACCAAGCTGAAACTCTAGAATTAAAGGAACCTCACTGAAA
ACATATATTTCACGTGTTCCCTCTCTTTTTTTTCCTTTTTGTGAGATGGG
GTCTCGCACTGTCCCCCAGGCTGGAGTGCAGTGGCATGATCTCGGCTCAC
TGCAACCTCCACCTCCTGGGTTTAAGCGATTCTCCTGCCTCAGCCTCCTG
AGTAGCTGGGATTACAGGCACCCACCACTATGCCCGGCTAATTTTTTGGA
TTTTTAATAGAGACGGGGTTTTACCATGTTGGCCAGGTTGGACTCAAACT
CCTGACCTTGTGATTTGCCCGCCTCAGCCTCCCAAATTGCTGGGATTACA
GGCATGAGCCACCACACCCTGCCCATGTGTTCCCTCTTAATGTATGATTA
CATGGATCTTAAACATGATCCTTCTCTCCTCATTCTTCAACTATCTTTGA
TGGGGTCTTTCAAGGGGAAAAAAATCCAAGCTTTTTTAAAGTAAAAAAAA
AAAAAGAGAGGACACAAAACCAAATGTTACTGCTCAACTGAAATATGAGT
TAAGATGGAGACAGAGTTTCTCCTAATAACCGGAGCTGAATTACCTTTCA
CTTTCAAAAACATGACCTTCCACAATCCTTAGAATCTGCCTTTTTTTATA
TTACTGAGGCCTAAAAGTAAACATTACTCATTTTATTTTGCCCAAAATGC
ACTGATGTAAAGTAGGAAAAATAAAAACAGAGCTCTAAAATCCCTTTCAA
GCCACCCATTGACCCCACTCACCAACTCATAGCAAAGTCACTTCTGTTAA
TCCCTTAATCTGATTTTGTTTGGATATTTATCTTGTACCCGCTGCTAAAC
ACACTGCAGGAGGGACTCTGAAACCTCAAGCTGTCTACTTACATCTTTTA
TCTGTGTCTGTGTATCATGAAAATGTCTATTCAAAATATCAAAACCTTTC
AAATATCACGCAGCTTATATTCAGTTTACATAAAGGCCCCAAATACCATG
TCAGATCTTTTTGGTAAAAGAGTTAATGAACTATGAGAATTGGGATTACA
TCATGTATTTTGCCTCATGTATTTTTATCACACTTATAGGCCAAGTGTGA
TAAATAAACTTACAGACACTGAATTAATTTCCCCTGCTACTTTGAAACCA
GAAAATAATGACTGGCCATTCGTTACATCTGTCTTAGTTGAAAAGCATAT
TTTTTATTAAATTAATTCTGATTGTATTTGAAATTATTATTCAATTCACT
TATGGCAGAGGAATATCAATCCTAATGACTTCTAAAAATGTAACTAATTG
AATCATTATCTTACATTTACTGTTTAATAAGCATATTTTGAAAATGTATG
GCTAGAGTGTCATAATAAAATGGTATATCTTTCTTTAGTAATTACAAAAA
AAAAAAAAAAAAAAAAAAA
>gi|183111|gb;M11568.1|HUMGFIB Human insulin-like
growth factor IB (IGF-IB) cDNA according to the
mRNA for protein P01343
CTTCTGTTTGCTAAATCTCACTGTCACTGCTAAATTCAGAGCAGATAGAG
CCTGCGCAATGGAATAAAGTCCTCAAAATTGAAATGTGACATTGCTCTCA
ACATCTCCCATCTCTCTGGATTTCCTTTTGCTTCATTATTCCTGCTAACC
AATTCATTTTCAGACTTTGTACTTCAGAAGCAATGGGAAAAATCAGCAGT
CTTCCAACCCAATTATTTAAGTGCTGCTTTT¹GTGATTT²CTTGAAGGTG
AAGATGC¹ACACC²ATGTCCTCCTCGCATCTCTTCTACCTGGCGCTGTGC
CTGCTCACCTTCACCAGCTCTGCCACGGCTGGACCGGAGACGCTCTGCGG
GGCTGAGCTGGTGGATGCTCTTCAGTTCGTGTGTGGA²GACAGGG¹GCTT
TTATTTCAACAAG²CCCACA¹GGGTATGGCTCCAGCAGTCGGAGGGCGCC
TCAGACAGGCATCGTGGATGAGTGCTGCTTCCGGAGCTGTGATCTAAGGA
GGCTGGAGATGTATTGCGCACCCCTCAAGCCTGCCAAGTCAGCTCGCTCT
GTCCGTGCCCAGCGCCACACCGACATGCCCAAGACCCAGAAGTATCAGCC
CCCATCTACCAACAAGAACACGAAGTCTCAGAGAAGGAAAGGTTGGCCAA
AGACACATCCAGGAGGGGAACAGAAGGAGGGGACAGAAGCAAGTCTGCAG
ATCAGAGGAAAGAAGAAAGAGCAGAGGAGGGAGATTGGAAGTAGAAATGC
TGAATGCAGAGGCAAAAAAGGAAAATGAAGGACAGGAGGATTAAACAGAC
AGAGGCAAGGATGATGAGAGAGGAGCAGACAGCAAGAATGAAAAGCAGAA
AATACAATAGAGGAAATGAAGAAAAGTAGGCCTGCTGGAGCTAGATGATG
ATGTGATGGAAATAGAAGTAACCTTTTAGAGAATCTCGCTAAGAAACATG
GAGAAAACGGAAAAGAAAAATGTAATGCCCTAGAAAGCGCAAAGAAAGAC
AGTGGCAAAAATGAAAAAAAAAAAATAAAAATTATAAAAGAGGCAAAAAA
AGACACACTATTCTCTGCCCTCTAAAACACAATTAAATAAAAGAATTTAA
AT
>gi|184833.1: 151-564|M37484 Human insulin-like
growth factor I (IGF-I) mRNA, complete cds:
protein Q14620 (see FIG. 5)
1ATGATTA²CACCTACAGTGAAGATGC¹ACACC²ATGTCCTCCTCGCATC
TCTTCTACCTGGCGCTGTGCCTGCTCACCTTCACCAGCTCTGCCACGGCT
GGACCGGAGACGCTCTGCGGGGCTGAGCTGGTGGATGCTCTTCAGTTCGT
GTGTGGA²GACAGGG¹GCTTTTATTTCAACAAG²CCCACA¹GGGTATGGC
TCCAGCAGTCGGAGGGCGCCTCAGACAGGCATCGTGGATGAGTGCTGCTT
CCGGAGCTGTGATCTAAGGAGGCTGGAGATGTATTGCGCACCCCTCAAGC
CTGCCAAGTCAGCTCGCTCTGTCCGTGCCCAGCGCCACACCGACATGCCC
AAGACCCAGAAGGAAGTACATTTGAAGAACGCAAGTAGAGGGAGTGCAGG
AAACAAGAACTACAGGATGTAG
>gi|183111|gb;|11568.1|HUMGFIB human insulin-like
growth factor IB (IGF-IB) cDNA after mRNA for
protein P05019
CTTCTGTTTGCTAAATCTCACTGTCACTGCTAAATTCAGAGCAGATAGAG
CCTGCGCAATGGAATAAAGTCCTCAAAATTGAAATGTGACATTGCTCTCA
ACATCTCCCATCTCTCTGGATTTCCTTTTGCTTCATTATTCCTGCTAACC
AATTCATTTTCAGACTTTGTACTTCAGAAGCAATGGGAAAAATCAGCAGT
CTTCCAACCCAATTATTTAAGTGCTGCTTTT¹GTGATTT²CTTGAAGGTG
AAGATGC¹ACACC²ATGTCCTCCTCGCATCTCTTCTACCTGGCGCTGTGC
CTGCTCACCTTCACCAGCTCTGCCACGGCTGGACCGGAGACGCTCTGCGG
GGCTGAGCTGGTGGATGCTCTTCAGTTCGTGTGTGGA²GACAGGG¹GCTT
TTATTTCAACAAG²CCCACA¹GGGTATGGCTCCAGCAGTCGGAGGGCGCC
TCAGACAGGCATCGTGGATGAGTGCTGCTTCCGGAGCTGTGATCTAAGGA
GGCTGGAGATGTATTGCGCACCCCTCAAGCCTGCCAAGTCAGCTCGCTCT
GTCCGTGCCCAGCGCCACACCGACATGCCCAAGACCCAGAAGTATCAGCC
CCCATCTACCAACAAGAACACGAAGTCTCAGAGAAGGAAAGGTTGGCCAA
AGACACATCCAGGAGGGGAACAGAAGGAGGGGACAGAAGCAAGTCTGCAG
ATCAGAGGAAAGAAGAAAGAGCAGAGGAGGGAGATTGGAAGTAGAAATGC
TGAATGCAGAGGCAAAAAAGGAAAATGAAGGACAGGAGGATTAAACAGAC
AGAGGCAAGGATGATGAGAGAGGAGCAGACAGCAAGAATGAAAAGCAGAA
AATACAATAGAGGAAATGAAGAAAAGTAGGCCTGCTGGAGCTAGATGATG
ATGTGATGGAAATAGAAGTAACCTTTTAGAGAATCTCGCTAAGAAACATG
GAGAAAACGGAAAAGAAAAATGTAATGCCCTAGAAAGCGCAAAGAAAGAC
AGTGGCAAAAATGAAAAAAAAAAAATAAAAATTATAAAAGAGGCAAAAAA
AGACACACTATTCTCTGCCCTCTAAAACACAATTAAATAAAAGAATTTAA
AT

The derived exemplary PCR primers for the amplification of the IGF1 tDNA are summarized in the following Table 5.

TABLE 5
Examples for IGF1 primer.

| SEQ | | | Tm | GC content |
|---|---|---|---|---|
| ID No. | Primer name | Primer sequence | [° C.] | [%] |
| 23 | IGF1s1 | GTGATTTCTTGAAGGTGAAGATGC | 60.0 | 41.7 |
| 24 | IGF1as1 | TGTGGGCTTGTTGAAATAAAAGC | 61.4 | 39.1 |
| 25 | IGF1s1 − II | ATGATTACACCTACAGTGAAGATGC | 57.5 | 40.0 |
| 26 | IGF1s2 | CTTGAAGGTGAAGATGCACACC | 59.6 | 50.0 |
| 27 | IGF1as2 | CTTGTTGAAATAAAAGCCCCTGTC | 61.6 | 41.7 |
| 28 | IGF1s2 − II | CACCTACAGTGAAGATGCACACC | 59.6 | 52.2 |
| 29 | IGF1as2 | CTTGTTGAAATAAAAGCCCCTGTC | 61.6 | 41.7 |

,,s“ means sense primer, ,,as“ means antisense primer; IGF means insulin-like growth factor.

### 3.4.1 Pre-PCR

By the use of a multiplex PCR the PCR product IGF1-Pre is obtained, wherein the primer IGFs1 (sense primer) is used at a concentration of 0.3 μM, and IGF1as1 (antisense primer) and IGFs1-II (antisense primer) are each used at a concentration of 0.2 μM.

### 3.4.2 Secondary PCR

The PCR product IGF1-1 for the proteins P01343 and P05019, which is obtained by the use of the primers IGF1s2 (sense primer) and IGF1as2 (antisense primer), each of which is used at a concentration of 0.3 μM, has a length of 169 base pairs.

The PCR product IGF1-2 for the protein Q14620, which is obtained by the use of the primer pairs IGF1s2-II (sense primer) and ISF1as2 (antisense primer), each of which is used at a concentration of 0.3 μM, has a length of 170 bp.

3.5 Primers for the Detection of Gene Therapy or Doping with IGF2 tDNA

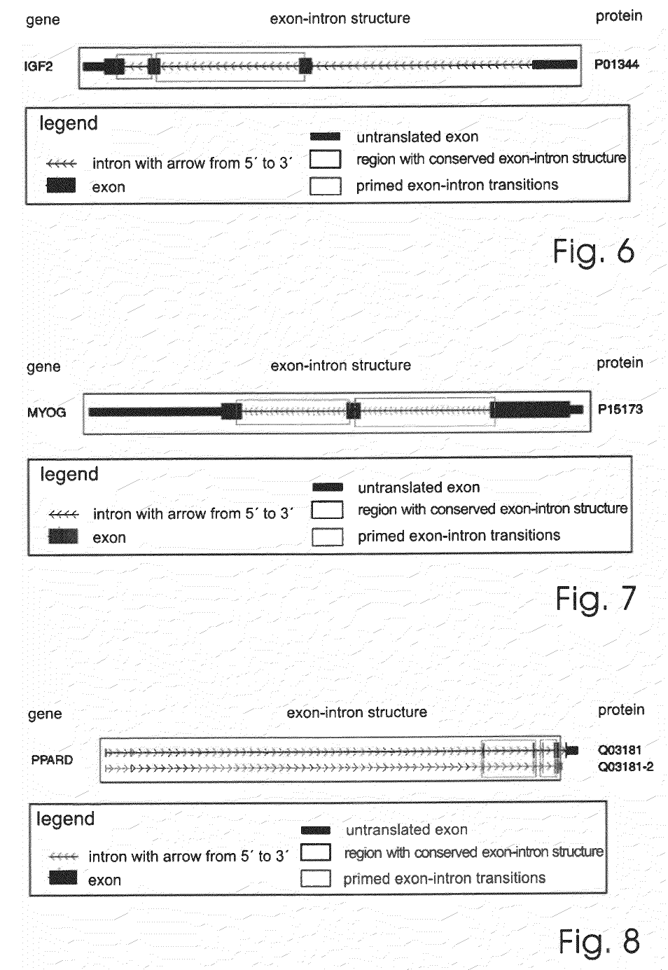

FIG. 6 shows the structure of the exons and introns of the reference mRNA sequence of IGF2. The complete mRNA sequence is, therefore, in principle suitable for the construction of intron-spanning primers. The light boxes show which parts were exemplarily used for the construction of primers.

The exon-intron transitions of the exons 2 to 4 are located within the protein encoding sequence (cds) and are, therefore, specially suited for the construction of intron-spanning primers. The exon 1 completely represents non-coding sequence and is, therefore, not relevant for an expression of the protein. For this reason this part of the sequence is left out in the selection of the primers.

The genomic DNA sequence of IGF2 in the region of the mRNA>chr11:2110105-2113505 (reverse complement) is shown. The intron sequences are dark grey in color, the coding sequence (cds) for the gene therapy- or doping-relevant protein is black in color, the sense primer is bold and the antisense primer is underlined and not bold.¹ stands for the beginning or the end of primer 1,² stands for the beginning or the end of primer 2.

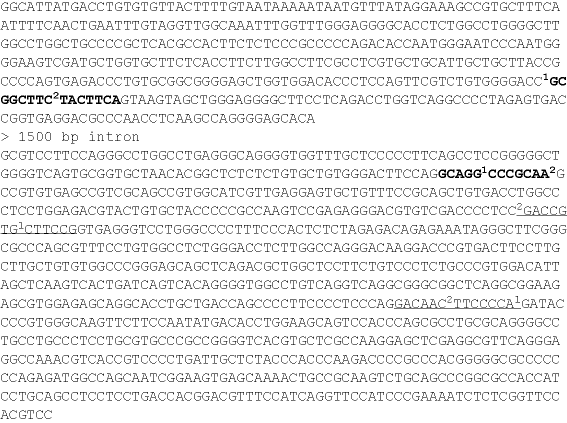

In the following the corresponding IGF2 mRNA is shown:

>gi|6453816|ref|NM_000612.2| Homo sapiens
insulin-like growth factor 2 (somatomedin A)
(IGF2), mRNA
TTCTCCCGCAACCTTCCCTTCGCTCCCTCCCGTCCCCCCCAGCTCCTAGC
CTCCGACTCCCTCCCCCCCTCACGCCCGCCCTCTCGCCTTCGCCGAACCA
AAGTGGATTAATTACACGCTTTCTGTTTCTCTCCGTGCTGTTCTCTCCCG
CTGTGCGCCTGCCCGCCTCTCGCTGTCCTCTCTCCCCCTCGCCCTCTCTT
CGGCCCCCCCCTTTCACGTTCACTCTGTCTCTCCCACTATCTCTGCCCCC
CTCTATCCTTGATACAACAGCTGACCTCATTTCCCGATACCTTTTCCCCC
CCGAAAAGTACAACATCTGGCCCGCCCCAGCCCGAAGACAGCCCGTCCTC
CCTGGACAATCAGACGAATTCTCCCCCCCCCCCCAAAAAAAAAAGCCATC
CCCCCGCTCTGCCCCGTCGCACATTCGGCCCCCGCGACTCGGCCAGAGCG
GCGCTGGCAGAGGAGTGTCCGGCAGGAGGGCCAACGCCCGCTGTTCGGTT
TGCGACACGCAGCAGGGAGGTGGGCGGCAGCGTCGCCGGCTTCCAGACAC
CAATGGGAATCCCAATGGGGAAGTCGATGCTGGTGCTTCTCACCTTCTTG
GCCTTCGCCTCGTGCTGCATTGCTGCTTACCGCCCCAGTGAGACCCTGTG
CGGCGGGGAGCTGGTGGACACCCTCCAGTTCGTCTGTGGGGACC¹GCGGC
TTC²TACTTCAGC¹AGGCCCGCAA²GCCGTGTGAGCCGTCGCAGCCGTGG
CATCGTTGAGGAGTGCTGTTTCCGCAGCTGTGACCTGGCCCTCCTGGAGA
CGTACTGTGCTACCCCCGCCAAGTCCGAGAGGGACGTGTCGACCCCTCC
²GACCGTG¹CTTCCGGACAAC²TTCCCCA¹GATACCCCGTGGGCAAGTTC
TTCCAATATGACACCTGGAAGCAGTCCACCCAGCGCCTGCGCAGGGGCCT
GCCTGCCCTCCTGCGTGCCCGCCGGGGTCACGTGCTCGCCAAGGAGCTCG
AGGCGTTCAGGGAGGCCAAACGTCACCGTCCCCTGATTGCTCTACCCACC
CAAGACCCCGCCCACGGGGGCGCCCCCCCAGAGATGGCCAGCAATCGGAA
GTGAGCAAAACTGCCGCAAGTCTGCAGCCCGGCGCCACCATCCTGCAGCC
TCCTCCTGACCACGGACGTTTCCATCAGGTTCCATCCCGAAAATCTCTCG
GTTCCACGTCCCCCTGGGGCTTCTCCTGACCCAGTCCCCGTGCCCCGCCT
CCCCGAAACAGGCTACTCTCCTCGGCCCCCTCCATCGGGCTGAGGAAGCA
CAGCAGCATCTTCAAACATGTACAAAATCGATTGGCTTTAAACACCCTTC
ACATACCCTCCCCCC

The derived exemplary PCR primers for the amplification of the IGF2 tDNA are summarized in the following Table 6.

TABLE 6
Examples for IGF2 primers.

| SEQ | | | | GC |
|---|---|---|---|---|
| ID | | | Tm | content |
| No. | Primer name | Primer sequence | [° C.] | [%] |
| 30 | IGF2s1 | GCGGCTTCTACTTCAGCAGG | 60.2 | 60.0 |
| 31 | IGF2as1 | TGGGGAAGTTGTCCGGAAG | 60.4 | 57.9 |
| 32 | IGF2s2 | TACTTCAGCAGGCCCGCAA | 62.4 | 57.9 |
| 33 | IGF2as2 | GTTGTCCGGAAGCACGGTC | 60.8 | 63.2 |

,,s“ means primer, ,,as“ means antisense primer: IGF means insulin-like growth factor.

### 3.5.1 Pre-PCR

The PCR product IGF1-1, which is obtained by the pre-PCR by the use of the primers IGF2s1 (sense primer) and IGF2 as 1 (antisense primer), each at a concentration of 0.3 μM, has a length of 177 bp.

### 3.5.2 Secondary PCR

The PCR product IGF1-2, which is obtained by the gene-specific secondary PCR by the use of the primers IGF2s2 (sense primer) and IGF2 as2 (antisense primer), each at a concentration of 0.3 μM, has a length of 162 bp.

3.6 Primers for the Detection of Gene Therapy or Doping with Myogenin tDNA

FIG. 7 shows the structure of the exons and introns of the reference mRNA sequence of the only known protein of myogenin (MYOG). Therefore, in principle, the complete mRNA reference sequence is suitable for the construction of intron-spanning primers. The light boxes indicate which parts were exemplarily used for the construction of primers.

The genomic DNA sequence of MYOG is shown in the region of the mRNA>chr1:201318883-201321789 (reverse complement). The intron sequences are dark grey in color, the coding sequence (cds) for the gene therapy- or doping-relevant protein is black in color, the sense primer is bold and the antisense primer is underlined and not bold.¹ stands for the beginning or the end of primer 1,² stands for the beginning or the end of primer 2.

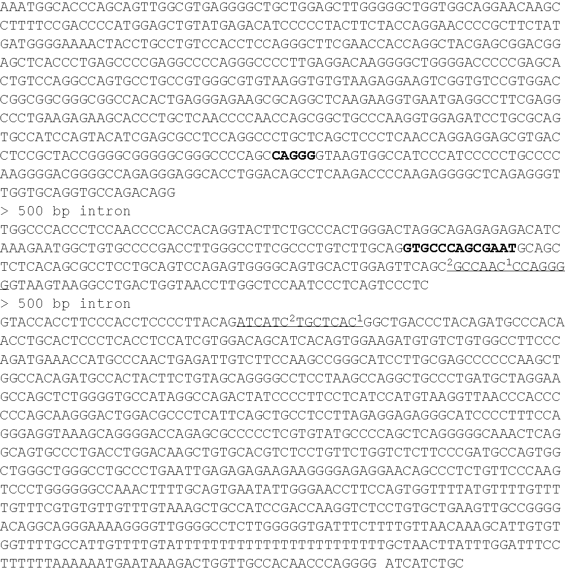

In the following the mRNA of myogenin is shown:

>gi|53729340|ref|NM_002479.3| Homo sapiens
myogenin (myogenic factor 4) (MYOG), mRNA
AAATGGCACCCAGCAGTTGGCGTGAGGGGCTGCTGGAGCTTGGGGGCTGG
TGGCAGGAACAAGCCTTTTCCGACCCCATGGAGCTGTATGAGACATCCCC
CTACTTCTACCAGGAACCCCGCTTCTATGATGGGGAAAACTACCTGCCTG
TCCACCTCCAGGGCTTCGAACCACCAGGCTACGAGCGGACGGAGCTCACC
CTGAGCCCCGAGGCCCCAGGGCCCCTTGAGGACAAGGGGCTGGGGACCCC
CGAGCACTGTCCAGGCCAGTGCCTGCCGTGGGCGTGTAAGGTGTGTAAGA
GGAAGTCGGTGTCCGTGGACCGGCGGCGGGCGGCCACACTGAGGGAGAAG
CGCAGGCTCAAGAAGGTGAATGAGGCCTTCGAGGCCCTGAAGAGAAGCAC
CCTGCTCAACCCCAACCAGCGGCTGCCCAAGGTGGAGATCCTGCGCAGTG
CCATCCAGTACATCGAGCGCCTCCAGGCCCTGCTCAGCTCCCTCAACCAG
GAGGAGCGTGACCTCCGCTACCGGGGCGGGGGCGGGCCCCAGCCAGGGGT
GCCCAGCGAATGCAGCTCTCACAGCGCCTCCTGCAGTCCAGAGTGGGGCA
GTGCACTGGAGTTCAGC²GCCAAC¹CCAGGGGATCATC²TGCTCAC¹GGC
TGACCCTACAGATGCCCACAACCTGCACTCCCTCACCTCCATCGTGGACA
GCATCACAGTGGAAGATGTGTCTGTGGCCTTCCCAGATGAAACCATGCCC
AACTGAGATTGTCTTCCAAGCCGGGCATCCTTGCGAGCCCCCCAAGCTGG
CCACAGATGCCACTACTTCTGTAGCAGGGGCCTCCTAAGCCAGGCTGCCC
TGATGCTAGGAAGCCAGCTCTGGGGTGCCATAGGCCAGACTATCCCCTTC
CTCATCCATGTAAGGTTAACCCACCCCCCAGCAAGGGACTGGACGCCCTC
ATTCAGCTGCCTCCTTAGAGGAGAGGGCATCCCCTTTCCAGGGAGGTAAA
GCAGGGGACCAGAGCGCCCCCTCGTGTATGCCCCAGCTCAGGGGGCAAAC
TCAGGAGCTTCCTTTTTATCATAACGCGGCCTCTAATTCCACCCCCCAAG
TGAAACGGTTTGAGAGACGCAGTGCCCTGACCTGGACAAGCTGTGCACGT
CTCCTGTTCTGGTCTCTTCCCGATGCCAGTGGCTGGGCTGGGCCTGCCCT
GAATTGAGAGAGAAGAAGGGGAGAGGAACAGCCCTCTGTTCCCAAGTCCC
TGGGGGGCCAAACTTTTGCAGTGAATATTGGGAACCTTCCAGTGGTTTTA
TGTTTTGTTTTGTTTCGTGTGTTGTTTGTAAAGCTGCCATCCGACCAAGG
TCTCCTGTGCTGAAGTTGCCGGGGACAGGCAGGGAAAAGGGGTTGGGGCC
TCTTGGGGGTGATTTCTTTTGTTAACAAAGCATCGTGTGGTTTTGCCATT
GTTTTGTATTTTTTTTTTTTTTTTTTTTTTTTGCTAACTTATTTGGATTT
CCTTTTTTAAAAAATGAATAAAGACTGGTTGCCA

The derived exemplary PCR primers for the amplification of the myogenin-tDNA are summarized in the following Table 7.

TABLE 7
Examples for MYOG primers.

| SEQ | | | | GC |
|---|---|---|---|---|
| ID | | | Tm | content |
| No. | Primer name | Primer sequence | [° C.] | [%] |
| 34 | MYOGs1 | CAGGGGTGCCCAGCGAAT | 63.7 | 66.7 |
| 35 | MYOGas1 | GTGAGCAGATGATCCCCTGG | 59.8 | 60.0 |
| 36 | MYOGas2 | GATGATCCCCTGGGTTGGC | 61.9 | 63.2 |

,,s“ means sense primer, ,,as“ means antisense primer; MYOG means myogenin.

### 3.6.1 Pre-PCR

The PCR product MYOG-1, which is obtained by the pre-PCR by the use of the primers MYOGs1 (sense primer) and MYOGas1 (antisense primer), each of which at a concentration of 0.3 μM, has a length of 100 bp.

3.6.2 The PCR Product MYOG-2, which is Obtained by the Gene-Specific Secondary PCR by the use of the primers MYOGs1 (sense primer) and MYOGas2 (antisense primer), each of which at a concentration of 0.3 μM, has a length of 93 bp.
3.7 Primers for the Detection of a Gene Therapy or Doping with tDNA Encoding Peroxisome Proliferator-Activated Receptor Delta

FIG. 8 shows the structure of the exons and introns of both of the reference mRNA sequences for the known protein variants of the peroxisome proliferator-activated receptor delta (PPARd). Only the exon-intron transitions between the exons 1 to 7 are conserved in both variants and can be used for the construction of intron-spanning primers. The light boxes show which areas were exemplarily used for the construction of primers.

Primers for the PCR for the variants Q03181 and Q03181-2:

The genomic DNA sequence of PPARd is shown in the region of the mRNA>chr6:35418313-35503933. The intron sequences and the non-translated regions are dark grey in color, the coding sequence (cds) of the doping-relevant protein is black in color, the cds which is not present in Q03181-2 is printed in italics, the sense primer is bold and the antisense primer is underlined and not bold.¹ stands for the beginning or the end of primer 1,² stands for the beginning or the end of primer 2.

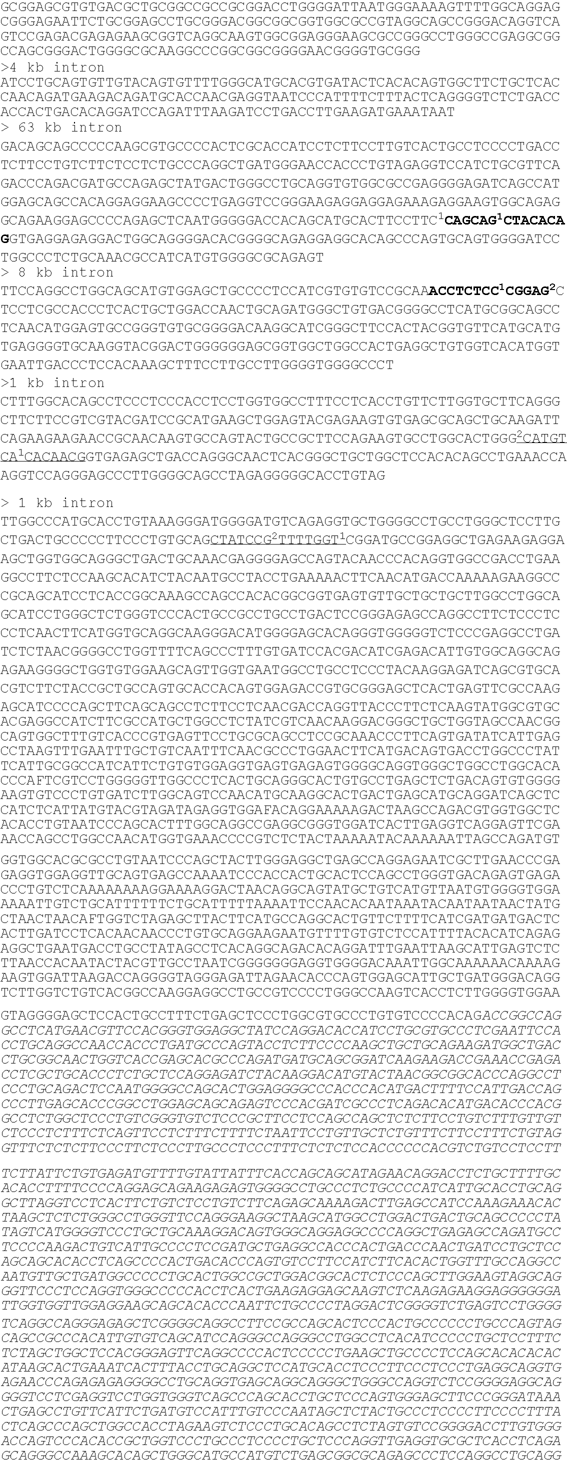

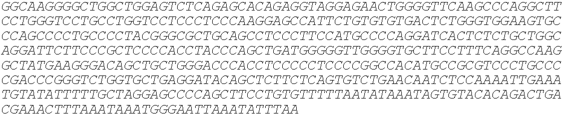

In the following the mRNAs for the variants of human PPARd are shown:

>gi|89886454|ref|NM_006238.3| Homo sapiens
peroxisome proliferator-activated receptor delta
(PPARd), mRNA for Q03181
GCGGAGCGTGTGACGCTGCGGCCGCCGCGGACCTGGGGATTAATGGGAAA
AGTTTTGGCAGGAGCGGGAGAATTCTGCGGAGCCTGCGGGACGGCGGCGG
TGGCGGCGTAGGCAGCCGGGACAGTGTTGTACAGTGTTTTGGGCATGCAC
GTGATACTCACACAGTGGCTTCTGCTCACCAACAGATGAAGACAGATGCA
CCAACGAGGCTGATGGGAACCACCCTGTAGAGGTCCATCTGCGTTCAGAC
CCAGACGATGCCAGAGCTATGACTGGGCCTGCAGGTGTGGCGCCGAGGGG
AGATCAGCCATGGAGCAGCCACAGGAGGAAGCCCCTGAGGTCCGGGAAGA
GGAGGAGAAAGAGGAAGTGGCAGAGGCAGAAGGAGCCCCAGAGCTCAATG
GGGGACCACAGCATGCACTTCCTTC¹CAGCAG²CTACACAGACCTCTCC
¹CGGAG²CTCCTCGCCACCCTCACTGCTGGACCAACTGCAGATGGGCTGT
GACGGGGCCTCATGCGGCAGCCTCAACATGGAGTGCCGGGTGTGCGGGGA
CAAGGCATCGGGCTTCCACTACGGTGTTCATGCATGTGAGGGGTGCAAGG
GCTTCTTCCGTCGTACGATCCGCATGAAGCTGGAGTACGAGAAGTGTGAG
CGCAGCTGCAAGATTCAGAAGAAGAACCGCAACAAGTGCCAGTACTGCCG
CTTCCAGAAGTGCCTGGCACTGGG²CATGTCA¹CACAACGCTATCCG²TT
TTGGT¹CGGATGCCGGAGGCTGAGAAGAGGAAGCTGGTGGCAGGGCTGAC
TGCAAACGAGGGGAGCCAGTACAACCCACAGGTGGCCGACCTGAAGGCCT
TCTCCAAGCACATCTACAATGCCTACCTGAAAAACTTCAACATGACCAAA
AAGAAGGCCCGCAGCATCCTCACCGGCAAAGCCAGCCACACGGCGCCCTT
TGTGATCCACGACATCGAGACATTGTGGCAGGCAGAGAAGGGGCTGGTGT
GGAAGCAGTTGGTGAATGGCCTGCCTCCCTACAAGGAGATCAGCGTGCAC
GTCTTCTACCGCTGCCAGTGCACCACAGTGGAGACCGTGCGGGAGCTCAC
TGAGTTCGCCAAGAGCATCCCCAGCTTCAGCAGCCTCTTCCTCAACGACC
AGGTTACCCTTCTCAAGTATGGCGTGCACGAGGCCATCTTCGCCATGCTG
GCCTCTATCGTCAACAAGGACGGGCTGCTGGTAGCCAACGGCAGTGGCTT
TGTCACCCGTGAGTTCCTGCGCAGCCTCCGCAAACCCTTCAGTGATATCA
TTGAGCCTAAGTTTGAATTTGCTGTCAAGTTCAACGCCCTGGAACTTGAT
GACAGTGACCTGGCCCTATTCATTGCGGCCATCATTCTGTGTGGAGACCG
GCCAGGCCTCATGAACGTTCCACGGGTGGAGGCTATCCAGGACACCATCC
TGCGTGCCCTCGAATTCCACCTGCAGGCCAACCACCCTGATGCCCAGTAC
CTCTTCCCCAAGCTGCTGCAGAAGATGGCTGACCTGCGGCAACTGGTCAC
CGAGCACGCCCAGATGATGCAGCGGATCAAGAAGACCGAAACCGAGACCT
CGCTGCACCCTCTGCTCCAGGAGATCTACAAGGACATGTACTAACGGCGG
CACCCAGGCCTCCCTGCAGACTCCAATGGGGCCAGCACTGGAGGGGCCCA
CCCACATGACTTTTCCATTGACCAGCCCTTGAGCACCCGGCCTGGAGCAG
CAGAGTCCCACGATCGCCCTCAGACACATGACACCCACGGCCTCTGGCTC
CCTGTGCCCTCTCTCCCGCTTCCTCCAGCCAGCTCTCTTCCTGTCTTTGT
TGTCTCCCTCTTTCTCAGTTCCTCTTTCTTTTCTAATTCCTGTTGCTCTG
TTTCTTCCTTTCTGTAGGTTTCTCTCTTCCCTTCTCCCTTGCCCTCCCTT
TCTCTCTCCACCCCCCACGTCTGTCCTCCTTTCTTATTCTGTGAGATGTT
TTGTATTATTTCACCAGCAGCATAGAACAGGACCTCTGCTTTTGCACACC
TTTTCCCCAGGAGCAGAAGAGAGTGGGGCCTGCCCTCTGCCCCATCATTG
CACCTGCAGGCTTAGGTCCTCACTTCTGTCTCCTGTCTTCAGAGCAAAAG
ACTTGAGCCATCCAAAGAAACACTAAGCTCTCTGGGCCTGGGTTCCAGGG
AAGGCTAAGCATGGCCTGGACTGACTGCAGCCCCCTATAGTCATGGGGTC
CCTGCTGCAAAGGACAGTGGGCAGGAGGCCCCAGGCTGAGAGCCAGATGC
CTCCCCAAGACTGTCATTGCCCCTCCGATGCTGAGGCCACCCACTGACCC
AACTGATCCTGCTCCAGCAGCACACCTCAGCCCCACTGACACCCAGTGTC
CTTCCATCTTCACACTGGTTTGCCAGGCCAATGTTGCTGATGGCCCCCTG
CACTGGCCGCTGGACGGCACTCTCCCAGCTTGGAAGTAGGCAGGGTTCCC
TCCAGGTGGGCCCCCACCTCACTGAAGAGGAGCAAGTCTCAAGAGAAGGA
GGGGGGATTGGTGGTTGGAGGAAGCAGCACACCCAATTCTGCCCCTAGGA
CTCGGGGTCTGAGTCCTGGGGTCAGGCCAGGGAGAGCTCGGGGCAGGCCT
TCCGCCAGCACTCCCACTGCCCCCCTGCCCAGTAGCAGCCGCCCACATTG
TGTCAGCATCCAGGGCCAGGGCCTGGCCTCACATCCCCCTGCTCCTTTCT
CTAGCTGGCTCCACGGGAGTTCAGGCCCCACTCCCCCTGAAGCTGCCCCT
CCAGCACACACACATAAGCACTGAAATCACTTTACCTGCAGGCTCCATGC
ACCTCCCTTCCCTCCCTGAGGCAGGTGAGAACCCAGAGAGAGGGGCCTGC
AGGTGAGCAGGCAGGGCTGGGCCAGGTCTCCGGGGAGGCAGGGGTCCTGC
AGGTCCTGGTGGGTCAGCCCAGCACCTGCTCCCAGTGGGAGCTTCCCGGG
ATAAACTGAGCCTGTTCATTCTGATGTCCATTTGTCCCAATAGCTCTACT
GCCCTCCCCTTCCCCTTTACTCAGCCCAGCTGGCCACCTAGAAGTCTCCC
TGCACAGCCTCTAGTGTCCGGGGACCTTGTGGGACCAGTCCCACACCGCT
GGTCCCTGCCCTCCCCTGCTCCCAGGTTGAGGTGCGCTCACCTCAGAGCA
GGGCCAAAGCACAGCTGGGCATGCCATGTCTGAGCGGCGCAGAGCCCTCC
AGGCCTGCAGGGGCAAGGGGCTGGCTGGAGTCTCAGAGCACAGAGGTAGG
AGAACTGGGGTTCAAGCCCAGGCTTCCTGGGTCCTGCCTGGTCCTCCCTC
CCAAGGAGCCATTCTGTGTGTGACTCTGGGTGGAAGTGCCCAGCCCCTGC
CCCTACGGGCGCTGCAGCCTCCCTTCCATGCCCCAGGATCACTCTCTGCT
GGCAGGATTCTTCCCGCTCCCCACCTACCCAGCTGATGGGGGTTGGGGTG
CTTCCTTTCAGGCCAAGGCTATGAAGGGACAGCTGCTGGGACCCACCTCC
CCCTCCCCGGCCACATGCCGCGTCCCTGCCCCGACCCGGGTCTGGTGCTG
AGGATACAGCTCTTCTCAGTGTCTGAACAATCTCCAAAATTGAAATGTAT
ATTTTTGCTAGGAGCCCCAGCTTCCTGTGTTTTTAATATAAATAGTGTAC
ACAGACTGACGAAACTTTAAATAAATGGGAATTAAATATTTAA
>gi|29171749|ref|NM_177435.1| Homo sapiens
peroxisome proliferator-activated receptor delta
(PPARd), transcript variant 2, mRNA for Q03181-2
GTTTTGGCAGGAGCGGGAGAATTCTGCGGAGCCTGCGGGACGGCGGCGGT
GGCGCCGTAGGCAGCCGGGACAGTGTTGTACAGTGTTTTGGGCATGCACG
TGATACTCACACAGTGGCTTCTGCTCACCAACAGATGAAGACAGATGCAC
CAACGAGGCTGATGGGAACCACCCTGTAGAGGTCCATCTGCGTTCAGACC
CAGACGATGCCAGAGCTATGACTGGGCCTGCAGGTGTGGCGCCGAGGGGA
GATCAGCCATGGAGCAGCCACAGGAGGAAGCCCCTGAGGTCCGGGAAGAG
GAGGAGAAAGAGGAAGTGGCAGAGGCAGAAGGAGCCCCAGAGCTCAATGG
GGGACCACAGCATGCACTTCCTTC¹CAGCAG²CTACACAGACCTCTCC¹C
GGAG²CTCCTCGCCACCCTCACTGCTGGACCAACTGCAGATGGGCTGTGA
CGGGGCCTCATGCGGCAGCCTCAACATGGAGTGCCGGGTGTGCGGGGACA
AGGCATCGGGCTTCCACTACGGTGTTCATGCATGTGAGGGGTGCAAGGGC
TTCTTCCGTCGTACGATCCGCATGAAGCTGGAGTACGAGAAGTGTGAGCG
CAGCTGCAAGATTCAGAAGAAGAACCGCAACAAGTGCCAGTACTGCCGCT
TCCAGAAGTGCCTGGCACTGGG²CATGTCA¹CACAACGCTATCCG²TTTT
GGT¹CGGATGCCGGAGGCTGAGAAGAGGAAGCTGGTGGCAGGGCTGACTG
CAAACGAGGGGAGCCAGTACAACCCACAGGTGGCCGACCTGAAGGCCTTC
TCCAAGCACATCTACAATGCCTACCTGAAAAACTTCAACATGACCAAAAA
GAAGGCCCGCAGCATCCTCACCGGCAAAGCCAGCCACACGGCGCCCTTTG
TGATCCACGACATCGAGACATTGTGGCAGGCAGAGAAGGGGCTGGTGTGG
AAGCAGTTGGTGAATGGCCTGCCTCCCTACAAGGAGATCAGCGTGCACGT
CTTCTACCGCTGCCAGTGCACCACAGTGGAGACCGTGCGGGAGCTCACTG
AGTTCGCCAAGAGCATCCCCAGCTTCAGCAGCCTCTTCCTCAACGACCAG
GTTACCCTTCTCAAGTATGGCGTGCACGAGGCCATCTTCGCCATGCTGGC
CTCTATCGTCAACAAGGACGGGCTGCTGGTAGCCAACGGCAGTGGCTTTG
TCACCCGTGAGTTCCTGCGCAGCCTCCGCAAACCCTTCAGTGATATCATT
GAGCCTAAGTTTGAATTTGCTGTCAAGTTCAACGCCCTGGAACTTGATGA
CAGTGACCTGGCCCTATTCATTGCGGCCATCATTCTGTGTGGAGGTGAGT
GAGAGTGGGGCAGGTGGGCTGGCCTGGCACACCCAGTCGTCCTGGGGGTT
GGCCCTCACTGCAGGGCACTGTGCCTGAGCTCTGACAGTGTGGGGAAGTG
TCCCTGTGATCTTGGCAGTGGAACATGCAAGGCACTGACTGAGCATGCAG
GATCAGCTCCATCTCATTATGTACGTAGATAGAGGTGGAGACAGGAAAAA
GACTAAGCCAGACGTGGTGGCTCACACCTGTAATCCCAGCACTTTGGCAG
GCCGAGGCGGGTGGATCACTTGAGGTCAGGAGTTCGAAACCAGCCTGGCC
AACATGGTGAAACCCCGTCTCTACTAAAAATACAAAAAATTAGCCAGATG
TGGTGGCACGCGCCTGTAATCCCAGCTACTTGGGAGGCTGAGCCAGGAGA
ATCGCTTGAACCCGAGAGGTGGAGGTTGCAGTGAGCCAAAATCCCACCAC
TGCACTCCAGCCTGGGTGACAGAGTGAGACCCTGTCTCAAAAAAAAGGAA
AAGGACTAACAGGCAGTATGCTGTCATGTTAATGTGGGGTGGAAAAATTG
TCTGCATTTTTTCTGCATTTTTAAAATTCCAACACAATAAATACAATAAT
AACTATGCTAAAAAAAAAAAAAAAAAAAAAAAAAA

The derived exemplary PCR primers for the amplification of the PPARd tDNA-tDNA are summarized in the following Table 8.

TABLE 8
Examples for PPARd primers.

| SEQ | | | Tm | GC content |
|---|---|---|---|---|
| ID No. | Primer name | Primer sequence | [° C.] | [%] |
| 37 | PPARDs1 | CAGCAGCTACACAGACCTCTCC | 59.1 | 59.1 |
| 38 | PPARDas1 | ACCAAAACGGATAGCGTTGTG | 60.3 | 57.6 |
| 39 | PPARDs2 | CTACACAGACCTCTCCCGGAG | 58.6 | 61.9 |
| 40 | PPARDas2 | CGGATAGCGTTGTGTGACATG | 59.7 | 52.4 |

,,s“ means sense primer, ,,as“ means antisense primer; PPARd means peroxisome proliferator-activated receptor delta.

### 3.7.1 Pre-PCR

The PCR product PPARD-1, which is obtained by the pre-PCR by the use of the primer PPARDs1 (sense primer) and PPARDas1 (antisense primer), each of which at a concentration of 0.3 μM, has a length of 322 bp.

### 3.7.2 Secondary PCR

The PCR product PPARD-2, which is obtainable for the proteins P01343 and P05019 by the gene-specific secondary PCR by the use of the primer pair PPARDs2 (sense primer) and PPARDas2 (antisense primer), each of which at a concentration of 0.3 μM, has a length of 309 bp.

3.8 Primers for the Detection of Gene Therapy or Doping with Calcineurin a Alpha tDNA

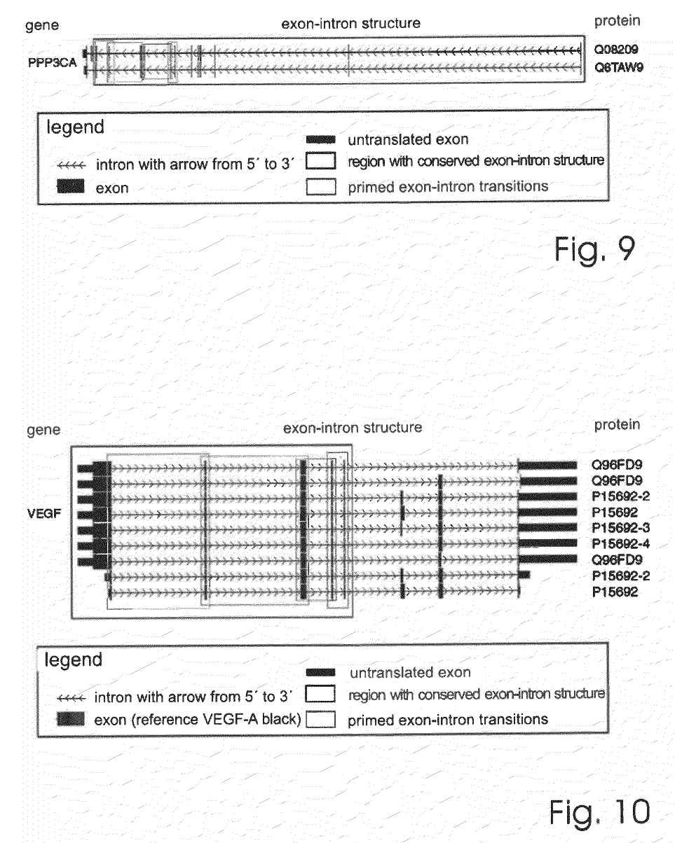

FIG. 9 shows the structure of the exons and introns of both of the reference mRNA sequences for the known protein variants of calcineurin A alpha (PPP3CA) which only differ from exon 13 onwards. The conserved exon-intron transitions between the exons 7 to 12 are used in the under-mentioned case to construct sense and antisense primers. In exon 14 the sequence region 1802 to 1868 bp in accordance to the reference sequence NM_{—}000944.2 is located, which is deleted or altered in the gene therapy or doping applications. This region is therefore missing in the construction of primers.

The genomic DNA sequence of PPP3CA is shown in the region of the mRNA>chr4:102301765-102625531 (reverse complement). The intron sequences, the coding sequence (cds) for the gene therapy- or doping-relevant protein is black in color, the sequence region 1802 to 1868 which is modified for doping purposes is printed in italics, the sense primer is bold and the antisense primer is underlined and not bold; sequences which can be used as sense but also as antisense primer are bold and underlined.¹ stands for the beginning or the end of primer 1,² stands for the beginning or the end of primer 2.

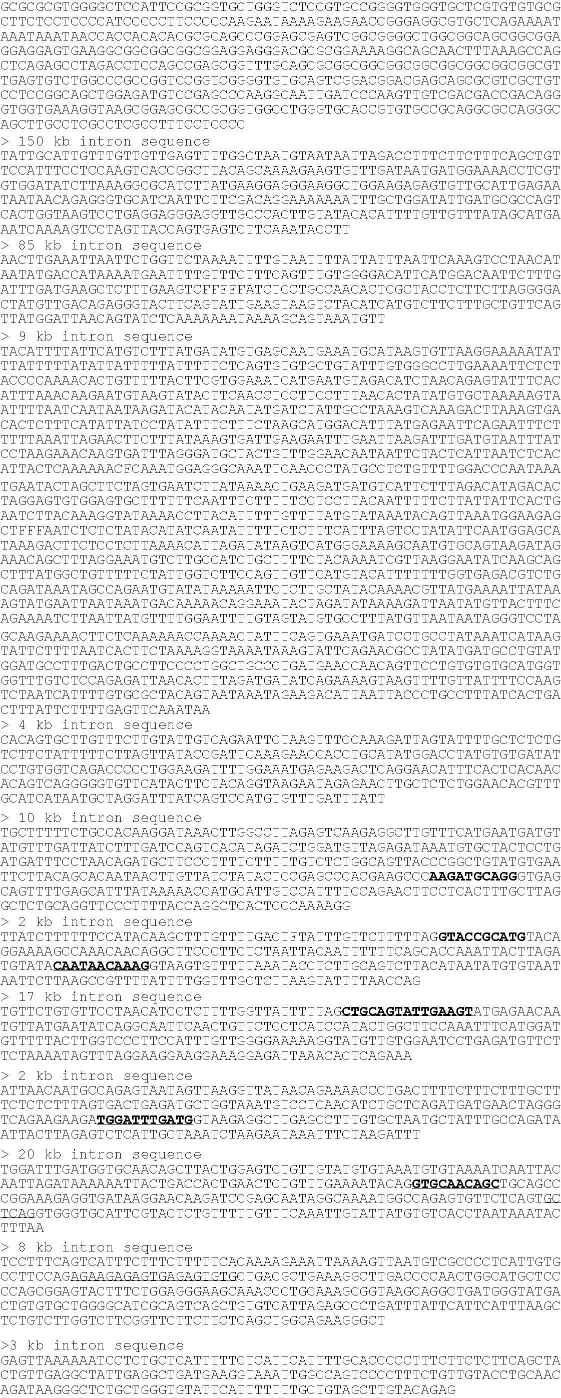

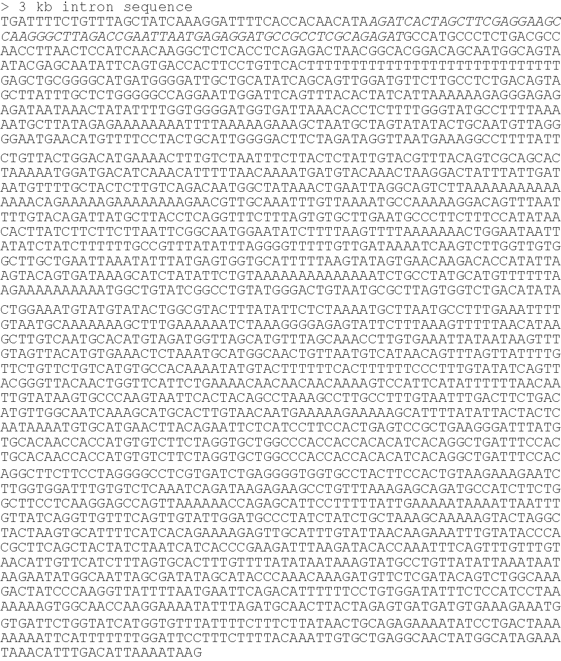

In the following the mRNA for PPP3CA is shown:

>gi|19923130|ref|NM_000944.2| Homo sapiens
protein phosphatase-3 (formerly 2B), catalytic
subunit, alpha isoform (calcineurin A alpha)
(PPP3CA), mRNA
GCGCGCGTGGGGCTCCATTCCGCGGTGCTGGGTCTCCGTGCCGGGGTGGG
TGCTCGTGTGTGCGCTTCTCCTCCCCATCCCCCTTCCCCCAAGAATAAAA
GAAGAACCGGGAGGCGTGCTCAGAAAATAAATAAATAACCACCACACACG
CGCAGCCCGGAGCGAGTCGGCGGGGCTGGCGGCAGCGGCGGAGGAGGAGT
GAAGGCGGCGGCGGCGGAGGAGGGACGCGCGGAAAAGGCAGCAACTTTAA
AGCCAGCTCAGAGCCTAGACCTCCAGCCGAGCGGTTTGCAGCGCGGCGGC
GGCGGCGGCGGCGGCGGCGGCGGCGTTGAGTGTCTGGCCCGCCGGTCCGG
TCGGGGTGTGCAGTCGGACGGACGAGCAGCGCGTCGCTGTCCTCCGGCAG
CTGGAGATGTCCGAGCCCAAGGCAATTGATCCCAAGTTGTCGACGACCGA
CAGGGTGGTGAAAGCTGTTCCATTTCCTCCAAGTCACCGGCTTACAGCAA
AAGAAGTGTTTGATAATGATGGAAAACCTCGTGTGGATATCTTAAAGGCG
CATCTTATGAAGGAGGGAAGGCTGGAAGAGAGTGTTGCATTGAGAATAAT
AACAGAGGGTGCATCAATTCTTCGACAGGAAAAAAATTTGCTGGATATTG
ATGCGCCAGTCACTGTTTGTGGGGACATTCATGGACAATTCTTTGATTTG
ATGAAGCTCTTTGAAGTCGGGGGATCTCCTGCCAACACTCGCTACCTCTT
CTTAGGGGACTATGTTGACAGAGGGTACTTCAGTATTGAATGTGTGCTGT
ATTTGTGGGCCTTGAAAATTCTCTACCCCAAAACACTGTTTTTACTTCGT
GGAAATCATGAATGTAGACATCTAACAGAGTATTTCACATTTAAACAAGA
ATGTAAAATAAAGTATTCAGAACGCGTATATGATGCCTGTATGGATGCCT
TTGACTGCCTTCCCCTGGCTGCCCTGATGAACCAACAGTTCCTGTGTGTG
CATGGTGGTTTGTCTCCAGAGATTAACACTTTAGATGATATCAGAAAATT
AGACCGATTCAAAGAACCACCTGCATATGGACCTATGTGTGATATCCTGT
GGTCAGACCCCCTGGAAGATTTTGGAAATGAGAAGACTCAGGAACATTTC
ACTCACAACACAGTCAGGGGGTGTTCATACTTCTACAGTTACCCGGCTGT
ATGTGAATTCTTACAGCACAATAACTTGTTATCTATACTCCGAGCCCACG
AAGCCCAAGATGCAGGGTACCGCATGTACAGGAAAAGCCAAACAACAGGC
TTCCCTTCTCTAATTACAATTTTTTCAGCACCAAATTACTTAGATGTATA
CAATAACAAAGCTGCAGTATTGAAGTATGAGAACAATGTTATGAATATCA
GGCAATTCAACTGTTCTCCTCATCCATACTGGCTTCCAAATTTCATGGAT
GTTTTTACTTGGTCCCTTCCATTTGTTGGGGAAAAAGTGACTGAGATGCT
GGTAAATGTCCTCAACATCTGCTCAGATGATGAACTAGGGTCAGAAGAAG
ATGGATTTGATGGTGCAACAGCTGCAGCCCGGAAAGAGGTGATAAGGAAC
AAGATCCGAGCAATAGGCAAAATGGCCAGAGTGTTCTCAGTGCTCAGAGA
AGAGAGTGAGAGTGTGCTGACGCTGAAAGGCTTGACCCCAACTGGCATGC
TCCCCAGCGGAGTACTTTCTGGAGGGAAGCAAACCCTGCAAAGCGCTACT
GTTGAGGCTATTGAGGCTGATGAAGCTATCAAAGGATTTTCACCACAACA
TAAGATCACTAGCTTCGAGGAAGCCAAGGGCTTAGACCGAATTAATGAGA
GGATGCCGCCTCGCAGAGATGCCATGCCCTCTGACGCCAACCTTAACTCC
ATCAACAAGGCTCTCACCTCAGAGACTAACGGCACGGACAGCAATGGCAG
TAATAGCAGCAATATTCAGTGACCACTTCCTGTTCACTTTTTTTTTTTTT
TTTTTTTTTTTTTGAGCTGCGGGGCATGATGGGGATTGCTGCATATCAGC
AGTTGGATGTTCTTGCCTCTGACAGTAGCTTATTTGCTCTGGGGGCCAGG
AATTGGATTCAGTTTACACTATCATTAAAAAAGAGGGAGAGAGATAATAA
ACTATATTTTGGTGGGGATGGTGATTAAACACCTCTTTTGGGTATGCCTT
TTAAAAATGCTTATAGAGAAAAAAATTTTAAAAAGAAAGCTAATGCTAGT
ATATACTGCAATGTTAGGGGAATGAACATGTTTTCCTACTGCATTGGGGA
CTTCTAGATAGGTTAATGAAAGGCCTTTTATTCTGTTACTGGACATGAAA
ACTTTGTCTAATTTCTTACTCTATTGTACGTTTACAGTCGCAGCACTAAA
AATGGATGACATCAAACATTTTTAACAAAATGATGTACAAACTAAGGACT
ATTTATTGATAATGTTTTGCTACTCTTGTCAGACAATGGCTATAAACTGA
ATTAGGCAGTCTTAAAAAAAAAAAAAAAACAGAAAAAGAAAAAAAAGAAC
GTTGCAAATTTGTTAAAATGCCAAAAAGGACAGTTTAATTTTGTACAGAT
TATGCTTACCTCAGGTTTCTTTAGTGTGCTTGAATGCCCTTCTTTCCATA
TAACACTTATCTTCTTCTTAATTCGGCAATGGAATATCTTTTAAGTTTTA
AAAAAACTGGAATAATTATATCTATCTTTTTTGCCGTTTATATTTAGGGG
TTTTTGTTGATAAAATCAAGTCTTGGTTGTGGCTTGCTGAATTAAATATT
TATGAGTGGTGCATTTTTAAGTATAGTGAACAAGACACCATATTAAGTAC
AGTGATAAAGCATCTATATTCTGTAAAAAAAAAAAAAATCTGCCTATGCA
TGTTTTTTAAGAAAAAAAAAATGGCTGTATCGGCCTGTATGGGACTGTAA
TGCGCTTAGTGGTCTGACATATACTGGAAATGTATGTATACTGGCGTACT
TTATATTCTCTAAAATGCTTAATGCCTTTGAAATTTTGTAATCAAAAAAA
AGCTTTGAAAAAATCTAAAGGGGAGAGTATTCTTTAAAGTTTTTAACATA
AGCTTGTCAATGCACATGTAGATGGTTAGCATGTTTAGCAAACCTTGTGA
AATTATAATAAGTTTGTAGTTACATGTGAAACTCTAAATGCATGGCAACT
GTTAATGTCATAACAGTTTAGTTATTTTGTTCTGTTCTGTCATGTGCCAC
AAAATATGTACTTTTTTCACTTTTTTCCCTTTGTATATCAGTTACGGGTT
ACAACTGGTTCATTCTGAAAACAACAACAACAAAAGTCCATTCATATTTT
TTAACAATTGTATAAGTGCCCAAGTAATTCACTACAGCCTAAAGCCTTGC
CTTTGTAATTTGACTTCTGACATGTTGGCAATCAAAGCATGCACTTGTAA
CAATGAAAAAGAAAAAGCATTTTATATTACTACTCAATAAAATGTGCATG
AACTTACAGAATTCTCATCCTTCCACTGAGTCCGCTGAAGGGATTTATGT
GCACAACCACCATGTGTCTTCTAGGTGCTGGCCCACCACCACACATCACA
GGCTGATTTCCACAGGCTTCTTCCTAGGGGCCTCGTGATCTGAGGGGTGG
TGCCTACTTCCACTGTAAGAAAGAATCTTGGTGGATTTGTGTCTCAAATC
AGATAAGAGAAGCCTGTTTAAAGAGCAGATGCCATCTTCTGGCTTCCTCA
AGGAGCCAGTTAAAAAACCAGAGCATTCCTTTTTATTGAAAAATAAAATT
AATTTGTTATCAGGTTGTTTCAGTTGTATTGGATGCCCTATCTATCTGCT
AAAGCAAAAAGTACTAGGCTACTAAGTGCATTTTCATCACAGAAAAGAGT
TGCATTTGTATTAACAAGAAATTTGTATACCCACGCTTCAGCTACTATCT
AATCATCACCCGAAGATTTAAGATACACCAAATTTCAGTTTGTTTGTAAC
ATTGTTCATCTTTAGTGCACTTTGTTTTATATAATAAAGTATGCCTGTTA
TATTAAATAATAAGAATATGGCAATTAGCGATATGGCATACCCAAACAAA
GATGTTCTCGATACAGTCTGGCAAAGACTATCCCAAGGTTATTTTAATGA
ATTCAGACATTTTTTCCTGTGGATATTTCTCCATCCTAAAAAAAGTGGCA
ACCAAGGAAAATATTTAGATGCAACTTACTAGAGTGATGATGTGAAAGAA
ATGGTGATTCTGGTATCATGGTGTTTATTTTCTTTCTTATAACTGCAGAG
AAAATATCCTGACTAAAAAAATTTCATTTTTTTGGATTCCTTTCTTTTAC
AAATTGTGCTGAGGCAACTATGGCATAGAAATAAACATTTGACATTAAAA
TAAGTAAAAAAAAAAAAAAAAAAAA

The derived exemplary PCR primers for the amplification of the PPP3CA tDNA are summarized in the following Table 9.

TABLE 9
Examples for PPP3CA primer.

| SEQ | | | Tm | GC content |
|---|---|---|---|---|
| ID No. | Primer name | Primer sequence | [° C.] | [%] |
| 41 | PPP3CAs1 | AAGATGCAGGGTACCGCATG | 60.8 | 55.0 |
| 42 | PPP3CAas3 | ACTTCAATACTGCAGCTTTGTTATTG | 59.8 | 34.6 |
| 43 | PPP3CAas1 | ACTTCAATACTGCAGCTTTGTTATTG | 59.8 | 34.6 |
| 44 | PPP3CAs2 | CAATAACAAAGCTGCAGTATTGAAGT | 59.8 | 34.6 |
| 45 | PPP3CAas2 | GCTGTTGCACCATCAAATCCA | 61.6 | 47.6 |
| 46 | PPP3CAs3 | TGGATTTGATGGTGCAACAGC | 61.6 | 47.6 |

,,s“ means sense primer, ,,as“ means antisense primer; PPP3CA means protein phosphatase-3 (formerly 2B), catalytic subunit, alpha isoform (calcineurin A alpha) (PPP3CA).

### 3.8.1 Pre-PCR

The PCR amplificate PPP3CA-1, which is obtained by the pre-PCR by the use of the primers PPP3CAs1 (sense primer) and PPP3CAas3 (antisense primer), each of which at a concentration of 0.3 μM, has a length of 410 bp.

3.8.2 The PCR Amplificate PPP3CA-2, which is Obtained by the Gene-Specific Secondary PCR by the Use of the Primers PPP3CAs1 (Sense Primer) and PPP3CAaS1 (Antisense Primer), Each of which at a Concentration of 0.3 μM, has a Length of 120 bp.

The PCR amplificate PPP3CA-2, which is obtained by the gene-specific secondary PCR by the use of the primers PPP3CAs2 (sense primer) and PPP3CAas2 (antisense primer), each of which at a concentration of 0.3 μM, has a length of 222 bp.

The PCR amplificate PPP3CA-3 which is obtained by the gene-specific secondary PCR by the use of the primers PPP3CAs3 (sense primer) and PPP3CAas3 (antisense primer), each of which at a concentration of 0.3 μM, has a length of 115 bp.

3.9 Primers for the Detection of Gene Therapy or Doping with the tDNA Encoding the Vascular Endothelial Growth Factor

FIG. 10 shows the structure of the exons and introns of nine reference mRNA sequences for the known doping-relevant protein variants of vascular endothelial growth factor (VEGF). Only the exon intron transition between exon 1 to exon 5 is completely conserved and is used in this embodiment for the design of intron-spanning PCR primers.

The genomic DNA sequence of VEGF is shown in the region of the mRNA>chr7:99963074-99965972. The intron sequences, the coding sequence (cds) for the gene therapy- or doping relevant protein are black in color, the sense primer is bold and the antisense primer is underlined and not bold; sequences which can be used as sense as well as antisense primers are bold and underlined.¹ stands for the beginning or the end of primer 1,² stands for the beginning or the end of primer 2.

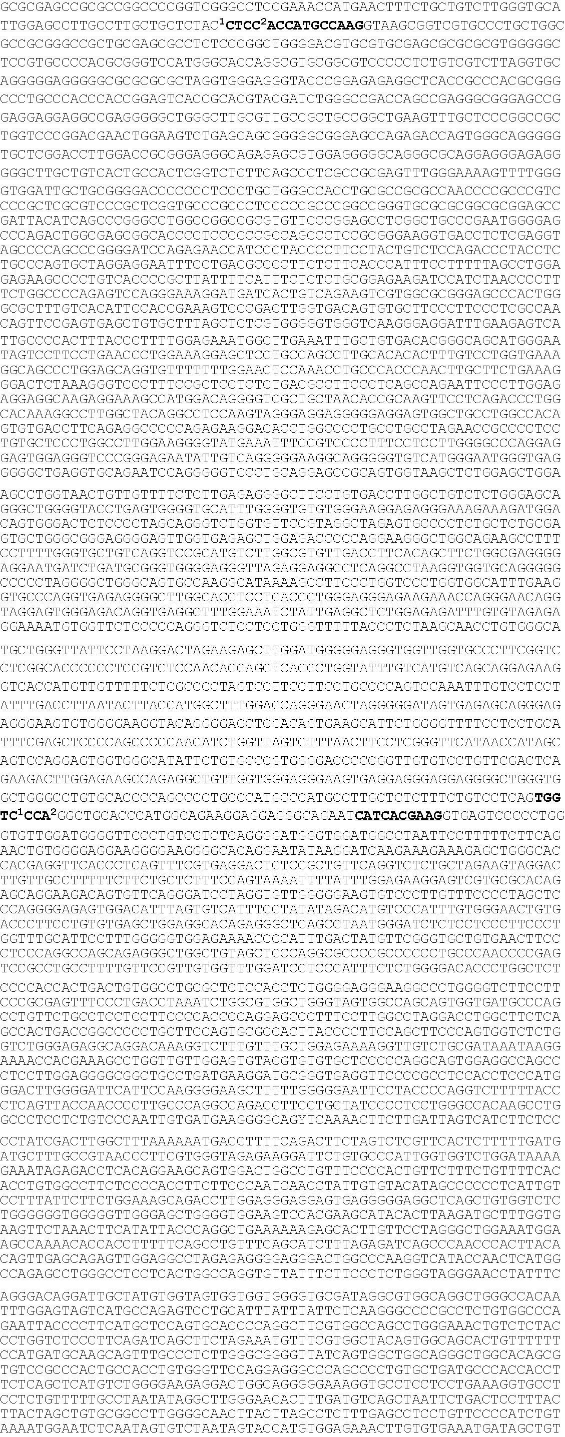

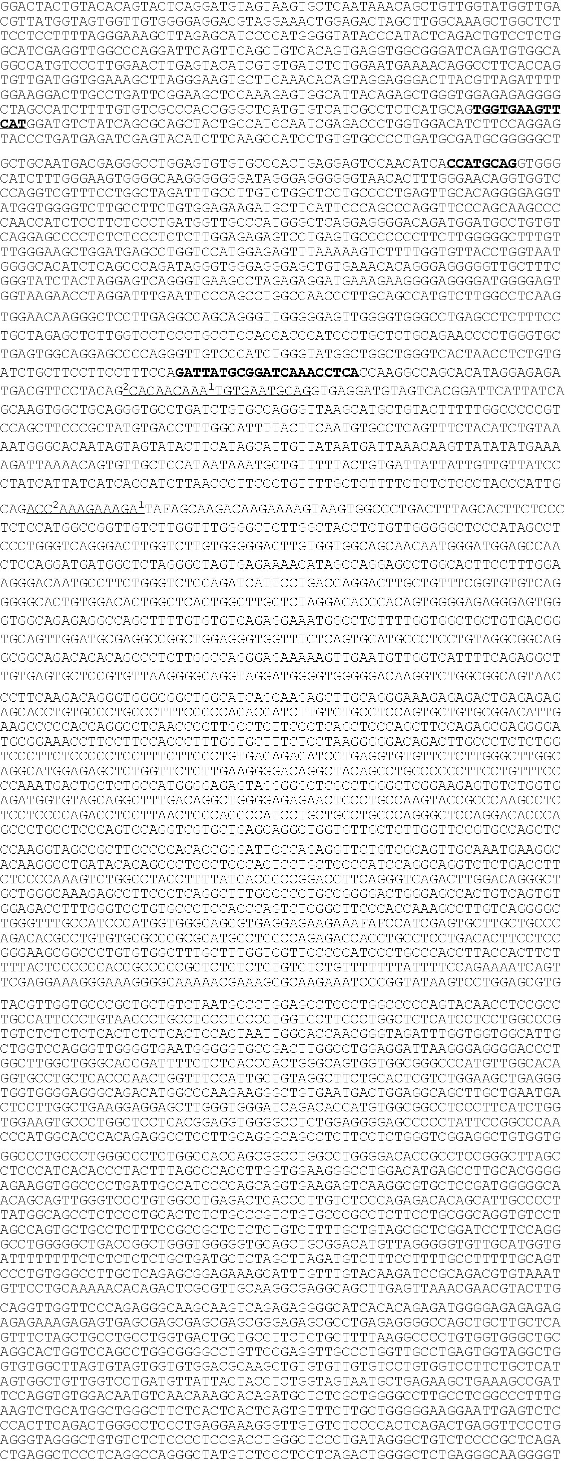

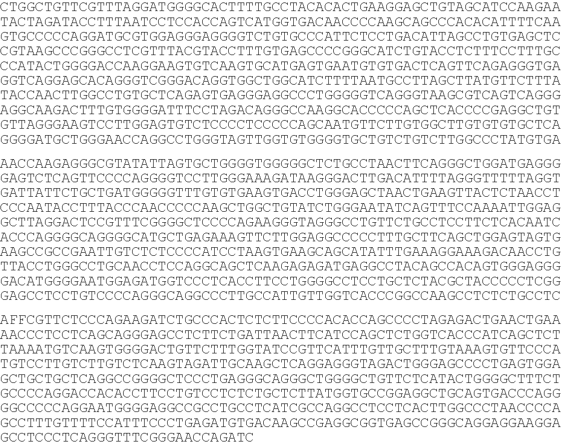

In the following the mRNA encoding human VEGF is shown:

Homo sapiens vascular endothelial growth factor
(VEGF) canonical mRNA region for all reference
mRNAs (see table 1) of exon 1 to exon 5:
GGCTTGGGGCAGCCGGGTAGCTCGGAGGTCGTGGCGCTGGGGGCTAGCAC
CAGCGCTCTGTCGGGAGGCGCAGCGGTTAGGTGGACCGGTCAGCGGACTC
ACCGGCCAGGGCGCTCGGTGCTGGAATTTGATATTCATTGATCCGGGTTT
TATCCCTCTTCTTTTTTCTTAAACATTTTTTTTTAAAACTGTATTGTTTC
TCGTTTTAATTTATTTTTGCTTGCCATTCCCCACTTGAATCGGGCCGACG
GCTTGGGGAGATTGCTCTACTTCCCCAAATCACTGTGGATTTTGGAAACC
AGCAGAAAGAGGAAAGAGGTAGCAAGAGCTCCAGAGAGAAGTCGAGGAAG
AGAGAGACGGGGTCAGAGAGAGCGCGCGGGCGTGCGAGCAGCGAAAGCGA
CAGGGGCAAAGTGAGTGACCTGCTTTTGGGGGTGACCGCCGGAGCGCGGC
GTGAGCCCTCCCCCTTGGGATCCCGCAGCTGACCAGTCGCGCTGACGGAC
AGACAGACAGACACCGCCCCCAGCCCCAGCTACCACCTCCTCCCCGGCCG
GCGGCGGACAGTGGACGCGGCGGCGAGCCGCGGGCAGGGGCCGGAGCCCG
CGCCCGGAGGCGGGGTGGAGGGGGTCGGGGCTCGCGGCGTCGCACTGAAA
CTTTTCGTCCAACTTCTGGGCTGTTCTCGCTTCGGAGGAGCCGTGGTCCG
CGCGGGGGAAGCCGAGCCGAGCGGAGCCGCGAGAAGTGCTAGCTCGGGCC
GGGAGGAGCCGCAGCCGGAGGAGGGGGAGGAGGAAGAAGAGAAGGAAGAG
GAGAGGGGGCCGCAGTGGCGACTCGGCGCTCGGAAGCCGGGCTCATGGAC
GGGTGAGGCGGCGGTGTGCGCAGACAGTGCTCCAGCCGCGCGCGCTCGAA
ACCATGAACTTTCTGCTGTCTTGGGTGCATTGGAGCCTTGCCTTGCTGCT
CTAC¹CTCC²ACCATGCCAAGTGGTC¹CCA²GGCTGCACCCATGGCAGAA
GGAGGAGGGCAGAATCATCACGAAGTGGTGAAGTTCATGGATGTCTATCA
GCGCAGCTACTGCCATCCAATCGAGACCCTGGTGGACATCTTCCAGGAGT
ACCCTGATGAGATCGAGTACATCTTCAAGCCATCCTGTGTGCCCCTGATG
CGATGCGGGGGCTGCTGCAATGACGAGGGCCTGGAGTGTGTGCCCACTGA
GGAGTCCAACATCACCATGCAGATTATGCGGATCAAACCTCACCAAGGCC
AGCACATAGGAGAGATGAGCTTCCTACAG²CACAACAAA¹TGTGAATGCA
GACC²AAAGAAAGA¹TAGAGCAAGACAAG

The derived exemplary PCR primers for the amplification of VEGF tDNA are summarized in the following Table 10.

TABLE 10
Example for VEGF-primer.

| SEQ | | | Tm | GC content |
|---|---|---|---|---|
| ID No. | Primer name | Primer sequence | [° C.] | [%] |
| 47 | VEGFs1 | CTCCACCATGCCAAGTGGTC | 60.7 | 60.0 |
| 48 | VEGFas3 | TCTTTCTTTGGTCTGCATTCACA | 60.1 | 39.1 |
| 49 | VEGFs1-II | ACCATGCCAAGTGGTCCCA | 61.3 | 57.9 |
| 50 | VEGFas1 | ATGAACTTCACCACTTCGTGATG | 59.2 | 43.5 |
| 51 | VEGFs2 | CATCACGAAGTGGTGAAGTTCAT | 59.2 | 43.5 |
| 52 | VEGFas2 | ATGAACTTCACCACTTCGTGATG | 62.1 | 66.7 |
| 53 | VEGFs3 | CCATGCAGATTATGCGGATCA | 61.7 | 47.6 |
| 54 | VEGFas3-II | GGTCTGCATTCACATTTGTTGTG | 60.5 | 43.5 |

,,s“ means sense primer, ,,as“ means antisense primer; VEGF means vascular entothelial growth factor.

### 3.9.1 Pre-PCR

The PCR amplificate VEGF1-3, which is obtained by the pre-PCR by the use of the primers VEGFs1 (sense primer) and VEGFas3 (antisense primer), each of which at a concentration of 0.3 μM, has a length of 353 bp.

### 3.9.2 Secondary PCR

The PCR amplificate of VEGF1, which is obtained by the gene-specific secondary PCR by the use of the primers VEGFs 1-II (sense primer) and VEGFas1 (antisense primer), each of which at a concentration of 0.3 μM, has a length of 80 bp.

The PCR amplificate of VEGF2, which is obtained by the gene-specific secondary PCR by the use of the primers VEGFs2 (sense primer) and VEGFas2 (antisense primer), each of which at a concentration of 0.3 μM, has a length of 220 bp.

The PCR amplificate VEGF3, which is obtained by the gene-specific secondary PCR by the use of the primers VEGFs3 (sense primer) and VEGFas3 (antisense primer), each of which at a concentration of 0.3 μM, has a length of 97 bp.

The PCR amplificate VEGF1-3-II, which is obtained by the gene-specific secondary PCR by the use of the primer pair VEGFs1-II (sense primer) and VEGFas3-2 (antisense primer) has a length of 340 bp.

### Example 5

### Assembly of Gene Therapy or Gene Doping Test Kits

With the PCR primers which were obtained in the example 4 an gene therapy or gene doping test kit is provided. Each test kit can be used for the detection of one specific tDNA. Consequently they differ in the specific primer pairs for the tDNA to be detected, contained in the solutions 1 to 3 in the corresponding products 1 to 3, the controls 1 to 3, and the sequences of the sense primers 1 to 3.

In the following by the way of example the assembly of gene therapy or gene doping test kits for 50 tests (A- and B-sample) for gene doping by means of EPO rDNA is shown:

### Solution 1 (350 μl):

### EPOs1: 3.0 μM

### EPOas3: 3.0 μM

In 350 μl pure water PCR-grade

### Solution 2 (350 μl):

### EPOs1-II: 3.0 μM

### EPOas1: 3.0 μM

In 350 μl pure water PCR-grade

### Solution 3 (350 μl):

### EPOs2+3: 3.0 μM

### EPOas2: 3.0 μM

In 350 μl pure water PCR-grade

### Solution 4 (350 μl):

### EPOs2+3: 3.0 μM

### EPOas3II: 3.0 μM

In 350 μl pure water PCR-grade

### Solution 5 (350 μl):

### EPOs1-II: 3.0 μM

### EPOas3-II: 3.0 μM

In 350 μl pure water PCR-grade

### Solution 6 (2 ml):

PCR product EPO1-3 cDNA 0.4 fg (femtogramm) in 1 ml pure water PCR-grade corresponding to one copy of EPO1-3 cDNA in one μl.

### Solution 7 (3 ml):

Human total DNA from whole blood 6 mg in 3 ml, corresponding to about 5.6×10⁵ molecules DNA in one μl.

### Solution 8 (5.6 ml):

70 U HotStarTaq™ DNA polymerase (Qiagen, Hilden, Germany) 28 μl
10× HotStarTaq™ DNA polymerase 10×PCR buffer 560 μl
ATP, TTP, GTP, CTP PCR-grade (Preqlab, Germany) each at 10 mM 112 μl
25 mM magnesium chloride 112 μl
pure water PCR-grade 4788 μl

### Solution 9 (100 μl):

10 μM EPOs1-II in 100 μl pure water PCR-grade

### Solution 10 (100 μl):

10 μM EPOs2+3 in 100 μl pure water PCR-grade

### Solution 11 (100 μl):

10 μM EPOs3-II in 100 μl pure water PCR-grade

### Solution 12 (20 ml):

pure water PCR-grade 20 ml

### Example 6

### Performance of the Method

### 6.1 Withdrawal of Samples

A sufficient amount of non-bioptic material is withdrawn from a person to be tested, which contains a concentration of about 50 μg of total DNA. This corresponds e.g. to 8 to 10 ml of whole blood which is withdrawn by the puncture of a peripheral vein. Such a measure corresponds to the guidelines of the WADA (World Antidoping Agency) for the performance of doping tests. With other non-bioptic samples, such as urine, a concentration of the sample by means of methods well-known in the art might be necessary.

6.2 Isolation of DNA (for Example from Whole Blood)

DNA is isolated from 8-10 ml whole blood that has been duly stored and handled, and 150 μl pure water PCR-grade was added. With a proper isolation the obtained DNA from a blood sample (DNABS) comprises a concentration of about 1.4 to 3.0 μg/μl corresponding to about 4.0 to 8.0×10⁵/μl molecules DNA (copies gDNA) and is sufficient for the performance of A- and B-sample tests with four different gene doping test kits.

### 6.3 PCR Reactions

PCRs using the different test kits and the DNABS sample as well as the controls 1 to 3 are performed as follows:

### Pre-PCRs:

I) Test person sample:
In a PCR tube 5 μl water PCR-grade+15 μl DNABS+5 μl solution 1+25 μl test kit solution 8
   II) Negative control PCR:
In a PCR tube 5 μl water PCR-grade+15 μl solution+5 μl solution 1+25 μl test kit solution 8

### III) Positive Control PCR:

In a PCR tube 5 μl solution 6+15 μl solution 7+5 μl solution 1+25 μl test kit solution 8

### Secondary PCRs:

The following secondary PCRs are run for each pre-PCR I-III:

EPO1 for I-III: In each PCR tube 1 μl PCR product 1, II or III+5 μl solution 2+25 μl test kit solution 8 and 19 μl water PCR-grade
EPO2 for I-III: In each PCR tube 1 μl PCR product 1, II or III+5 μl solution 3+25 μl test kit solution 8 and 19 μl water PCR-grade
EPO3 for I-III: In each PCR tube 1 μl PCR product 1, II or III+5 μl solution 4+25 μl test kit solution 8 and 19 μl water PCR-grade
EPO1-3-II for I-III: In each PCR tube 1 μl PCR product 1, II or III+5 μl solution 5+25 μl test kit solution 8 and 19 μl water PCR-grade

All of the 15 PCRs (3 pre- and 12 secondary PCRs) are subjected to an appropriate thermocycler on the following conditions. An appropriate thermocycler for the following protocol has a temperature ramp rate of at least 2° C. per second.

PCR conditions for the example:

Pre-PCR and secondary PCR: Activation at 95° C. for 15 min, followed by 35 cycles of annealing at 25 sec each at 59° C., 30 sec extension at 72° C. and denaturation at 94° C. for 15 sec.

### 6.4 Gel Electrophoresis

The 12 post-PCR products are subjected to separation by gel electrophoresis followed by a DNA staining according to standard protocols, wherein up to 25 μl of each sample are used. The test (A-sample) is referred positive if in one of the four secondary PCRs from I of the pre-PCR results in a band which position corresponds to its corresponding positive control band from II of the pre-PCR and to the known position due to the known mass for EPO1, EP2, EPO3 or EPO1-3-II. Simultaneously all negative controls (secondary PCRs from III) have to be negative.

### 6.5 B-Sample and Sequencing

If required a B-sample can be established by a repetition of the pre- and secondary PCRs. If the appearance of the band(s) from the patient sample can be reproduced the remaining volumes of the positive PCRs can be added to the solutions 9-11 as follows and subsequently sequenized:

EPO1 to solution 9 (9 μl EPO1+1 μl solution 9)

EPO2 to solution 10 (9 μl EPO1+1 μl solution 10)

EPO3 to solution 11 (9 μl EPO1+1 μl solution 11)

EPO1-3-II to solution 9 (9 μl EPO1+1 μl solution 9)

6.6 Embodiment for the Detection of EPO tDNA

In the following a negative and positive control assay is described which can be performed with the test kit for the detection of gene doping by means of EPO tDNA.

### 6.6.1 PCR Reactions:

### Pre-PCRs:

Positive control PCRs in 5 dilution stages (cDNA/gDNA):

I) In one PCR tube 2 μl solution 6+18 μl solution 7+5 μl solution 1+25 μl test kit solution 8 Corresponding to 2 copies of cDNA in relation to 10 million copies of gDNA
II) In one PCR tube 2 μl solution 6+3.6 μl solution 7+14.4 μl water PCR-grade+5 μl solution 1+25 μl test kit solution 8

Corresponding to 2 copies of cDNA in relation to 2 million copies of gDNA

III) In one PCR tube 4 μl solution 6+1.8 μl solution 7+14.2 μl water PCR-grade+5 μl solution 1+25 μl test kit solution 8

Corresponding to 4 copies of cDNA in relation to 1 million copies of gDNA

VI) In one PCR tube 10 μl solution 6+0.9 μl solution 7+9.1 μl water PCR-grade+5 μl solution 1+25 μl test kit solution 8

Corresponding to 10 copies of cDNA in relation to 500,000 copies of gDNA

Negative control PCR:

V) In one PCR tube 2 μl water PCR-grade+18 μl solution 7+5 μl solution 1+25 μl test kit solution 8 Corresponding to 0 copies of cDNA in relation to 10 million copies of gDNA
Expected PCR products I-V: EPO1-3 (437 bp)

### Secondary PCRs

The following secondary PCRs are in each case run for the pre-PCRs I-V:

1 for I-V: In each PCR tube 1 μl PCR product 1, II or III+5 μl solution 2+25 μl test kit solution 8 and 19 μl water PCR-grade

Expected PCR products I1-VI: EPO1 (169 bp)

2 for I-V: In each PCR tube 1 μl PCR product 1, II or III+5 μl solution 3+25 μl test kit solution 8 and 19 μl water PCR-grade

Expected PCR products 12-V2: EPO2 (109 bp)

3 for I-V: In each PCR tube 1 μl PCR product 1, II or III+5 μl solution 4+25 μl test kit solution 8 and 19 μl water PCR-grade

Expected PCR products 13-V3: EPO3 (289 bp)

4 for I-V: In each PCR tube 1 μl PCR product 1, II or III+5 μl solution 5+25 μl test kit solution 8 and 19 μl water PCR-grade

Expected PCR products 14-V4: EPO1-3III (423 bp)

All of the 15 PCRs (3 pre- and 12 secondary PCRs) are performed in an appropriate thermocycler on the following conditions. An appropriate thermocycler for the following protocol has a temperature ramp rate of at least 2° C. per second.

Conditions:

Pre-PCR and secondary PCR: Activation at 95° C. for 15 min, followed by 35 cycles of annealing of 25 sec at 95° C. each, 30 sec extension at 72° C. and denaturation at 94° C. for 15 sec.

### 6.6.2 Gel Electrophoresis

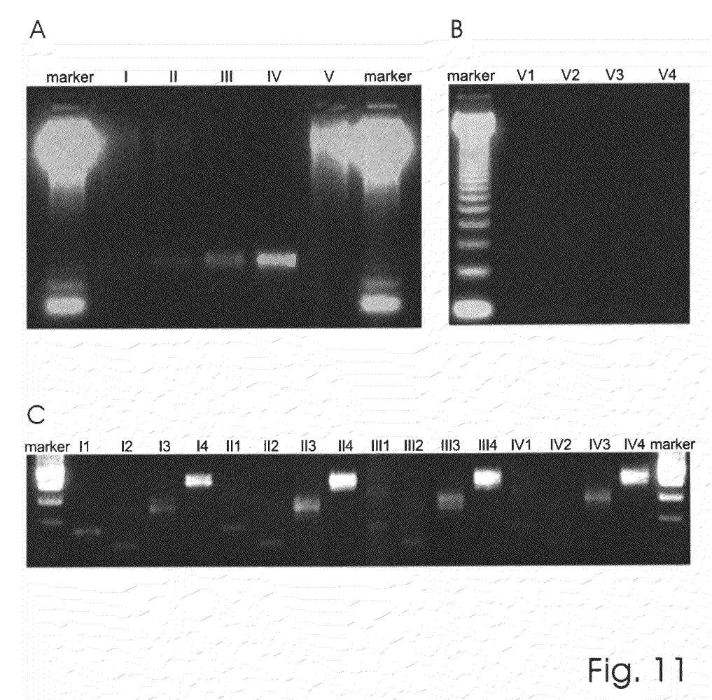

The result of the gel electrophoretical separation of the PCR products of the Pre-PCR (A) and the corresponding secondary PCR products (B and C) on an 1.5% agarose gel is shown in FIG. 11.

Pre- and secondary PCRs were performed according to the protocol for the test kit prototype as described. On the gel A an increasing yield of the PCR product “EPO1-3” with the expected size of 437 bp of I-V can be seen. The negative control (V) was negative for all 4 secondary PCR products (B).

In C the secondary PCR products I1-IV1 corresponding to “EPO1” (169 bp), 12-IV2 corresponding to “EPO2” (109 bp), 13-IV3 corresponding to “EPO3” (289 bp) as well as 14-IV4 corresponding to “EPO1-3III” (423 bp) are shown. It becomes obvious that especially for a safe detection of the PCR product and any subsequent sequencing a single pre-PCR which in this example encompasses 35 cycles, is not sufficient for a detection if the gDNA is present in highly diluted form (in A, lines I-III). After a subsequent secondary PCR at the given protocol conditions, the large PCR products EPO1-3II in I4-IV4 can be well evaluated and exist in sufficient amounts for a sequencing (>100 ng dsDNA).

We claim:

1. Method for the detection of transgenic DNA (tDNA) in a living being, which comprises the following steps:
(1) provision of a biological sample originating from said living being,
(2) analysis of said biological sample for the presence of tDNA, and
(3) correlation of a positive finding in step (2) with a positive detection of tDNA in said living being,
wherein said biological sample is a non-bioptic sample.

2. Method according to claim 1, wherein said non-bioptic sample is a blood sample.

3. Method according to claim 1, wherein step (2) comprises the following steps:
2.1 isolation of genetic material contained in the biological sample, and
2.2 performing a polymerase chain reaction (PCR) on said isolated genetic material,
and wherein step (3) comprises the following step:
3.1 correlation of the obtainment of an amplificate in step (2.2) with a positive detection of tDNA in said living being.

4. Method according to claim 3, wherein in step (2.2) such a PCR primer pair is used wherein the first PCR primer can hybridize at stringent conditions to a first exon on the first strand of the tDNA, and the second PCR primer can hybridize at stringent conditions to a second exon on the strand of the tDNA complementary to said first strand, which second exon is located downstream to said first exon.

5. Method according to claim 3, wherein in step (2.2) at least one of said two PCR primers is designed in such a manner that it can hybridize with a first segment to a first exon and simultaneously with a second segment to a second exon of said tDNA (intron-spanning PCR primer).

6. Method according to claim 5, wherein said at least one intron-spanning PCR primer is designed in such a manner that it can hybridize to such regions of said first and said second exons on said tDNA, which are conserved among splice variants of such genes from which the coding sequence of the tDNA derives.

7. Method according to claim 3, wherein in step (2.2) a nested PCR is performed which comprises a pre-PCR and a subsequent secondary PCR.

8. Method according to claim 1, wherein such a tDNA is to be detected which encodes doping-relevant proteins or such proteins which are of relevance for a somatic gene therapy.

9. Method according to claim 8, wherein said proteins are selected from the group consisting of: erythropoietin (EPO), growth hormone 1 (GH1), growth hormone 2 (GH2), insulin-like growth factor-1 (IGF1), insulin-like growth factor-2 (IGF2), myogenin, peroxisome proliferator-activated receptor delta (PPARd), calcineurin-A-alpha, vascular endothelial growth factor (VEGF), chorionic somatomammo-tropin hormone 1 (CSH1), chorionic somatomammo-tropin hormone 1/2 (CSH1/CSH2), chorionic somatomammo-tropin hormone 2 (CSH2), chorionic somatomammo-tropin hormone-like 1 (CSHL1), and myostatin inhibitor.

10. Kit which comprises a manual for performing the method according to claim 1 and reagents required for performing the method.


SEQUENCE LISTING (54 sequences)

SEQ ID NO: 1 - DNA, 19 bp, Artificial sequence
  Synthetic oligonucleotide
caatggctacaggctcccg

SEQ ID NO: 2 - DNA, 18 bp, Artificial sequence
  Synthetic oligonucleotide
gcaatggctgcaggctcc

SEQ ID NO: 3 - DNA, 18 bp, Artificial sequence
  Synthetic oligonucleotide
gcaatggctccaggctcc

SEQ ID NO: 4 - DNA, 23 bp, Artificial sequence
  Synthetic oligonucleotide
agcagctctaggttggatttctg

SEQ ID NO: 5 - DNA, 25 bp, Artificial sequence
  Synthetic oligonucleotide
gagcagctctaggttagatttctgc

SEQ ID NO: 6 - DNA, 24 bp, Artificial sequence
  Synthetic oligonucleotide
gagcagctctaagttggatttctg

SEQ ID NO: 7 - DNA, 24 bp, Artificial sequence
  Synthetic oligonucleotide
gagcagctctagattggatttctg

SEQ ID NO: 8 - DNA, 23 bp, Artificial sequence
  Synthetic oligonucleotide
cttccagcctaaactcctggtag

SEQ ID NO: 9 - DNA, 19 bp, Artificial sequence
  Synthetic oligonucleotide
atgggggtgcacgaatgtc

SEQ ID NO: 10 - DNA, 19 bp, Artificial sequence
  Synthetic oligonucleotide
atggcttccttctgggctc

SEQ ID NO: 11 - DNA, 19 bp, Artificial sequence
  Synthetic oligonucleotide
ggtgcacgaatgtcctgcc

SEQ ID NO: 12 - DNA, 21 bp, Artificial sequence
  Synthetic oligonucleotide
cacagcccgtcgtgatattct

SEQ ID NO: 13 - DNA, 21 bp, Artificial sequence
  Synthetic oligonucleotide
agaatatcacgacgggctgtg

SEQ ID NO: 14 - DNA, 18 bp, Artificial sequence
  Synthetic oligonucleotide
tgctgcccgacctccatc

SEQ ID NO: 15 - DNA, 21 bp, Artificial sequence
  Synthetic oligonucleotide
agaatatcacgacgggctgtg

SEQ ID NO: 16 - DNA, 19 bp, Artificial sequence
  Synthetic oligonucleotide
tccttctgggctcccagag

SEQ ID NO: 17 - DNA, 19 bp, Artificial sequence
  Synthetic oligonucleotide
ggtgcacgaatgtcctgcc

SEQ ID NO: 18 - DNA, 19 bp, Artificial sequence
  Synthetic oligonucleotide
tccttctgggctcccagag

SEQ ID NO: 19 - DNA, 24 bp, Artificial sequence
  Synthetic oligonucleotide
ccatgcctacagagtctgattttc

SEQ ID NO: 20 - DNA, 21 bp, Artificial sequence
  Synthetic oligonucleotide
aaacggattcagcccatcttc

SEQ ID NO: 21 - DNA, 26 bp, Artificial sequence
  Synthetic oligonucleotide
gcctacagagtctgattttctaatgc

SEQ ID NO: 22 - DNA, 22 bp, Artificial sequence
  Synthetic oligonucleotide
gattcagcccatcttctcctgg

SEQ ID NO: 23 - DNA, 24 bp, Artificial sequence
  Synthetic oligonucleotide
gtgatttcttgaaggtgaagatgc

SEQ ID NO: 24 - DNA, 23 bp, Artificial sequence
  Synthetic oligonucleotide
tgtgggcttgttgaaataaaagc

SEQ ID NO: 25 - DNA, 25 bp, Artificial sequence
  Synthetic oligonucleotide
atgattacacctacagtgaagatgc

SEQ ID NO: 26 - DNA, 22 bp, Artificial sequence
  Synthetic oligonucleotide
cttgaaggtgaagatgcacacc

SEQ ID NO: 27 - DNA, 24 bp, Artificial sequence
  Synthetic oligonucleotide
cttgttgaaataaaagcccctgtc

SEQ ID NO: 28 - DNA, 23 bp, Artificial sequence
  Synthetic oligonucleotide
cacctacagtgaagatgcacacc

SEQ ID NO: 29 - DNA, 24 bp, Artificial sequence
  Synthetic oligonucleotide
cttgttgaaataaaagcccctgtc

SEQ ID NO: 30 - DNA, 20 bp, Artificial sequence
  Synthetic oligonucleotide
gcggcttctacttcagcagg

SEQ ID NO: 31 - DNA, 19 bp, Artificial sequence
  Synthetic oligonucleotide
tggggaagttgtccggaag

SEQ ID NO: 32 - DNA, 19 bp, Artificial sequence
  Synthetic oligonucleotide
tacttcagcaggcccgcaa

SEQ ID NO: 33 - DNA, 19 bp, Artificial sequence
  Synthetic oligonucleotide
gttgtccggaagcacggtc

SEQ ID NO: 34 - DNA, 18 bp, Artificial sequence
  Synthetic oligonucleotide
caggggtgcccagcgaat

SEQ ID NO: 35 - DNA, 20 bp, Artificial sequence
  Synthetic oligonucleotide
gtgagcagatgatcccctgg

SEQ ID NO: 36 - DNA, 19 bp, Artificial sequence
  Synthetic oligonucleotide
gatgatcccctgggttggc

SEQ ID NO: 37 - DNA, 22 bp, Artificial sequence
  Synthetic oligonucleotide
cagcagctacacagacctctcc

SEQ ID NO: 38 - DNA, 21 bp, Artificial sequence
  Synthetic oligonucleotide
accaaaacggatagcgttgtg

SEQ ID NO: 39 - DNA, 21 bp, Artificial sequence
  Synthetic oligonucleotide
ctacacagacctctcccggag

SEQ ID NO: 40 - DNA, 21 bp, Artificial sequence
  Synthetic oligonucleotide
cggatagcgttgtgtgacatg

SEQ ID NO: 41 - DNA, 20 bp, Artificial sequence
  Synthetic oligonucleotide
aagatgcagggtaccgcatg

SEQ ID NO: 42 - DNA, 26 bp, Artificial sequence
  Synthetic oligonucleotide
acttcaatactgcagctttgttattg

SEQ ID NO: 43 - DNA, 26 bp, Artificial sequence
  Synthetic oligonucleotide
acttcaatactgcagctttgttattg

SEQ ID NO: 44 - DNA, 26 bp, Artificial sequence
  Synthetic oligonucleotide
caataacaaagctgcagtattgaagt

SEQ ID NO: 45 - DNA, 21 bp, Artificial sequence
  Synthetic oligonucleotide
gctgttgcaccatcaaatcca

SEQ ID NO: 46 - DNA, 21 bp, Artificial sequence
  Synthetic oligonucleotide
tggatttgatggtgcaacagc

SEQ ID NO: 47 - DNA, 20 bp, Artificial sequence
  Synthetic oligonucleotide
ctccaccatgccaagtggtc

SEQ ID NO: 48 - DNA, 23 bp, Artificial sequence
  Synthetic oligonucleotide
tctttctttggtctgcattcaca

SEQ ID NO: 49 - DNA, 19 bp, Artificial sequence
  Synthetic oligonucleotide
accatgccaagtggtccca

SEQ ID NO: 50 - DNA, 23 bp, Artificial sequence
  Synthetic oligonucleotide
atgaacttcaccacttcgtgatg

SEQ ID NO: 51 - DNA, 23 bp, Artificial sequence
  Synthetic oligonucleotide
catcacgaagtggtgaagttcat

SEQ ID NO: 52 - DNA, 23 bp, Artificial sequence
  Synthetic oligonucleotide
atgaacttcaccacttcgtgatg

SEQ ID NO: 53 - DNA, 21 bp, Artificial sequence
  Synthetic oligonucleotide
ccatgcagattatgcggatca

SEQ ID NO: 54 - DNA, 23 bp, Artificial sequence
  Synthetic oligonucleotide
ggtctgcattcacatttgttgtg